SEQUENCE LISTING (33 sequences)

SEQ ID NO: 1 - DNA, 734 bp, Homo sapiens
  gene: Unigene  Hs.7780; DCB  cDNA clone DCBATH02 5′ cDNA DKFZp564A072
aacagctcaaatcttcaaaatattactatagcattatgtttaaaataatctacaacaaaaatgtaccattttcaagcagtactacattaggagcccttttatagaaaataatttcttctttacccccgttccagtgtgaatctagtattctgttaacatttgtgtggcatttggagtttgtcatccccattgaagggagagccttctcagacatgaagcaagggaaacatactgaatagttttacacaaatttgatctggcttccatttgtccccctcatttcccaaatgtttaaatgtattggatttggattctcaatgtataagttgccttatctgttaatgtctatcttctgtctctttaattttgtatatctgctgttttgcttttggatacattttctaattagaagtcacatgataaatataatcagtatagtaataataccataatgtgcacatactcaataaataaatgactgcattgttgtaaatgaaaaaaaaaaaaaaaaanaaaaanaaaacccttgtcggccgcctcggcccagtcgactctagactcgagcaagcttatgcatgcggccgcaattcgagctcactgggccaattcgccctatagggagtcgtattacattccactgccgccgttttacaacgtcgtgactgggaaaaccctgccgttacccaacttaatgcgcttgaagcacattccctttcgccagctggcgtaaatgc

SEQ ID NO: 2 - DNA, 2279 bp, Homo sapiens
  gene: Unigene Hs.21293; Homo sapiens AgX-1 antigen mRNA, complete cds.
gaattcggggtggcgagaggggcggggtggccggggctgtctccacttggccccgctcccggcccgccccgccgccgccccccggatgagggtatatattcggagtgagcgcgggaccgatgagtggccgcgccgaaggagctggagacggtcgtagctgcggtcgccgagaaaggtttacaggtacatacattacacccctatttctacaaagcttggctattagagcattatgaacattaatgacctcaaactcacgttgtccaaagctgggcaagagcacctactacgtttctggaatgagcttgaagaagcccaacaggtagaactttatgcagagctccaggccatgaactttgaggagctgaacttctttttccaaaaggccattgaaggttttaaccagtcttctcaccaaaagaatgtggatgcacgaatggaacctgtgcctcgagaggtattaggcagtgctacaagggatcaagatcagctccaggcctgggaaagtgaaggacttttccagatttctcagaataaagtagcagttcttcttctagctggtgggcaggggacaagactcggcgttgcatatcctaaggggatgtatgatgttggtttgccatcccgtaagacactttttcagattcaagcagagcgtatcctgaagctacagcaggttgctgaaaaatattatggcaacaaatgcattattccatggtatataatgaccagtggcagaacaatggaatctacaaaggagttcttcaccaagcacaagtactttggtttaaaaaaagagaatgtaatcttttttcagcaaggaatgctccccgccatgagttttgatgggaaaattattttggaagagaagaacaaagtttctatggctccagatgggaatggtggtctttatcgggcacttgcagcccagaatattgtggaggatatggagcaaagaggcatttggagcattcatgtctattgtgttgacaacatattagtaaaagtggcagacccacggttcattggattttgcattcagaaaggagcagactgtggagcaaaggtggtagagaaaacgaaccctacagaaccagttggagtggtttgccgagtggatggagtttaccaggtggtagaatatagtgagatttccctggcaacagctcaaaaacgaagctcagacggacgactgctgttcaatgcggggaacattgccaaccatttcttcactgtaccatttctgagagatgttgtcaatgtttatgaacctcagttgcagcaccatgtggctcaaaagaagattccttatgtggatacccaaggacagttaattaagccagacaaacccaatggaataaagatggaaaaatttgtctttgacatcttccagtttgcaaagaagtttgtggtatatgaagtattgcgagaagatgagttttccccactaaagaatgctgatagtcagaatgggaaagacaaccctactactgcaaggcatgctttgatgtcccttcatcattgctgggtcctcaatgcagggggccatttcatagatgaaaatagctctcgccttccagcaattccccgcttgaaggatgccaatgatgtaccaatccaatgtgaaatctctcctcttatctcctatgctggagaaggattagaaagttatgtggcagataaagaattccatgcacctctaatcatcgatgagaatggagttcatgagctggtgaaaaatggtatttgaaccagataccaagttttgtttgccacgataggaatagcttttatttttgatagaccaactgtgaacctacaagacgtcttggacaactgaagtttaaatatccacagggttttattttgcttgttgaactcttagagctattgcaaacttcccaagatccagatgactgaatttcagatagcatttttatgattcccaactcattgaaggtcttatttatataattttttccaagccaaggagaccattggccatccaggaaatttcgtacagctgaaatataggcaggatgttcaacatcagtttacttgcagctggaagcatttgtttttgaagttgtacatagtaataatatgtcattgtacatgttgaaaggtttctatggtactaaaagtttgttttattttatcaaacattaagcttttttaagaaaataattgggcagtgaaataaatgtatcttcttgtctctggaaaaaaaaaaaaaaaaaaaaaaaaaaaaaaaaaaaaaaaaaaaaaaacccgaattc

SEQ ID NO: 3 - DNA, 2202 bp, Homo sapiens
  gene: Unigene Hs.79037; Homo sapiens heat shock 60kDa protein 1 (chaperonin) (HSPD1)
cacgcttgccgccgccccgcagaaatgcttcggttacccacagtctttcgccagatgagaccggtgtccagggtactggctcctcatctcactcgggcttatgccaaagatgtaaaatttggtgcagatgcccgagccttaatgcttcaaggtgtagaccttttagccgatgctgtggccgttacaatggggccaaagggaagaacagtgattattgagcagggttggggaagtcccaaagtaacaaaagatggtgtgactgttgcaaagtcaattgacttaaaagataaatacaagaacattggagctaaacttgttcaagatgttgccaataacacaaatgaagaagctggggatggcactaccactgctactgtactggcacgctctatagccaaggaaggcttcgagaagattagcaaaggtgctaatccagtggaaatcaggagaggtgtgatgttagctgttgatgctgtaattgctgaacttaaaaagcagtctaaacctgtgaccacccctgaagaaattgcacaggttgctacgatttctgcaaacggagacaaagaaattggcaatatcatctctgatgcaatgaaaaaagttggaagaaagggtgtcatcacagtaaaggatggaaaaacactgaatgatgaattagaaattattgaaggcatgaagtttgatcgaggctatatttctccatactttattaatacatcaaaaggtcagaaatgtgaattccaggatgcctatgttctgttgagtgaaaagaaaatttctagtatccagtccattgtacctgctcttgaaattgccaatgctcaccgtaagcctttggtcataatcgctgaagatgttgatggagaagctctaagtacactcgtcttgaataggctaaaggttggtcttcaggttgtggcagtcaaggctccagggtttggtgacaatagaaagaaccagcttaaagatatggctattgctactggtggtgcagtgtttggagaagagggattgaccctgaatcttgaagacgttcagcctcatgacttaggaaaagttggagaggtcattgtgaccaaagacgatgccatgctcttaaaaggaaaaggtgacaaggctcaaattgaaaaacgtattcaagaaatcattgagcagttagatgtcacaactagtgaatatgaaaaggaaaaactgaatgaacggcttgcaaaactttcagatggagtggctgtgctgaaggttggtgggacaagtgatgttgaagtgaatgaaaagaaagacagagttacagatgcccttaatgctacaagagctgctgttgaagaaggcattgttttgggagggggttgtgccctccttcgatgcattccagccttggactcattgactccagctaatgaagatcaaaaaattggtatagaaattattaaaagaacactcaaaattccagcaatgaccattgctaagaatgcaggtgttgaaggatctttgatagttgagaaaattatgcaaagttcctcagaagttggttatgatgctatggctggagattttgtgaatatggtggaaaaaggaatcattgacccaacaaaggttgtgagaactgctttattggatgctgctggtgtggcctctctgttaactacagcagaagttgtagtcacagaaattcctaaagaagagaaggaccctggaatgggtgcaatgggtggaatgggaggtggtatgggaggtggcatgttctaactcctagactagtgctttacctttattaatgaactgtgacaggaagcccaaggcagtgttcctcaccaataacttcagagaagtcagttggagaaaatgaagaaaaaggctggctgaaaatcactataaccatcagttactggtttcagttgacaaaatatataatggtttactgctgtcattgtccatgcctacagataatttattttgtatttttgaataaaaaacatttgtacattcctgatactgggtacaagagccatgtaccagtgtactgctttcaacttaaatcactgaggcatttttactactattctgttaaaatcaggattttagtgcttgccaccaccagatgagaagttaagcagcctttctgtggagagtgagaataattgtgtacaaagtagagaagtatccaattatgtgacaacctttgtgtaataaaaatttgtttaa

SEQ ID NO: 4 - DNA, 6914 bp, Homo sapiens
  gene: Unigene  Hs.30054; coagulation factor V (proaccelerin, labile factor)(F5)
tcattgcagctgggacagcccggagtgtggttagcagctcggcaagcgctgcccaggtcctggggtggtggcagccagcgggagcaggaaaggaagcatgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcagagaagatggacgacgctgtggctccaggccgagaatacacctatgaatggagtatcagtgaggacagtggacccacccatgatgaccctccatgcctcacacacatctattactcccatgaaaatctgatcgaggatttcaactctgggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcgaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaattaggtcattccgaaactcatcattgaaccaggaagaagaagagttcaatcttactgccctagctctggagaatggcactgaattcgtttcttcgaacacagatataattgttggttcaaattattcttccccaagtaatattagtaagttcactgtcaataaccttgcagaacctcagaaagccccttctcaccaacaagccaccacagctggttccccactgagacacctcattggcaagaactcagttctcaattcttccacagcagagcattccagcccatattctgaagaccctatagaggatcctctacagccagatgtcacagggatacgtctactttcacttggtgctggagaattcagaagtcaagaacatgctaagcgtaagggacccaaggtagaaagagatcaagcagcaaagcacaggttctcctggatgaaattactagcacataaagttgggagacacctaagccaagacactggttctccttccggaatgaggccctgggaggaccttcctagccaagacactggttctccttccagaatgaggccctgggaggaccctcctagtgatctgttactcttaaaacaaagtaactcatctaagattttggttgggagatggcatttggcttctgagaaaggtagctatgaaataatccaagatactgatgaagacacagctgttaacaattggctgatcagcccccagaatgcctcacgtgcttggggagaaagcacccctcttgccaacaagcctggaaagcagagtggccacccaaagtttcctagagttagacataaatctctacaagtaagacaggatggaggaaagagtagactgaagaaaagccagtttctcattaagacacgaaaaaagaaaaaagagaagcacacacaccatgctcctttatctccgaggacctttcaccctctaagaagtgaagcctacaacacattttcagaaagaagacttaagcattcgttggtgcttcataaatccaatgaaacatctcttcccacagacctcaatcagacattgccctctatggattttggctggatagcctcacttcctgaccataatcagaattcctcaaatgacactggtcaggcaagctgtcctccaggtctttatcagacagtgcccccagaggaacactatcaaacattccccattcaagaccctgatcaaatgcactctacttcagaccccagtcacagatcctcttctccagagctcagtgaaatgcttgagtatgaccgaagtcacaagtccttccccacagatataagtcaaatgtccccttcctcagaacatgaagtctggcagacagtcatctctccagacctcagccaggtgaccctctctccagaactcagccagacaaacctctctccagacctcagccacacgactctctctccagaactcattcagagaaacctttccccagccctcggtcagatgcccatttctccagacctcagccatacaaccctttctccagacctcagccatacaaccctttctttagacctcagccagacaaacctctctccagaactcagtcagacaaacctttctccagccctcggtcagatgcccctttctccagacctcagccatacaaccatttctctagacttcagccagacaaacctctctccagaactcagccatatgactctctctccagaactcagtcagacaaacctttccccagccctcggtcagatgcccatttctccagacctcagccatacaaccctttctctagacttcagccagacaaacctctctccagaactcagtcaaacaaacctttccccagccctcggtcagatgcccctttctccagaccccagccatacaaccctttctctagacctcagccagacaaacctctctccagaactcagtcagacaaacctttccccagacctcagtgagatgcccctctttgcagatctcagtcaaattccccttaccccagacctcgaccagatgacactttctccagaccttggtgagacagatctttccccaaactttggtcagatgtccctttccccagacctcagccaggtgactctctctccagacatcagtgacaccacccttctcccggatctcagccagatatcacctcctccagaccttgatcagatattctacccttctgaatctagtcagtcattgcttcttcaagaatttaatgagtcttttccttatccagaccttggtcagatgccatctccttcatctcctactctcaatgatacttttctatcaaaggaatttaatccactggttatagtgggcctcagtaaagatggtacagattacattgagatcattccaaaggaagaggtccagagcagtgaagatgactatgctgaaattgattatgtgccctatgatgacccctacaaaactgatgttaggacaaacatcaactcctccagagatcctgacaacattgcagcatggtacctccgcagcaacaatggaaacagaagaaattattacattgctgctgaagaaatatcctgggattattcagaatttgtacaaagggaaacagatattgaagactctgatgatattccagaagataccacatataagaaagtagtttttcgaaagtacctcgacagcacttttaccaaacgtgatcctcgaggggagtatgaagagcatctcggaattcttggtcctattatcagagctgaagtggatgatgttatccaagttcgttttaaaaatttagcatccagaccgtattctctacatgcccatggactttcctatgaaaaatcatcagagggaaagacttatgaagatgactctcctgaatggtttaaggaagataatgctgttcagccaaatagcagttatacctacgtatggcatgccactgagcgatcagggccagaaagtcctggctctgcctgtcgggcttgggcctactactcagctgtgaacccagaaaaagatattcactcaggcttgataggtcccctcctaatctgccaaaaaggaatactacataaggacagcaacatgcctgtggacatgagagaatttgtcttactatttatgacctttgatgaaaagaagagctggtactatgaaaagaagtcccgaagttcttggagactcacatcctcagaaatgaaaaaatcccatgagtttcacgccattaatgggatgatctacagcttgcctggcctgaaaatgtatgagcaagagtgggtgaggttacacctgctgaacataggcggctcccaagacattcacgtggttcactttcacggccagaccttgctggaaaatggcaataaacagcaccagttaggggtctggccccttctgcctggttcatttaaaactcttgaaatgaaggcatcaaaacctggctggtggctcctaaacacagaggttggagaaaaccagagagcagggatgcaaacgccatttcttatcatggacagagactgtaggatgccaatgggactaagcactggtatcatatctgattcacagatcaaggcttcagagtttctgggttactgggagcccagattagcaagattaaacaatggtggatcttataatgcttggagtgtagaaaaacttgcagcagaatttgcctctaaaccttggatccaggtggacatgcaaaaggaagtcataatcacagggatccagacccaaggtgccaaacactacctgaagtcctgctataccacagagttctatgtagcttacagttccaaccagatcaactggcagatcttcaaagggaacagcacaaggaatgtgatgtattttaatggcaattcagatgcctctacaataaaagagaatcagtttgacccacctattgtggctagatatattaggatctctccaactcgagcctataacagacctacccttcgattggaactgcaaggttgtgaggtaaatggatgttccacacccctgggtatggaaaatggaaagatagaaaacaagcaaatcacagcttcttcgtttaagaaatcttggtggggagattactgggaacccttccgtgcccgtctgaatgcccagggacgtgtgaatgcctggcaagccaaggcaaacaacaataagcagtggctagaaattgatctactcaagatcaagaagataacggcaattataacacagggctgcaagtctctgtcctctgaaatgtatgtaaagagctataccatccactacagtgagcagggagtggaatggaaaccatacaggctgaaatcctccatggtggacaagatttttgaaggaaatactaataccaaaggacatgtgaagaactttttcaaccccccaatcatttccaggtttatccgtgtcattcctaaaacatggaatcaaagtattgcacttcgcctggaactctttggctgtgatatttactagaattgaacattcaaaaacccctggaagagactctttaagacctcaaaccatttagaatgggcaatgtattttacgctgtgttaaatgttaacagttttccactatttctctttcttttctattagtgaataaaattttatac

SEQ ID NO: 5 - DNA, 1654 bp, Homo sapiens
  gene: Unigene Hs.75432; IMP (inosine monophosphate) dehydrogenase 2 (IMPDH2)
gaattcgggcggtcctcggagacacgcggcggtgtcctgtgttggccatggccgactacctgattagtgggggcacgtcctacgtgccagacgacggactcacagcacagcagctcttcaactgcggagacggcctcacctacaatgactttctcattctccctgggtacatcgacttcactgcagaccaggtggacctgacttctgctctgaccaagaaaatcactcttaagaccccactggtttcctctcccatggacacagtcacagaggctgggatggccatagcaatggcgcttacaggcggtattggcttcatccaccacaactgtacacctgaattccaggccaatgaagttcggaaagtgaagaaatatgaacagggattcatcacagaccctgtggtcctcagccccaaggatcgcgtgcgggatgtttttgaggccaaggcccggcatggtttctgcggtatcccaatcacagacacaggccggatggggagccgcttggtgggcatcatctcctccagggacattgattttctcaaagaggaggaacatgactgtttcttggaagagataatgacaaagagggaagacttggtggtagccccccgcagcatcacactgaaggaggcaaatgaaattctgcagcgcagcaagaagggaaagttgcccattgtaaatgaagatgatgagcttgtggccatcattgcccggacagacctgaagaagaatcgggactacccactagcctccaaagatgccaagaaacagctgctgtgtggggcagccattggcactcatgaggatgacaagtataggctggacttgctcgcccaggctggtgtggatgtagtggttttggactcttcccagggaaattccatcttccagatcaatatgatcaagtacatcaaagacaaataccctaatctccaagtcattggaggcaatgtggtcactgctgcccaggccaagaacctcattgatgcaggtgtggatgccctgcgggtgggcatgggaagtggctccatctgcattacgcaggaagtgctggcctgtgggcggccccaagcaacagcagtgtacaaggtgtcagagtatgcacggcgctttggtgttccggtcattgctgatggaggaatccaaaatgtgggtcatattgcgaaagccttggcccttggggcctccacagtcatgatgggctctctcctggctgccaccactgaggcccctggtgaatacttcttttccgatgggatccggctaaagaaatatcgcggtatgggttctctcgatgccatggacaagcacctcagcagccagaacagatatttcagtgaagctgacaaaatcaaagtggcccagggagtgtctggtgctgtgcaggacaaagggtcaatccacaaatttgtcccttacctgattgctggcatccaacactcatgccaggacattggtgccaagagcttgacccaagtccgagccatgatgtactctggggagcttaagtttgagaagagaacgtcctcagcccaggtggaaggtggcgtccatagcctccattcgtatgagaagcggcttttctgaaaagggatccagcacacctcctcggtttttttttcaataaaagtttagaaagacccgaattc

SEQ ID NO: 6 - DNA, 893 bp, Homo sapiens
  gene: Unigene Hs.699; peptidylprolyl isomerase B (cyclophilin B) (PPIB)
gggtttcgcctccgcctgtggatgctgcgcctctccgaacgcaacatgaaggtgctccttgccgccgccctcatcgcggggtccgtcttcttcctgctgctgccgggaccttctgcggccgatgagaagaagaaggggcccaaagtcaccgtcaaggtgtattttgacctacgaattggagatgaagatgtaggccgggtgatctttggtctcttcggaaagactgttccaaaaacagtggataattttgtggccttagctacaggagagaaaggatttggctacaaaaacagcaaattccatcgtgtaatcaaggacttcatgatccagggcggagacttcaccaggggagatggcacaggaggaaagagcatctacggtgagcgcttccccgatgagaacttcaaactgaagcactacgggcctggctgggtgagcatggccaacgcaggcaaagacaccaacggctcccagttcttcatcacgacagtcaagacagcctggctagatggcaagcatgtggtgtttggcaaagttctagagggcatggaggtggtgcggaaggtggagagcaccaagacagacagccgggataaacccctgaaggatgtgatcatcgcagactgcggcaagatcgaggtggagaagccctttgccatcgccaaggagtagggcacagggacatctttctttgagtgaccgtctgtgcaggccctgtagtccgccacagggctctgagctgcactggccccggtgctggcatctggtggagcggacccactcccctcacattccacaggcccatggactcacttttgtaacaaactcctaccaacactgaccaataaaaaaaaatgtgggttttttttttttttaatataaaaaaacccccc

SEQ ID NO: 7 - DNA, 1901 bp, Homo sapiens
  gene: Unigene Hs.1708; ; chaperonin containing TCP1, subunit 3 (gamma) (CCT3)
atggggcatcggccggtgctcgtgctcagccagaacacaaagcgtgaatccggaagaaaagttcaatctggaaacatcaatgctgccaagactattgcagatatcatccgaacatgtttgggacccaagtccatgatgaagatgcttttggacccaatgggaggcattgtgatgaccaatgatggcaatgccattcttcgagagattcaagtccagcatccagcggccaagtccatgatcgaaattagccggacccaggatgaagaagttggagatgggaccacatcagtaattattcttgcaggggaaatgctgtctgtagctgagcacttcctggagcagcagatgcacccaacagtggtgatcagtgcttaccgcaaggcattggatgatatgatcagcaccctaaagaaaataagtatcccagtcgacatcagtgacagtgatatgatgctgaacatcatcaacagctctattactaccaaagccatcagccggtggtcatctttggcttgcaacattgccctggatgctgtcaagatggtacagtttgaggagaatggtcggaaagagattgacataaaaaaatatgcaagagtggaaaagatacctggaggcatcattgaagactcctgtgtcttgcgtggagtcatgattaacaaggatgtgacccatccacgtatgcggcgctatatcaagaaccctcgcattgtgctgctggattcttctctggaatacaagaaaggaggaagccagactgacattgagattacacgagaggaggacttcacccgaattctccagatggaggaagagtacatccagcagctctgtgaggacattatccaactgaagcccgatgtggtcatcactgaaaagggcatctcagatttagctcagcactaccttatgcgggccaatatcacagccatccgcagagtccggaagacagacaataatcgcattgctagagcctgtggggcccggatagtcagccgaccagaggaactgagagaagatgatgttggaacaggagcaggcctgttggaaatcaagaaaattggagatgaatactttactttcatcactgactgcaaagaccccaaggcctgcaccattctcctccggggggctagcaaagagattctctcggaagtagaacgcaacctccaggatgccatgcaagtgtgtcgcaatgttctcctggaccctcagctggtgccagggggtggggcctccgagatggctgtcgcccatgccttgacagaaaaatccaaggccatgactggtgtggaacaatggccatacagggctgttgcccaggccctagaggtcattcctcgtaccctgatccagaactgtggggccagcaccatccgtctacttacctcccttcgggccaagcacacccaggagaactgtgagacctggggtgtaaatggtgagacgggtactttggtggacatgaaggaactgggcatatgggagccattggctgtgaagctgcagacttataagacagcagtggagacggcagttctgctactgcgaattgatgacatcgtttcaggccacaaaaagaaaggcgatgaccagagccggcaaggcggggctcctgatgctggccaggagtgagtgctaggcaaggctacttcaatgcacagaaccagcagagtctccccttttcctgagccagagtgccaggaacactgtggacgtctttgttcagaagggatcaggttggggggcagcccccagtccctttctgtcccagctcagttttccaaaagacactgacatgtaattcttctctattgtaaggtttccatttagtttgcttccgatgattaaatctaagtcatttgaaaaaaaaaaaaaaaaaaaaaaaaaaaaaaaaaaaa

SEQ ID NO: 8 - DNA, 1249 bp, Homo sapiens
  gene: Unigene Hs.69469; dendritic cell protein (GA17)
gggtcggcgtggtcttgcgagtggagtgtccgctgtgcccgggcctgcaccatgagcgtcccggccttcatcgacatcagtgaagaagatcaggctgctgagcttcgtgcttatctgaaatctaaaggagctgagatttcagaagagaactcggaaggtggacttcatgttgatttagctcaaattattgaagcctgtgatgtgtgtctgaaggaggatgataaagatgttgaaagtgtggtgaacagtgtggtatccctactcttgatcctggaaccagacaagcaagaagctttgattgaaagcctatgtgaaaagctggtcaaatttcgcgaaggtgaacgcccgtctctgagactgcagttgttaagcaaccttttccacgggatggataagaatactcctgtaagatacacagtgtattgcagccttattgaagtggtagcatcttgtggggccatccagtacatcccaactgagctggatcaagttagaaaatggatttctgactggaatctcaccactgaaaaaaagcacacccttttaagactactttatgaggcacttgcggattgtaagaagagtgatgctgcttcaaaagtcatggtggaattgctcggaagttacacagaggacaatgcttcccaggctcgagttgatgcccacaggtgtattgtcgaaccattgaaagatccaaatgcatttctttttgaccaccttcttactttaaaaccagtcaagtttttggaaggcgagcttattcatgatcttttaaccatttttgtgagtgctaaattggcatcatatgtcaagttttatcagaataataaagacttcattgattcacttggcctgttacatgaacagaatatggcaaaaatgagactacttacttttatgggaatggcaatagaaaataaggaaatttcttttgacacaatgcagcaagaacttcagattggagctgatgatgttgaagcatttgttattgacgccgtaagaactaaaatggtctactgcaaaattgatcagacccagagaaaagtagttgtcagtcatagcacacatcggacatttggaaaacagcggtggcaacaactgtatgacacacttaatgcctggaaacaaaatctgaacaaagtgaaaaacagccttttgagtctttccgatacctgagtttttatgcttataatttttgttctttgaaaaaaaagccctaaatcatagtaagacattataaaccaaaaaa

SEQ ID NO: 9 - DNA, 664 bp, Homo sapiens
  gene: Unigene Hs.82280; regulator of G-protein signaling 10 (RGS10)
ggattgttggtctgcgtggaacttctcaggtggacaccagagcatggaacacatccacgacagcgatggcagttccagcagcagccaccagagcctcaagagcacagccaaatgggcggcatccctggagaatctgctggaagacccagaaggcgtgaaaagatttagggaatttttaaaaaaggaattcagtgaagaaaatgttttgttttggctagcatgtgaagattttaagaaaatgcaagataagacgcagatgcaggaaaaggcaaaggagatctacatgacctttctgtccagcaaggcctcatcacaggtcaacgtggaggggcagtctcggctcaacgagaagatcctggaagaaccgcaccctctgatgttccagaaactccaggaccagatctttaatctcatgaagtacgacagctacagccgctttcttaagtctgacttgtttttaaaacacaagcgaaccgaggaagaggaagaagatttgcctgatgctcaaactgcagctaaaagagcttccagaatttataacacatgagcccccaaaaagccgggactggcagctttaagaagcaaaggaatttcctctcaggacgtgccgggtttatcattgctttgttatttgtaaggactgaaatgtacaaaacccttcaat

SEQ ID NO: 10 - DNA, 1792 bp, Homo sapiens
  gene: Unigene Hs.79217; pyrroline-5-carboxylate reductase 1 (PYCR1)
ctccggacagcatgagcgtgggcttcatcggcgctggccagctggcttttgccctggccaagggcttcacagcagcaggcgtcttggctgcccacaagataatggctagctccccagacatggacctggccacagtttctgctctcaggaagatgggggtgaagttgacaccccacaacaaggagacggtgcagcacagtgatgtgctcttcctggctgtgaagccacacatcatccccttcatcctggatgaaataggcgccgacattgaggacagacacattgtggtgtcctgcgcggccggcgtcaccatcagctccattgagaagaagctgtcagcgtttcggccagcccccagggtcatccgctgcatgaccaacactccagtcgtggtgcgggagggggccaccgtgtatgccacaggcacgcacgcccaggtggaggacgggaggctcatggagcagctgctgagcacggtgggcttctgcacggaggtggaagaggacctgattgatgccgtcacggggctcagtggcagcggccccgcctacgcattcacagccctggatgccctggctgatgggggtgtgaagatgggacttccaaggcgcctggcagtccgcctcggggcccaggccctcctgggggctgccaagatgctgctgcactcagaacagcacccaggccagctcaaggacaacgtcagctctcctggtggggccaccatccatgccttgcatgtgctggagagtgggggcttccgctccctgctcatcaacgctgtggaggcctcctgcatccgcacacgggagctgcagtccatggctgaccaggagcaggtgtcaccagccgccatcaagaagaccatcctggacaaggtgaagctggactcccctgcagggaccgctctgtcgccttctggccacaccaagctgctcccccgcagcctggccccagcgggcaaggattgacacgtcctgcctgaccaccatcctgccaccaccttctcttctcttgtcactagggggactagggggtccccaaagtggcccactttctgtggctctgatcagcgcaggggccagccagggacatagccagggaggggccacatcacttcccactggaaatctctgtggtctgcaagtgcttcccagcccagaacaggggtggattccccaacctcaacctcctttcttctctgctcccaaaccatgtcaggaccaccttcctctagagctcgggagcccggagggtcttcacccactcctactccagtatcagctggcacgggctccttcctgagagcaaaggtcaaggaccccctctgtgaaggctcagcagaggtgggatcccacgccccctcccggcccctccctgccctccattcagggagaaacctctccttcccgtgtgagaagggccagagggtccaggcatcccaagtccagcgtgaagggccacagcccctcttggctgccaagcacgcagatcccatggacatttggggaaagggctccttgggctgctggtgaacttctgtggccaccacctcctgctcctgacctccctgggagggtgctatcagttctgtcctggccctttcagttttataagttggtttccagcccccagtgtcctgacttctgtctgccacatgaggagggaggccctgcctgtgtgggagggtggttactgtgggtggaatagtggaggccttcaactgattagacaaggcccgcccacatcttggagggcatctgccttactgattaaaatgtcaatgtaatct

SEQ ID NO: 11 - DNA, 529 bp, Homo sapiens
  gene: Unigene Hs.117950; cDNA clone IMAGE1519390 3
aatcttctaagtccttttaattgttcttataaactagcataagatataaacttaagtagtacacatgagttttataatttactaatctctgacagatagctaagcatagcacatcagagcataacacagtgtgagggaaataaagtgtacaatgacatcttctattctggacctaataattcaatagagaaagaactacttgtagtcactgtggttacagaaggtttcatggacagcgaacataaagctctactagctaacaaataggtcttaatgataaaaacgtgggccttcagagaactaaaggtaccaatgtgtggcagtccaaaattacgaggaaaatgagttcccttcatgggtcacatcagcaattttttttttccccttttgagacagagtcttgctctgctgcccaggttggagtgcagtggcatgatccaggctcactgcaacctccgcctcccgggttcaagcaattctcatgcctcagcctcccgagtagctgggattacaggtgcctgtcatcacg

SEQ ID NO: 12 - DNA, 6031 bp, Homo sapiens
  gene: Unigene Hs.8858; bromodomain adjacent to zinc finger domain, 1A (BAZ1A)
cttttcccatcgtgtagtcaagagtctgtgccagacttgaaggctttactttgttagccatgtgtttatgaacccccagcgctttccctagatcttttggctgataatctcaaacatggaggatgcttctgaatcttcacgaggggttgctccattaattaataatgtagttctcccaggctctccgctgtctcttcctgtatcagtgacaggctgtaaaagtcatcgagtagccaataaaaaggtagaagcgaggagtgaaaagctcctcccaacagctcttcctccttcagagccgaaagtagatcagaaacttcccaggagctccgagaggcggggaagtggcggtgggacgcaattccccgcgcggagtcgggcagtggcagcgggagaagcggcagccaggggcgcggcggggccggagagaggcggtcccctgggaggacggggtctcccctcgttgcctttgtagtggagaaggtggacaagtggcagtcggcgtgatcgcagggaagcggggccggcgcgggcggccgagggtccaggcgagcccgcgggcggacgggagatgccgctgctacaccgaaagccgtttgtgagacagaagccgcccgcggacctgcggcccgacgaggaagttttctactgtaaagtcaccaacgagatcttccgccactacgatgacttttttgaacgaaccattctgtgcaacagccttgtgtggagttgtgctgtgacgggtagacctggactgacgtatcaggaagcacttgagtcagaaaaaaaagcaagacagaatcttcagagttttccagaaccactaattattccagttttatacttgaccagccttacccatcgttcgcgcttacatgaaatttgtgatgatatctttgcatatgtcaaggatcgatattttgtcgaagaaactgtggaagtcattaggaacaatggtgcaaggttgcagtgtaggattttggaagtcctccctccatcacatcaaaatggttttgctaatggacatgttaacagtgtggatggagaaactattatcatcagtgatagtgatgattcagaaacacaaagctgttcttttcaaaatgggaagaaaaaagatgcaattgatcccttactattcaagtataaagtgcaacccactaaaaaagaattacatgagtctgctattgttaaagcaacacaaatcagccggagaaaacacctattttctcgtgataaactaaagctttttctgaagcaacactgtgaaccacaagatggagtcattaaaataaaggcatcatctctttcaacgtataaaatagcagaacaagatttttcttatttcttccctgatgatccacccacatttatcttcagtcctgctaacagacgaagagggagacctcccaaacgaatacatattagtcaagaggacaatgttgctaataaacagactcttgcaagttataggagcaaagctactaaagaaagagataaacttttgaaacaagaagaaatgaagtcactggcttttgaaaaggctaaattaaaaagagaaaaagcagatgccctagaagcgaagaaaaaagaaaaagaagataaagagaaaaagagggaagaattgaaaaaaattgttgaagaagagagactaaagaaaaaagaagaaaaagagaggcttaaagtagaaagagaaaaggaaagagagaagttacgtgaagaaaagcgaaagtatgtggaatacttaaaacagtggagtaaacctagagaagatatggaatgtgatgaccttaaggaacttccagaaccaacaccagtgaaaactagactacctcctgaaatctttggtgatgctctgatggttttggagttccttaatgcatttggggaactttttgatcttcaagatgagtttcctgatggagtaaccctagaagtattagaggaagctcttgtaggaaatgacagtgaaggcccactgtgtgaattgctttttttcttcctgactgcaatcttccaggcaatagctgaagaagaagaggaagtagccaaagagcaactaactgatgctgacaccaaagatttaacagaggctttggatgaagatgcagaccccacaaaatctgcactgtctgcagttgcatctttggcagctgcatggccacagttacaccagggctgcagtttgaaaagtttggatcttgatagctgcactctttcagaaatcctcagactgcacatcttagcttcaggtgctgatgtaacatcagcaaatgcaaagtatagatatcaaaaacgaggaggatttgatgctacagatgatgcttgtatggagcttcgtttgagcaatcccagtctagtgaagaaactgtcaagcacctcagtgtatgatttgacaccaggagaaaaaatgaagatactccatgctctctgtggaaagctactgaccctagtttcaactagggattttattgaagattatgttgatatattacgacaggcaaagcaggagttccgggaattaaaagcagaacaacatcgaaaagagagggaagaagcagctgccagaattcgtaaaaggaaggaagaaaaacttaaggagcaagaacaaaaaatgaaagagaaacaagaaaaactgaaagaagatgagcaaagaaattcaacggcagatatatctattggggaggaagaaagggaagattttgatactagcattgagagcaaagacacagagcaaaaggaattagatcaagatatggtcactgaagatgaagatgacccaggatcacataaaagaggcagaagggggaaaagaggacaaaatggatttaaagaatttacaaggcaagaacagatcaactgtgtaacaagagagcctcttactgctgatgaggaagaagcattaaaacaggaacaccaacgaaaagagaaagagctcttagaaaaaatccaaagtgccatagcctgtaccaatatctttcccttgggtcgcgaccgcatgtatagacgatactggattttcccttctattcctggactctttattgaagaggattattctggtcttactgaagacatgctgttgcctagaccttcatcatttcagaataatgtacagtctcaagatcctcaggtatccactaaaactggagagcctttgatgtctgaatctacctccaacattgaccaaggtccacgtgaccattctgtgcagctgccaaaaccagtgcataagccaaatcggtggtgcttttacagttcttgtgaacagctagaccagcttattgaagctcttaattctagaggacatagagaaagtgccttaaaagaaactttgttacaagagaaaagcagaatatgtgcacagctagcccgtttttctgaagagaaatttcatttttcagacaaacctcagcctgatagcaaaccaacatatagtcggggaagatcttccaatgcatatgatccatctcagatgtgtgcagaaaagcaacttgaactaaggctgagagattttcttttagatattgaagatagaatctaccaaggaacattaggagccatcaaggttacagatcgacatatctggagatcagcattagaaagtggacggtatgagctgttaagtgaggaaaacaaggaaaatgggataattaaaactgtgaatgaagacgtagaagagatggaaattgatgaacaaacaaaggtcatagtaaaagacagacttttggggataaaaacagaaactccaagtactgtatcaacaaatgcaagtacaccacaatcagtgagcagtgtggttcattatctggcaatggcactctttcaaatagagcagggcattgagcggcgttttctgaaagctccacttgatgccagtgacagtgggcgttcttataaaacagttctggaccgttggagagagtctctcctttcttctgctagtctatcccaagtttttcttcacctatccaccttggatcgtagcgtgatatggtctaaatctatactgaatgcgcgttgcaagatatgtcgaaagaaaggcgatgctgaaaacatggttctttgtgatggctgtgataggggtcatcatacctactgtgttcgaccaaagctcaagactgtgcctgaaggagactggttttgtccagaatgtcgaccaaagcaacgttctagaagactctcctctagacagagaccatccttggaaagtgatgaagatgtggaagacagtatgggaggtgaggatgatgaagttgatggcgatgaagaagaaggtcaaagtgaggaggaagagtatgaggtagaacaagatgaagatgactctcaagaagaggaagaagtcagcctacccaaacgaggaagaccacaagttagattgccagttaaaacaagagggaaacttagctcttctttctcaagtcgtggccaacaacaagaacctggaagatacccttcaaggagtcagcagagcacacccaaaacaactgtttcttctaaaactggtagaagcctaagaaagataaactctgctcctcctacagaaacaaaatctttaagaattgccagtcgttctactcgccacagtcatggcccactgcaagcagatgtatttgtggaattgcttagtcctcgtagaaaacgcagaggcaggaaaagtgctaataatacaccagaaaatagtcccaacttccctaacttcagagtcattgccacaaagtcaagtgaacagtcaagatctgtaaatattgcttcaaaactttctctccaagagagtgaatccaaaagaagatgcagaaaaagacaatctccagagccatcgcctgtgacactgggtcgaaggagttctggccgacagggaggagttcatgaattgtctgcttttgaacaacttgttgtagaattggtacgacatgatgacagctggccttttttgaaacttgtttctaaaatccaggtcccagactactatgacatcatcaaaaagcccattgccttaaatataattcgtgaaaaagtgaataagtgtgaatataaattagcatctgagtttattgatgacattgagttaatgttttcgaactgctttgaatacaaccctcgtaacacaagtgaagcaaaagctggaactaggcttcaagcattttttcatattcaggctcaaaagcttggactccacgtcacacccagtaatgtggaccaagttagcacaccaccggctgcgaaaaagtcacgaatctgactttgtccttctaaaggatatatttgaagaaaaacaaattgttcatgaaaatggaacattaaatcatgctgtataaagcaataacaattgattgaccacatgaaagtgtggcctgcactatattctcaattttaatattaagcactcaggagaatgtaggaaagatatcctttgctacagttttgttcagtatctaataagtttgatagatgtattggatacagtactggtttacagaggtttttgtacatttttgagatcattcatgtgtccagagatcttggaaaatattttttcacccacgatttattttgttattgatgatttttttttaaagtggtggtattaagggagagttatctacatggatgagtcttccgctatagcacagtttagaaaaggtgtttatgtcttaattaattgtttgagtacattctttcaacactacacatgaatgaatccaatcttataaccttgaagtgctgtaccagtgctggctgcaggtattaagtccaagtttattaactagatatttatttagtattgagagtaatttgtgaatttgttttgtatttataaaatttatacctgaaaaatgttccttaatgttttaaaccttttactgtgtttttattcctctaacttccttaatgatcaatcaaaaaaagtaacaccctccctttttcctgacagttctttcagctttacagaactgtattataagtttctatgtataactttttaactgtacaaataaaataacattttttcaaataaaaaaaaaaaaaaaaa

SEQ ID NO: 13 - DNA, 1200 bp, Homo sapiens
  gene: Unigene Hs.75939; uridine-cytidine kinase 2 cDNA clone MGC10318 IMAGE3940564
ctccttcgggaaacccagccccgtcaccgggctccgagcggctcgcaggcgagcgacagcggcctcagccccggcagcgcccagcggcggctgcggaaagcggagggagtccgacgcgggcgcgggcggggagcgtgcgtccgttcgcacaggcagcgggaggaggggcggcgcgaaccatggccggggacagcgagcagaccctgcagaaccaccagcagcccaacggcggcgagcccttccttataggcgtcagcgggggaacagctagcggcaagtcttccgtgtgtgctaagatcgtgcagctcctggggcagaatgaggtggactatcgccagaagcaggtggtcatcctgagccaggatagcttctaccgtgtccttacctcggagcagaaggccaaagccctgaagggccagttcaactttgaccacccggatgcctttgacaatgaactcattctcaaaacactcaaagaaatcactgaagggaaaacagtccagatccccgtgtatgactttgtctcccattcccggaaggaggagacagttactgtctatcccgcagacgtggtgctctttgaagggatcctggccttctactcccaggaggtacgagacctgttccagatgaagctttttgtggatacagatgcggacacccggctctcacgcagagtattaagggacatcagcgagagaggcagggatcttgagcagattttatctcagtacattacgttcgtcaagcctgcctttgaggaattctgcttgccaacaaagaagtatgctgatgtgatcatccctagaggtgcagataatctggtggccatcaacctcatcgtgcagcacatccaggacatcctgaatggagggccctccaaacggcagaccaatggctgtctcaacggctacaccccttcacgcaagaggcaggcatcggagtccagcagcaggccgcattgacccgtctccatcggaccccagcccctatctccaagagacagaggaggggtcaggaggcactgctcatctgtacatactgtttcctatgacattactgtatttaagaaaacaccatggagatgaaatgcctttgattttttttttctttttgtactttggaacgacaaaatgaaacagaacttgaccctgagcttaaataacaaaactgtgccaactaaaaaaaaaaaaaaaaaaa

SEQ ID NO: 14 - DNA, 1562 bp, Homo sapiens
  gene: Unigene Hs.75061; MACMARCKS;MARCKS-like 1
gcgcggagcggagcggcggcgggcgcagctagcgggtcggccgcggagcggaggtgcagctcggcttcccccggcacccctccccctcgggcgccagccccacccctccgccggccgggccgaccccgccgtactatcccctgcggcgcgagcccggggcggctccaagcgccccccagcagacccccatcatgggcagccagagctccaaggctccccggggcgacgtgaccgccgaggaggcagcaggcgcttcccccgcgaaggccaacggccaggagaatggccacgtgaaaagcaatggagacttatcccccaagggtgaaggggagtcgccccctgtgaacggaacagatgaggcagccggggccactggcgatgccatcgagccagcaccccctagccagggtgctgaggccaagggggaggtcccccccaaggagacccccaagaagaagaagaaattctctttcaagaagcctttcaaattgagcggcctgtccttcaagagaaatcggaaggagggtgggggtgattcttctgcctcctcacccacagaggaagagcaggagcagggggagatcggtgcctgcagcgacgagggcactgctcaggaagggaaggccgcagccacccctgagagccaggaaccccaggccaagggggcagaggctagtgcagcctcagaagaagaggcagggccccaggctacagagccatccactccctcggggccggagagtggccctacaccagccagcgctgagcagaatgagtagctaggtaggggcaggtgggtgatctctaagctgcaaaaactgtgctgtccttgtgaggtcactgcctggacctggtgccctggctgccttcctgtgcccagaaaggaaggggctattgcctcctcccagccacgttccctttcctcctctccctcctgtggattctcccatcagccatctggttctcctcttaaggccagttgaagatggtcccttacagcttcccaagttaggttagtgatgtgaaatgctcctgtccctggccctacctccttccctgtccccacccctgcataaggcagttgttggttttcttccccaattcttttccaagtaggttttgtttaccctactccccaaatccctgagccagaagtggggtgcttatactcccaaaccttgagtgtccagccttcccctgttgtttttagtctcttgtgctgtgcctagtggcacctgggctggggaggacactgccccgtctaggtttttataaatgtcttactcaagttcaaacctccagcctgtgaatcaactgtgtctcttttttgacttggtaagcaagtattaggctttggggtggggggaggtctgtaatgtgaaacaacttcttgtctttttttctcccactgttgtaaataacttttaatggccaaaccccagatttgtacttttttttttttctaactgctaaaaccattctcttccacctggttttactgtaacatttggaaaaggaataaatgtcgtccctttttaaaaaaaaaaaaaaaaaaaaaaaaa

SEQ ID NO: 15 - DNA, 1090 bp, Homo sapiens
  gene: Unigene Hs.162209; claudin 8 (CLDN8)
ggggcagaatgagatattaaacccaatgctttgattgttctagaaagtatagtaatttgttttctaaggtggttcaagcatctactctttttatcatttacttcaaaatgacattgctaaagactgcattattttactactgtaatttctccacgacatagcattatgtacatagatgagtgtaacatttatatctcacatagagacatgcttatatggttttatttaaaatgaaatgccagtccattacactgaataaatagaactcaactattgcttttcagggaaatcatggatagggttgaagaaggttactattaattgttttaaaaacagcttagggattaatgtcctccatttataatgaagattaaaatgaaggctttaatcagcattgtaaaggaaattgaatggctttctgatatgctgttttttagcctaggagttagaaatcctaacttctttatcctcttctcccagaggctttttttttcttgtgtattaaattaacatttttaaaaagcagatattttgtcaaggggctttgcattcaaactgcttttccagggctatactcagaagaaagataaaagtgtgatctaagaaaaagtgatggttttaggaaagtgaaaatatttttgtttttgtatttgaagaagaatgatgcattttgacaagaaatcatatatgtatggatatattttaataagtatttgagtacagactttgaggtttcatcaatataaataaaagagcagaaaaatatgtcttggttttcatttgcttaccaaaaaaacaacaacaaaaaaagttgtcctttgagaacttcacctgctcctatgtgggtacctgagtcaaaattgtcatttttgttctgtgaaaaataaatttccttcttgtaccatttctgtttagttttactaaaatctgtaaatactgtatttttctgtttattccaaatttgatgaaactgacaatccaatttgaaagtttgtgtcgacgtctgtctagcttaaatgaatgtgttctatttgctttatacatttatattaataaattgtacatttttccaaaaaaaaaaaaaaaaaaa

SEQ ID NO: 16 - DNA, 2208 bp, Homo sapiens
  gene: Unigene Hs.154672; methylenetetrahydrofolate dehydrogenase 2 (MTHFD2)
ggggcctgccacgaggccgcagtataaccgcgtggcccgcgcgcgcgcttccctcccggcgcagtcaccggcgcggtctatggctgcgacttctctaatgtctgctttggctgcccggctgctgcagcccgcgcacagctgctcccttcgccttcgccctttccacctcgcggcagttcgaaatgaagctgttgtcatttctggaaggaaactggcccagcagatcaagcaggaagtgcggcaggaggtagaagagtgggtggcctcaggcaacaaacggccacacctgagtgtgatcctggttggcgagaatcctgcaagtcactcctatgtcctcaacaaaaccagggcagctgcagttgtgggaatcaacagtgagacaattatgaaaccagcttcaatttcagaggaagaattgttgaatttaatcaataaactgaataatgatgataatgtagatggcctccttgttcagttgcctcttccagagcatattgatgagagaaggatctgcaatgctgtttctccagacaaggatgttgatggctttcatgtaattaatgtaggacgaatgtgtttggatcagtattccatgttaccggctactccatggggtgtgtgggaaataatcaagcgaactggcattccaaccctagggaagaatgtggttgtggctggaaggtcaaaaaacgttggaatgcccattgcaatgttactgcacacagatggggcgcatgaacgtcccggaggtgatgccactgttacaatatctcatcgatatactcccaaagagcagttgaagaaacatacaattcttgcagatattgtaatatctgctgcaggtattccaaatctgatcacagcagatatgatcaaggaaggagcagcagtcattgatgtgggaataaatagagttcacgatcctgtaactgccaaacccaagttggttggagatgtggattttgaaggagtcagacaaaaagctgggtatatcactccagttcctggaggtgttggccccatgacagtggcaatgctaatgaagaataccattattgctgcaaaaaaggtgctgaggcttgaagagcgagaagtgctgaagtctaaagagcttggggtagccactaattaactactgtgtcttctgtgtcacaaacagcactccaggccagctcaagaagcaaagcaggccaatagaaatgcaatatttttaatttattctactgaaatggtttaaaatgatgccttgtatttattgaaagcttaaatgggtgggtgtttctgcacatacctctgcagtacctcaccagggagcattccagtatcatgcagggtcctgtgatctagccaggagcagccattaacctagtgattaatatgggagacattaccatatggaggatggatgcttcactttgtcaagcacctcagttacacattcgccttttctaggattgcatttcccaagtgctattgcaataacagttgatactcattttaggtaccaaaccttttgagttcaactgatcaaaccaaaggaaaagtgttgctagagaaaattagggaaaaggtgaaaaagaaaaaatggtagtaattgagcagaaaaaaattaatttatatatgtattgattggcaaccagatttatctaagtagaactgaattggctaggaaaaaagaaaaactgcatgttaatcattttcctaagctgtccttttgaggcttagtcagtttattgggaaaatgtttaggattattccttgctattagtactcattttatgtatgttacccttcagtaagttctccccattttagttttctaggactgaaaggattcttttctacattatacatgtgtgttgtcatatttggcttttgctatatactttaacttcattgttaaatttttgtattgtatagtttctttggtgtatcttaaaacctatttttgaaaaacaaacttggcttgataatcatttgggcagcttgggtaagtacgcaacttacttttccaccaaagaactgtcagcagctgcctgcttttctgtgatgtatgtatcctgttgacttttccagaaattttttaagagtttgagttactattgaatttaatcagactttctgattaaagggttttctttcttttttaataaaacacatctgtctggtatggtatgaatttctgaaaaaaaaaaaaaaaaaaaaaaa

SEQ ID NO: 17 - DNA, 2525 bp, Homo sapiens
  gene: Unigene Hs.18910; solute carrier family 43, member 1 (SLC43A1)
gcttcgcagacctctggcgcccggcgggttcccagttcccccgcttcttccgaggagacagcggaggcgaggccaccgggctgtcaggctgaagctccgtggcggccgggtcctgcacgcagagaagaccccagcgccggcgcggctcagggctgggcccacgggactccggacgcgccgcgaaagcgttgcgctcccggaggcgtccgcagctgctggctgctcatttgccggtgaccggaggctcggggccagcatggcccccacgctgcaacaggcgtaccggaggcgctggtggatggcctgcacggctgtgctggagaacctcttcttctctgctgtactcctgggctggggctccctgttgatcattctgaagaacgagggcttctattccagcacgtgcccagctgagagcagcaccaacaccacccaggatgagcagcgcaggtggccaggctgtgaccagcaggacgagatgctcaacctgggcttcaccattggttccttcgtgctcagcgccaccaccctgccactggggatcctcatggaccgctttggcccccgacccgtgcggctggttggcagtgcctgcttcactgcgtcctgcaccctcatggccctggcctcccgggacgtggaagctctgtctccgttgatattcctggcgctgtccctgaatggctttggtggcatctgcctaacgttcacttcactcacgctgcccaacatgtttgggaacctgcgctccacgttaatggccctcatgattggctcttacgcctcttctgccattacgttcccaggaatcaagctgatctacgatgccggtgtggccttcgtggtcatcatgttcacctggtctggcctggcctgccttatctttctgaactgcaccctcaactggcccatcgaagcctttcctgcccctgaggaagtcaattacacgaagaagatcaagctgagtgggctggccctggaccacaaggtgacaggtgacctcttctacacccatgtgaccaccatgggccagaggctcagccagaaggcccccagcctggaggacggttcggatgccttcatgtcaccccaggatgttcggggcacctcagaaaaccttcctgagaggtctgtccccttacgcaagagcctctgctcccccactttcctgtggagcctcctcaccatgggcatgacccagctgcggatcatcttctacatggctgctgtgaacaagatgctggagtaccttgtgactggtggccaggagcatgagacaaatgaacagcaacaaaaggtggcagagacagttgggttctactcctccgtcttcggggccatgcagctgttgtgccttctcacctgccccctcattggctacatcatggactggcggatcaaggactgcgtggacgccccaactcagggcactgtcctcggagatgccagggacggggttgctaccaaatccatcagaccacgctactgcaagatccaaaagctcaccaatgccatcagtgccttcaccctgaccaacctgctgcttgtgggttttggcatcacctgtctcatcaacaacttacacctccagtttgtgacctttgtcctgcacaccattgttcgaggtttcttccactcagcctgtgggagtctctatgctgcagtgttcccatccaaccactttgggacgctgacaggcctgcagtccctcatcagtgctgtgttcgccttgcttcagcagccacttttcatggcgatggtgggacccctgaaaggagagcccttctgggtgaatctgggcctcctgctattctcactcctgggattcctgttgccttcctacctcttctattaccgtgcccggctccagcaggagtacgccgccaatgggatgggcccactgaaggtgcttagcggctctgaggtgaccgcatagacttctcagaccaagggacctggatgacaggcaatcaaggcctgagcaaccaaaaggagtgccccatatggcttttctacctgtaacatgcacatagagccatggccgtagatttataaataccaagagaagttctatttttgtaaagactgcaaaaaggaggaaaaaaaaccttcaaaaacgccccctaagtcaacgctccattgactgaagacagtccctatcctagaggggttgagctttcttcctccttgggttggaggagaccagggtgcctcttatctccttctagcggtctgcctcctggtacctcttggggggatcggcaaacaggctacccctgaggtcccatgtgccatgagtgtgcacacatgcatgtgtctgtgtatgtgtgaatgtgagagagacacagccctcctttcagaaggaaaggggcctgaggtgccagctgtgtcctgggttaggggttgggggtcggccccttccagggccaggagggcaggttccctctctggtgctgctgcttgcaagtcttagaggaaataaaaagggaagtgagagaaaaaaaaaaaaaaaaaaaaaaaaaaaa

SEQ ID NO: 18 - DNA, 1052 bp, Homo sapiens
  gene: Unigene Hs.109059; mitochondrial ribosomal protein L12 (MRPL12)
gctcgaatgcccggcagccgtggcggctagagcgttcctccccagctcgaatgcccggcggccgaggcggctagagcgtcgcctcctcccggggaaccgcgtgtgaccttccagcccgcggaccgatgctgccggcggccgctcgccccctgtgggggccttgccttgggcttcgggccgctgcgttccgccttgccaggcgacaggtgccatgtgtctgtgccgtgcgacatatgaggagcagcggccatcagaggtgtgaggccctcgctggtgcacccctggataacgcccccaaggagtacccccccaagatacagcagctggtccaggacatcgccagcctcactctcttggaaatctcagacctcaacgagctcctgaagaaaacgttgaagatccaggatgtcgggcttgtgccgatgggtggtgtgatgtctggggctgtccctgctgcagcagcccaggaggcggtggaagaagatatccccatagcgaaagaacggacacatttcaccgtccgcctgaccgaggcgaagcccgtggacaaagtgaagctgatcaaggaaatcaagaactacatccaaggcatcaacctcgtccaggcaaagaagctggtggagtccctgccccaggaaatcaaagccaatgtcgccaaagctgaggcggagaagatcaaggcggccctggaggcggtgggcggcaccgtggttctggagtagcctccagctcggaggacttgtgttcaggggtcctgggccccgggcgaggtcccgccctcccgtggtcactggctccgcccccagcaccaggcgcccagtggagccgtttgggagaattgcctgcgccacgcagcggggccggacaggccgcacagacctactgtggcgggagggaggggcggctgctgcctggtgacggcacccggaggcccaccaggacgcgccaccggtgaatgtgcctctggtggctgctgagaaaaatacactgtgcagctcagaaaaaaaaaaaaaaaaaaaaaaaaaaaaaaaaaaaaaaaaaaaaaaaaaaaa

SEQ ID NO: 19 - protein, 596 aa, Homo sapiens
  PDZ & LIM domain 5 (PDLIM5), transcript variant 1 DKFZp564A072
MSNYSVSLVGPAPWGFRLQGGKDFNMPLTISSLKDGGKAAQANVRIGDVVLSIDGINAQGMTHLEAQNKIKGCTGSLNMTLQRASAAPKPEPVPVQKGEPKEVVKPVPITSPAVSKVTSTNNMAYNKAPRPFGSVSSPKVTSIPSPSSAFTPAHATTSSHASPSPVAAVTPPLFAASGLHANANLSADQSPSALSAGKTAVNVPRQPTVTSVCSETSQELAEGQRRGSQGDSKQQNGPPRKHIVERYTEFYHVPTHSDASKKRLIEDTEDWRPRTGTTQSRSFRILAQITGTEHLKESEADNTKKANNSQEPSPQLASSVASTRSMPESLDSPTSGRPGVTSLTTAAAFKPVGSTGVIKSPSWQRPNQGVPSTGRISNSATYSGSVAPANSALGQTQPSDQDTLVQRAEHIPAGKRTPMCAHCNQVIRGPFLVALGKSWHPEEFNCAHCKNTMAYIGFVEEKGALYCELCYEKFFAPECGRCQRKILGEVINALKQTWHVSCFVCVACGKPIRNNVFHLEDGEPYCETDYYALFGTICHGCEFPIEAGDMFLEALGYTWHDTCFVCSVCCESLEGQTFFSKKDKPLCKKHAHSVNF

SEQ ID NO: 20 - protein, 505 aa, Homo sapiens
  AgX-1 antigen [Homo sapiens]
MNINDLKLTLSKAGQEHLLRFWNELEEAQQVELYAELQAMNFEELNFFFQKAIEGFNQSSHQKNVDARMEPVPREVLGSATRDQDQLQAWESEGLFQISQNKVAVLLLAGGQGTRLGVAYPKGMYDVGLPSRKTLFQIQAERILKLQQVAEKYYGNKCIIPWYIMTSGRTMESTKEFFTKHKYFGLKKENVIFFQQGMLPAMSFDGKIILEEKNKVSMAPDGNGGLYRALAAQNIVEDMEQRGIWSIHVYCVDNILVKVADPRFIGFCIQKGADCGAKVVEKTNPTEPVGVVCRVDGVYQVVEYSEISLATAQKRSSDGRLLFNAGNIANHFFTVPFLRDVVNVYEPQLQHHVAQKKIPYVDTQGQLIKPDKPNGIKMEKFVFDIFQFAKKFVVYEVLREDEFSPLKNADSQNGKDNPTTARHALMSLHHCWVLNAGGHFIDENSSRLPAIPRLKDANDVPIQCEISPLISYAGEGLESYVADKEFHAPLIIDENGVHELVKNGI

SEQ ID NO: 21 - protein, 573 aa, Homo sapiens
  chaperonin [Homo sapiens]
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 22 - protein, 2224 aa, Homo sapiens
  coagulation factor V precursor [Homo sapiens]
MFPGCPRLWVLVVLGTSWVGWGSQGTEAAQLRQFYVAAQGISWSYRPEPTNSSLNLSVTSFKKIVYREYEPYFKKEKPQSTISGLLGPTLYAEVGDIIKVHFKNKADKPLSIHPQGIRYSKLSEGASYLDHTFPAEKMDDAVAPGREYTYEWSISEDSGPTHDDPPCLTHIYYSHENLIEDFNSGLIGPLLICKKGTLTEGGTQKTFDKQIVLLFAVFDESKSWSQSSSLMYTVNGYVNGTMPDITVCAHDHISWHLLGMSSGPELFSIHFNGQVLEQNHHKVSAITLVSATSTTANMTVGPEGKWIISSLTPKHLQAGMQAYIDIKNCPKKTRNLKKITREQRRHMKRWEYFIAAEEVIWDYAPVIPANMDKKYRSQHLDNFSNQIGKHYKKVMYTQYEDESFTKHTVNPNMKEDGILGPIIRAQVRDTLKIVFKNMASRPYSIYPHGVTFSPYEDEVNSSFTSGRNNTMIRAVQPGETYTYKWNILEFDEPTENDAQCLTRPYYSDVDIMRDIASGLIGLLLICKSRSLDRRGIQRAADIEQQAVFAVFDENKSWYLEDNINKFCENPDEVKRDDPKFYESNIMSTINGYVPESITTLGFCFDDTVQWHFCSVGTQNEILTIHFTGHSFIYGKRHEDTLTLFPMRGESVTVTMDNVGTWMLTSMNSSPRSKKLRLKFRDVKCIPDDDEDSYEIFEPPESTVMATRKMHDRLEPEDEESDADYDYQNRLAAALGIRSFRNSSLNQEEEEFNLTALALENGTEFVSSNTDIIVGSNYSSPSNISKFTVNNLAEPQKAPSHQQATTAGSPLRHLIGKNSVLNSSTAEHSSPYSEDPIEDPLQPDVTGIRLLSLGAGEFKSQEHAKHKGPKVERDQAAKHRFSWMKLLAHKVGRHLSQDTGSPSGMRPWEDLPSQDTGSPSRMRPWKDPPSDLLLLKQSNSSKILVGRWHLASEKGSYEIIQDTDEDTAVNNWLISPQNASRAWGESTPLANKPGKQSGHPKFPRVRHKSLQVRQDGGKSRLKKSQFLIKTRKKKKEKHTHHAPLSPRTFHPLRSEAYNTFSERRLKHSLVLHKSNETSLPTDLNQTLPSMDFGWIASLPDHNQNSSNDTGQASCPPGLYQTVPPEEHYQTFPIQDPDQMHSTSDPSHRSSSPELSEMLEYDRSHKSFPTDISQMSPSSEHEVWQTVISPDLSQVTLSPELSQTNLSPDLSHTTLSPELIQRNLSPALGQMPISPDLSHTTLSPDLSHTTLSLDLSQTNLSPELSQTNLSPALGQMPLSPDLSHTTLSLDFSQTNLSPELSHMTLSPELSQTNLSPALGQMPISPDLSHTTLSLDFSQTNLSPELSQTNLSPALGQMPLSPDPSHTTLSLDLSQTNLSPELSQTNLSPDLSEMPLFADLSQIPLTPDLDQMTLSPDLGETDLSPNFGQMSLSPDLSQVTLSPDISDTTLLPDLSQISPPPDLDQIFYPSESSQSLLLQEFNESFPYPDLGQMPSPSSPTLNDTFLSKEFNPLVIVGLSKDGTDYIEIIPKEEVQSSEDDYAEIDYVPYDDPYKTDVRTNINSSRDPDNIAAWYLRSNNGNRRNYYIAAEEISWDYSEFVQRETDIEDSDDIPEDTTYKKVVFRKYLDSTFTKRDPRGEYEEHLGILGPIIRAEVDDVIQVRFKNLASRPYSLHAHGLSYEKSSEGKTYEDDSPEWFKEDNAVQPNSSYTYVWHATERSGPESPGSACRAWAYYSAVNPEKDIHSGLIGPLLICQKGILHKDSNMPMDMREFVLLFMTFDEKKSWYYEKKSRSSWRLTSSEMKKSHEFHAINGMIYSLPGLKMYEQEWVRLHLLNIGGSQDIHVVHFHGQTLLENGNKQHQLGVWPLLPGSFKTLEMKASKPGWWLLNTEVGENQRAGMQTPFLIMDRDCRMPMGLSTGIISDSQIKASEFLGYWEPRLARLNNGGSYNAWSVEKLAAEFASKPWIQVDMQKEVIITGIQTQGAKHYLKSCYTTEFYVAYSSNQINWQIFKGNSTRNVMYFNGNSDASTIKENQFDPPIVARYIRISPTRAYNRPTLRLELQGCEVNGCSTPLGMENGKIENKQITASSFKKSWWGDYWEPFRARLNAQGRVNAWQAKANNNKQWLEIDLLKIKKITAIITQGCKSLSSEMYVKSYTIHYSEQGVEWKPYRLKSSMVDKIFEGNTNTKGHVKNFFNPPIISRFIRVIPKTWNQSIALRLELFGCDIY

SEQ ID NO: 23 - protein, 514 aa, Homo sapiens
  inosine monophosphate dehydrogenase 2 [Homo sapiens]
MADYLISGGTSYVPDDGLTAQQLFNCGDGLTYNDFLILPGYIDFTADQVDLTSALTKKITLKTPLVSSPMDTVTEAGMAIAMALTGGIGFIHHNCTPEFQANEVRKVKKYEQGFITDPVVLSPKDRVRDVFEAKARHGFCGIPITDTGRMGSRLVGIISSRDIDFLKEEEHDCFLEEIMTKREDLVVAPAGITLKEANEILQRSKKGKLPIVNEDDELVAIIARTDLKKNRDYPLASKDAKKQLLCGAAIGTHEDDKYRLDLLAQAGVDVVVLDSSQGNSIFQINMIKYIKDKYPNLQVIGGNVVTAAQAKNLIDAGVDALRVGMGSGSICITQEVLACGRPQATAVYKVSEYARRFGVPVIADGGIQNVGHIAKALALGASTVMMGSLLAATTEAPGEYFFSDGIRLKKYRGMGSLDAMDKHLSSQNRYFSEADKIKVAQGVSGAVQDKGSIHKFVPYLIAGIQHSCQDIGAKSLTQVRAMMYSGELKFEKRTSSAQVEGGVHSLHSYEKRLF

SEQ ID NO: 24 - protein, 216 aa, Homo sapiens
  peptidylprolyl isomerase B precursor [Homo sapiens]
MLRLSERNMKVLLAAALIAGSVFFLLLPGPSAADEKKKGPKVTVKVYFDLRIGDEDVGRVIFGLFGKTVPKTVDNFVALATGEKGFGYKNSKFHRVIKDFMIQGGDFTRGDGTGGKSIYGERFPDENFKLKHYGPGWVSMANAGKDTNGSQFFITTVKTAWLDGKHVVFGKVLEGMEVVRKVESTKTDSRDKPLKDVIIADCGKIEVEKPFAIAKE

SEQ ID NO: 25 - protein, 545 aa, Homo sapiens
  chaperonin containing TCP1, subunit 3 isoform a
MMGHRPVLVLSQNTKRESGRKVQSGNINAAKTIADIIRTCLGPKSMMKMLLDPMGGIVMTNDGNAILREIQVQHPAAKSMIEISRTQDEEVGDGTTSVIILAGEMLSVAEHFLEQQMHPTVVISAYRKALDDMISTLKKISIPVDISDSDMMLNIINSSITTKAISRWSSLACNIALDAVKMVQFEENGRKEIDIKKYARVEKIPGGIIEDSCVLRGVMINKDVTHPRMRRYIKNPRIVLLDSSLEYKKGESQTDIEITREEDFTRILQMEEEYIQQLCEDIIQLKPDVVITEKGISDLAQHYLMRANITAIRRVRKTDNNRIARACGARIVSRPEELREDDVGTGAGLLEIKKIGDEYFTFITDCKDPKACTILLRGASKEILSEVERNLQDAMQVCRNVLLDPQLVPGGGASEMAVAHALTEKSKAMTGVEQWPYRAVAQALEVIPRTLIQNCGASTIRLLTSLRAKHTQENCETWGVNGETGTLVDMKELGIWEPLAVKLQTYKTAVETAVLLLRIDDIVSGHKKKGDDQSRQGGAPDAGQE

SEQ ID NO: 26 - protein, 374 aa, Homo sapiens
  eukaryotic translation initiation factor 3, subunit M
MSVPAFIDISEEDQAAELRAYLKSKGAEISEENSEGGLHVDLAQIIEACDVCLKEDDKDVESVMNSVVSLLLILEPDKQEALIESLCEKLVKFREGERPSLRLQLLSNLFHGMDKNTPVRYTVYCSLIKVAASCGAIQYIPTELDQVRKWISDWNLTTEKKHTLLRLLYEALVDCKKSDAASKVMVELLGSYTEDNASQARVDAHRCIVRALKDPNAFLFDHLLTLKPVKFLEGELIHDLLTIFVSAKLASYVKFYQNNKDFIDSLGLLHEQNMAKMRLLTFMGMAVENKEISFDTMQQELQIGADDVEAFVIDAVRTKMVYCKIDQTQRKVVVSHSTHRTFGKQQWQQLYDTLNAWKQNLNKVKNSLLSLSDT

SEQ ID NO: 27 - protein, 167 aa, Homo sapiens
  regulator of G-protein signaling 10 isoform b
MEHIHDSDGSSSSSHQSLKSTAKWAASLENLLEDPEGVKRFREFLKKEFSEENVLFWLACEDFKKMQDKTQMQEKAKEIYMTFLSSKASSQVNVEGQSRLNEKILEEPHPLMFQKLQDQIFNLMKYDSYSRFLKSDLFLKHKRTEEEEEDLPDAQTAAKRASRIYNT

SEQ ID NO: 28 - protein, 319 aa, Homo sapiens
  pyrroline-5-carboxylate reductase 1 isoform 1
MSVGFIGAGQLAFALAKGFTAAGVLAAHKIMASSPDMDLATVSALRKMGVKLTPHNKETVQHSDVLFLAVKPHIIPFILDEIGADIEDRHIVVSCAAGVTISSIEKKLSAFRPAPRVIRCMTNTPVVVREGATVYATGTHAQVEDGRLMEQLLSSVGFCTEVEEDLIDAVTGLSGSGPAYAFTALDALADGGVKMGLPRRLAVRLGAQALLGAAKMLLHSEQHPGQLKDNVSSPGGATIHALHVLESGGFRSLLINAVEASCIRTRELQSMADQEQVSPAAIKKTILDKVKLDSPAGTALSPSGHTKLLPRSLAPAGKD

SEQ ID NO: 29 - protein, 1556 aa, Homo sapiens
  bromodomain adjacent to zinc finger domain, 1A isoform a
MPLLHRKPFVRQKPPADLRPDEEVFYCKVTNEIFRHYDDFFERTILCNSLVWSCAVTGRPGLTYQEALESEKKARQNLQSFPEPLIIPVLYLTSLTHRSRLHEICDDIFAYVKDRYFVEETVEVIRNNGARLQCRILEVLPPSHQNGFANGHVNSVDGETIIISDSDDSETQSCSFQNGKKKDAIDPLLFKYKVQPTKKELHESAIVKATQISRRKHLFSRDKLKLFLKQHCEPQDGVIKIKASSLSTYKIAEQDFSYFFPDDPPTFIFSPANRRRGRPPKRIHISQEDNVANKQTLASYRSKATKERDKLLKQEEMKSLAFEKAKLKREKADALEAKKKEKEDKEKKREELKKIVEEERLKKKEEKERLKVEREKEREKLREEKRKYVEYLKQWSKPREDMECDDLKELPEPTPVKTRLPPEIFGDALMVLEFLNAFGELFDLQDEFPDGVTLEVLEEALVGNDSEGPLCELLFFFLTAIFQAIAEEEEEVAKEQLTDADTKDLTEALDEDADPTKSALSAVASLAAAWPQLHQGCSLKSLDLDSCTLSEILRLHILASGADVTSANAKYRYQKRGGFDATDDACMELRLSNPSLVKKLSSTSVYDLTPGEKMKILHALCGKLLTLVSTRDFIEDYVDILRQAKQEFRELKAEQHRKEREEAAARIRKRKEEKLKEQEQKMKEKQEKLKEDEQRNSTADISIGEEEREDFDTSIESKDTEQKELDQDMVTEDEDDPGSHKRGRRGKRGQNGFKEFTRQEQINCVTREPLTADEEEALKQEHQRKEKELLEKIQSAIACTNIFPLGRDRMYRRYWIFPSIPGLFIEEDYSGLTEDMLLPRPSSFQNNVQSQDPQVSTKTGEPLMSESTSNIDQGPRDHSVQLPKPVHKPNRWCFYSSCEQLDQLIEALNSRGHRESALKETLLQEKSRICAQLARFSEEKFHFSDKPQPDSKPTYSRGRSSNAYDPSQMCAEKQLELRLRDFLLDIEDRIYQGTLGAIKVTDRHIWRSALESGRYELLSEENKENGIIKTVNEDVEEMEIDEQTKVIVKDRLLGIKTETPSTVSTNASTPQSVSSVVHYLAMALFQIEQGIERRFLKAPLDASDSGRSYKTVLDRWRESLLSSASLSQVFLHLSTLDRSVIWSKSILNARCKICRKKGDAENMVLCDGCDRGHHTYCVRPKLKTVPEGDWFCPECRPKQRSRRLSSRQRPSLESDEDVEDSMGGEDDEVDGDEEEGQSEEEEYEVEQDEDDSQEEEEVSLPKRGRPQVRLPVKTRGKLSSSFSSRGQQQEPGRYPSRSQQSTPKTTVSSKTGRSLRKINSAPPTETKSLRIASRSTRHSHGPLQADVFVELLSPRRKRRGRKSANNTPENSPNFPNFRVIATKSSEQSRSVNIASKLSLQESESKRRCRKRQSPEPSPVTLGRRSSGRQGGVHELSAFEQLVVELVRHDDSWPFLKLVSKIQVPDYYDIIKKPIALNIIREKVNKCEYKLASEFIDDIELMFSNCFEYNPRNTSEAKAGTRLQAFFHIQAQKLGLHVTPSNVDQVSTPPAAKKSRI

SEQ ID NO: 30 - protein, 195 aa, Homo sapiens
  MARCKS-like 1
MGSQSSKAPRGDVTAEEAAGASPAKANGQENGHVKSNGDLSPKGEGESPPVNGTDEAAGATGDAIEPAPPSQGAEAKGEVPPKETPKKKKKFSFKKPFKLSGLSFKRNRKEGGGDSSASSPTEEEQEQGEIGACSDEGTAQEGKAAATPESQEPQAKGAEASAASEEEAGPQATEPSTPSGPESGPTPASAEQNE

SEQ ID NO: 31 - protein, 350 aa, Homo sapiens
  methylene tetrahydrofolate dehydrogenase 2 isoform A precursor
MAATSLMSALAARLLQPAHSCSLRLRPFHLAAVRNEAVVISGRKLAQQIKQEVRQEVEEWVASGNKRPHLSVILVGENPASHSYVLNKTRAAAVVGINSETIMKPASISEEELLNLINKLNNDDNVDGLLVQLPLPEHIDERRICNAVSPDKDVDGFHVINVGRMCLDQYSMLPATPWGVWEIIKRTGIPTLGKNVVVAGRSKNVGMPIAMLLHTDGAHERPGGDATVTISHRYTPKEQLKKHTILADIVISAAGIPNLITADMIKEGAAVIDVGINRVHDPVTAKPKLVGDVDFEGVRQKAGYITPVPGGVGPMTVAMLMKNTIIAAKKVLRLEEREVLKSKELGVATN

SEQ ID NO: 32 - protein, 559 aa, Homo sapiens
  solute carrier family 43, member 1
MAPTLQQAYRRRWWMACTAVLENLFFSAVLLGWGSLLIILKNEGFYSSTCPAESSTNTTQDEQRRWPGCDQQDEMLNLGFTIGSFVLSATTLPLGILMDRFGPRPVRLVGSACFTASCTLMALASRDVEALSPLIFLALSLNGFGGICLTFTSLTLPNMFGNLRSTLMALMIGSYASSAITFPGIKLIYDAGVAFVVIMFTWSGLACLIFLNCTLNWPIEAFPAPEEVNYTKKIKLSGLALDHKVTGDLFYTHVTTMGQRLSQKAPSLEDGSDAFMSPQDVRGTSENLPERSVPLRKSLCSPTFLWSLLTMGMTQLRIIFYMAAVNKMLEYLVTGGQEHETNEQQQKVAETVGFYSSVFGAMQLLCLLTCPLIGYIMDWRIKDCVDAPTQGTVLGDARDGVATKSIRPRYCKIQKLTNAISAFTLTNLLLVGFGITCLINNLHLQFVTFVLHTIVRGFFHSACGSLYAAVFPSNHFGTLTGLQSLISAVFALLQQPLFMAMVGPLKGEPFWVNLGLLLFSLLGFLLPSYLFYYRARLQQEYAANGMGPLKVLSGSEVTA

SEQ ID NO: 33 - protein, 198 aa, Homo sapiens [sequence omitted]

(19) United States
(12) Patent Application Publication
(10) Pub. No.: US 2009/0215024 A1
(43) Pub. Date: Aug. 27, 2009

(54) BIOMARKERS UPREGULATED IN PROSTATE CANCER

(75) Inventor: ISABELLE GUYON, BERKELEY, CA (US)

Correspondence Address: PROCOPIO, CORY, HARGREAVES & SAVITCH LLP, 530 B STREET, SUITE 2100, SAN DIEGO, CA, 92101, US

(73) Assignee: HEALTH DISCOVERY CORPORATION, Savannah, GA (US)

(21) Appl. No.: 12/025,724

(22) Filed: Feb. 4, 2008

Related U.S. Application Data
(63) Continuation-in-part of application No. 11/274,931, filed on Nov. 14, 2005, now abandoned.
Application No. 11/274,931 is a continuation-in-part of application No. 10/057,849, filed on Jan. 24, 2002, now Pat. No. 7,117,188.
(60) Provisional application No. 60/888,070, filed on Feb. 2, 2007.
(60) Provisional application No. 60/627,626, filed on Nov. 12, 2004.
(60) Provisional application No. 60/651,340, filed on Feb. 9, 2005.
(60) Provisional application No. 60/263,696, filed on Jan. 24, 2001.
(60) Provisional application No. 60/298,757, filed on Jun. 15, 2001.
(60) Provisional application No. 60/275,760, filed on Mar. 14, 2001.

(51) Int. Cl. C12Q 1/00

(52) U.S. Cl. 435/4

(57) ABSTRACT

Biomarkers are identified by analyzing gene expression data using support vector machines (SVM) to rank genes according to their ability to separate prostate cancer from normal tissue. Proteins expressed by identified genes are detected in patient samples to screen, predict and monitor prostate cancer.

### RELATED APPLICATIONS

The present application claims priority to U.S. provisional Application No. 60/880,070, filed Feb. 2, 2007, and is a continuation-in-part of U.S. application Ser. No. 11/274,931, filed Nov. 14, 2005, which claims priority to each of U.S. Provisional Applications No. 60/627,626, filed Nov. 12, 2004, and No. 60/651,340, filed Feb. 9, 2005, and is a continuation-in-part of U.S. application Ser. No. 10/057,849, now issued as U.S. Pat. No. 7,117,188, which claims priority to each of U.S. Provisional Applications No. 60/263,696, filed Jan. 24, 2001, No. 60/298,757, filed Jun. 15, 2001, and No. 60/275,760, filed Mar. 14, 2001, and is a continuation-in-part of U.S. patent application Ser. No. 09/633,410, filed Aug. 7, 2000, now issued as U.S. Pat. No. 6,882,990, which claims priority to each of U.S. Provisional Applications No. 60/161,806, filed Oct. 27, 1999, No. 60/168,703, filed Dec. 2, 1999, No. 60/184,596, filed Feb. 24, 2000, No. 60/191,219, filed Mar. 22, 2000, and No. 60/207,026, filed May 25, 2000. Each of the above cited applications and patents is incorporated herein by reference.

### FIELD OF THE INVENTION

The present invention relates to the use of learning machines to identify relevant patterns in datasets containing large quantities of gene expression data, and more particularly to biomarkers so identified for use in screening, predicting, and monitoring prostate cancer.

### BACKGROUND OF THE INVENTION

Knowledge discovery is the most desirable end product of data collection. Recent advancements in database technology have lead to an explosive growth in systems and methods for generating, collecting and storing vast amounts of data. While database technology enables efficient collection and storage of large data sets, the challenge of facilitating human comprehension of the information in this data is growing ever more difficult. With many existing techniques the problem has become unapproachable. In particular, methods are needed for identifying patterns in biological systems as reflected in gene expression data.

A significant percentage of men (20%) in the U.S. are diagnosed with prostate cancer during their lifetime, with nearly 300,000 men diagnosed annually, a rate second only to skin cancer. However, only 3% of those die of the disease. About 70% of all diagnosed prostate cancers occur in men aged 65 years and older. Many prostate cancer patients have undergone aggressive treatments that can have life-altering side effects such as incontinence and sexual dysfunction. It is believed that a substantial portion of the cancers are over-treated. Currently, most early prostate cancer identification is done using prostate-specific antigen (PSA) screening, but few indicators currently distinguish between progressive prostate tumors that may metastasize and escape local treatment and indolent cancers of benign prostate hyperplasia (BPH). Further, some studies have shown that PSA is a poor predictor of cancer, instead tending to predict BPH, which requires no or little treatment.

There is an urgent need for new biomarkers for distinguishing between normal, benign and malignant prostate tissue and for predicting the size and malignancy of prostate cancer. Blood serum biomarkers, or biomarkers found in semen, would be particularly desirable for screening prior to biopsy, however, evaluation of gene expression microarrays from biopsied prostate tissue is also useful.

### SUMMARY OF THE INVENTION

Gene expression data are analyzed using learning machines such as support vector machines (SVM) and ridge regression classifiers to rank genes according to their ability to separate prostate cancer from other prostate conditions including BPH and normal. Genes are identified that individually provide sensitivities and selectivities of better than 80% and, when combined in small groups, 90%, for separating prostate cancer from other prostate conditions.

An exemplary embodiment comprises methods and systems for detecting genes involved with prostate cancer and determination of methods and compositions for treatment of prostate cancer. In one embodiment, to improve the statistical significance of the results, supervised learning techniques can analyze data obtained from a number of different sources using different microarrays, such as the Affymetrix U95 and U133A GeneChip® chip sets.

### BRIEF DESCRIPTION OF THE DRAWINGS

FIG. 1 is a functional block diagram illustrating an exemplary operating environment for an embodiment of the present invention.

FIG. 2 is a plot showing the results based on LCM data preparation for prostate cancer analysis.

FIG. 3 is a plot graphically comparing SVM-RFE of the present invention with leave-one-out classifier for prostate cancer.

FIGS. 4a-4d combined are a table showing the ranking of the top 200 genes for separating prostate tumor from other tissues.

FIGS. 5a-5o combined are two tables showing the top 200 genes for separating prostate cancer from all other tissues that were identified in each of the 2001 study and the 2003 study.

FIGS. 6a-6g combined are a table showing the top 200 genes for separating G3 and G4 tumor versus others using feature ranking by consensus between the 2001 study and the 2003 study.

FIG. 7 is a plot of performance as a function of number of genes selected.

FIG. 8 is a plot of the ROC curves for the 3 top RFE selected genes and the ROC of the combination, on test data.

FIG. 9 is a prior art diagram showing the KEGG pathway around gene AgX-1/UAP 1/SPAG2.

FIG. 10 is a dendogram showing gene expression clustering of mitochondrial genes.

FIG. 11 is a dendogram showing gene expression clustering of perixosome and cell adhesion genes.

FIG. 12 is a dendogram showing gene expression clustering of genes linked to cell proliferation and growth.

FIG. 13 is a dendogram showing gene expression clustering of genes linked to apoptosis or p53 pathway.

### DETAILED DESCRIPTION OF THE PREFERRED EMBODIMENT

The present invention utilizes learning machine techniques, including support vector machines and ridge regression, to discover knowledge from gene expression data obtained by measuring hybridization intensity of gene and gene fragment probes on microarrays. The knowledge so discovered can be used for diagnosing and prognosing changes in biological systems, such as diseases. Preferred embodiments comprise identification of genes that will distinguish between different types of prostate disorders, such as benign prostate hyperplasy and cancer, and normal, and use of such information for decisions on treatment of patients with prostate disorders.

For purposes of the present invention, “gene” refers to the gene expression products corresponding to genes, gene fragments, ESTs and olionucleotides that are included on the Affymetrix microarrays used in the tests described in the examples. Identification of a gene by a GeneBank accession number (GAN), Unigene No. and/or gene name constitutes an express incorporation by reference of the record corresponding to that identifier in the National Center for Biotechnology Information (NCBI) databases, which is publicly accessible and well known to those of skill in the art.

The problem of selection of a small amount of data from a large data source, such as a gene subset from a microarray, is particularly solved using the methods described herein. Preferred methods described herein use support vector machine (SVM) methods based and recursive feature elimination (RFE), which is described in detail in U.S. Pat. No. 7,117,188, which is incorporated by reference. (It should be noted that “RFE-SVM” and “SVM-RFE” may be used interchangeably throughout the detailed description, however, both refer to the same technique.) In examining gene expression data to find determinative genes, these methods eliminate gene redundancy automatically and yield better and more compact gene subsets.

The data is input into computer system programmed for executing an algorithm using a learning machine for performing a feature selection and/or ranking, preferably a SVM-RFE. The SVM-RFE is run one or more times to generate the best feature selections, which can be displayed in an observation graph or listed in a table or other display format. (Examples of listings of selected features (in this case, genes) are included in many of the tables below.) The SVM may use any algorithm and the data may be preprocessed and postprocessed if needed. Preferably, a server contains a first observation graph that organizes the results of the SVM activity and selection of features.

The information generated by the SVM may be examined by outside experts, computer databases, or other complementary information sources. For example, if the resulting feature selection information is about selected genes, biologists or experts or computer databases may provide complementary information about the selected genes, for example, from medical and scientific literature. Using all the data available, the genes are given objective or subjective grades. Gene interactions may also be recorded.

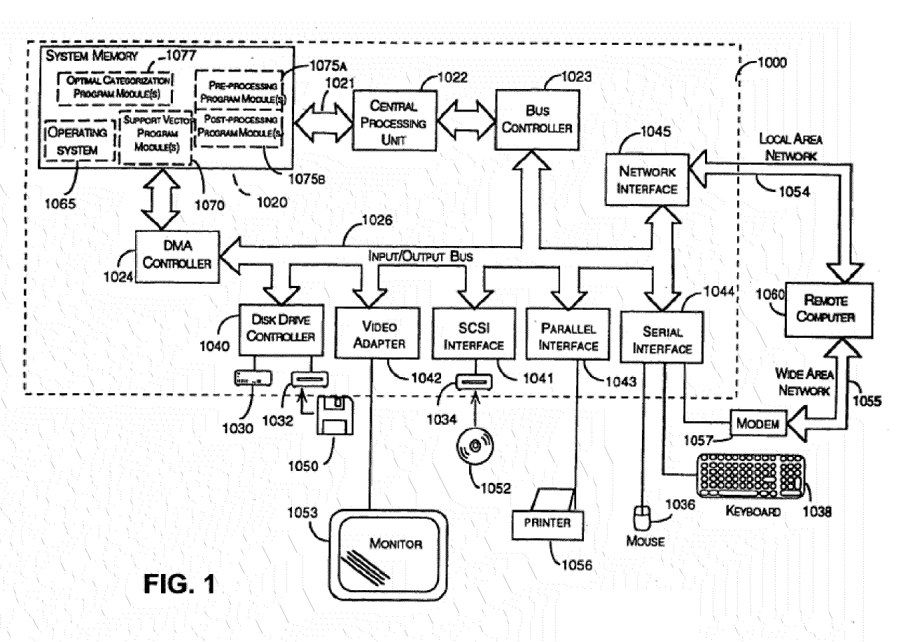

FIG. 1 and the following discussion are intended to provide a brief and general description of a suitable computing environment for implementing biological data analysis according to the present invention. Although the system shown in FIG. 1 is a conventional personal computer 1000, those skilled in the art will recognize that the invention also may be implemented using other types of computer system configurations. The computer 1000 includes a central processing unit 1022, a system memory 1020, and an Input/Output (“I/O”) bus 1026. A system bus 1021 couples the central processing unit 1022 to the system memory 1020. A bus controller 1023 controls the flow of data on the I/O bus 1026 and between the central processing unit 1022 and a variety of internal and external I/O devices. The I/O devices connected to the I/O bus 1026 may have direct access to the system memory 1020 using a Direct Memory Access (“DMA”) controller 1024.

The I/O devices are connected to the I/O bus 1026 via a set of device interfaces. The device interfaces may include both hardware components and software components. For instance, a hard disk drive 1030 and a floppy disk drive 1032 for reading or writing removable media 1050 may be connected to the I/O bus 1026 through disk drive controllers 1040. An optical disk drive 1034 for reading or writing optical media 1052 may be connected to the I/O bus 1026 using a Small Computer System Interface (“SCSI”) 1041. Alternatively, an IDE (Integrated Drive Electronics, i.e., a hard disk drive interface for PCs), ATAPI (ATtAchment Packet Interface, i.e., CD-ROM and tape drive interface), or EIDE (Enhanced IDE) interface may be associated with an optical drive such as may be the case with a CD-ROM drive. The drives and their associated computer-readable media provide nonvolatile storage for the computer 1000. In addition to the computer-readable media described above, other types of computer-readable media may also be used, such as ZIP drives, or the like.

A display device 1053, such as a monitor, is connected to the I/O bus 1026 via another interface, such as a video adapter 1042. A parallel interface 1043 connects synchronous peripheral devices, such as a laser printer 1056, to the I/O bus 1026. A serial interface 1044 connects communication devices to the I/O bus 1026. A user may enter commands and information into the computer 1000 via the serial interface 1044 or by using an input device, such as a keyboard 1038, a mouse 1036 or a modem 1057. Other peripheral devices (not shown) may also be connected to the computer 1000, such as audio input/output devices or image capture devices.

A number of program modules may be stored on the drives and in the system memory 1020. The system memory 1020 can include both Random Access Memory (“RAM”) and Read Only Memory (“ROM”). The program modules control how the computer 1000 functions and interacts with the user, with I/O devices or with other computers. Program modules include routines, operating systems 1065, application programs, data structures, and other software or firmware components. In an illustrative embodiment, the learning machine may comprise one or more pre-processing program modules 1075A, one or more post-processing program modules 1075B, and/or one or more optimal categorization program modules 1077 and one or more SVM program modules 1070 stored on the drives or in the system memory 1020 of the computer 1000. Specifically, pre-processing program modules 1075A, post-processing program modules 1075B, together with the SVM program modules 1070 may comprise computer-executable instructions for pre-processing data and post-processing output from a learning machine and implementing the learning algorithm. Furthermore, optimal categorization program modules 1077 may comprise computer-executable instructions for optimally categorizing a data set.

The computer 1000 may operate in a networked environment using logical connections to one or more remote computers, such as remote computer 1060. The remote computer 1060 may be a server, a router, a peer to peer device or other common network node, and typically includes many or all of the elements described in connection with the computer 1000. In a networked environment, program modules and data may be stored on the remote computer 1060. Appropriate logical connections include a local area network (“LAN”) and a wide area network (“WAN”). In a LAN environment, a network interface, such as an Ethernet adapter card, can be used to connect the computer to the remote computer. In a WAN environment, the computer may use a telecommunications device, such as a modem, to establish a connection. It will be appreciated that the network connections shown are illustrative and other devices of establishing a communications link between the computers may be used.

A preferred selection browser is preferably a graphical user interface that would assist final users in using the generated information. For example, in the examples herein, the selection browser is a gene selection browser that assists the final user is selection of potential drug targets from the genes identified by the SVM RFE. The inputs are the observation graph, which is an output of a statistical analysis package and any complementary knowledge base information, preferably in a graph or ranked form. For example, such complementary information for gene selection may include knowledge about the genes, functions, derived proteins, measurement assays, isolation techniques, etc. The user interface preferably allows for visual exploration of the graphs and the product of the two graphs to identify promising targets. The browser does not generally require intensive computations and if needed, can be run on other computer means. The graph generated by the server can be precomputed, prior to access by the browser, or is generated in situ and functions by expanding the graph at points of interest.

In a preferred embodiment, the server is a statistical analysis package, and in the gene feature selection, a gene selection server. For example, inputs are patterns of gene expression, from sources such as DNA microarrays or other data sources. Outputs are an observation graph that organizes the results of one or more runs of SVM RFE. It is optimum to have the selection server run the computationally expensive operations.

A preferred method of the server is to expand the information acquired by the SVM. The server can use any SVM results, and is not limited to SVM RFE selection methods. As an example, the method is directed to gene selection, though any data can be treated by the server. Using SVM RFE for gene selection, gene redundancy is eliminated, but it is informative to know about discriminant genes that are correlated with the genes selected. For a given number N of genes, only one combination is retained by SVM-RFE. In actuality, there are many combinations of N different genes that provide similar results.

A combinatorial search is a method allowing selection of many alternative combinations of N genes, but this method is prone to overfitting the data. SVM-RFE does not overfit the data. SVM-RFE is combined with supervised clustering to provide lists of alternative genes that are correlated with the optimum selected genes. Mere substitution of one gene by another correlated gene yields substantial classification performance degradation.

The examples included herein show preferred methods for determining the genes that are most correlated to the presence of cancer or can be used to predict cancer occurrance in an individual. There is no limitation to the source of the data and the data can be combinations of measurable criteria, such as genes, proteins or clinical tests, that are capable of being used to differentiate between normal conditions and changes in conditions in biological systems.

In the following examples, preferred numbers of genes were determined that result from separation of the data that discriminate. These numbers are not limiting to the methods of the present invention. Preferably, the preferred optimum number of genes is a range of approximately from 1 to 500, more preferably, the range is from 10 to 250, from 1 to 50, even more preferably the range is from 1 to 32, still more preferably the range is from 1 to 21 and most preferably, from 1 to 10. The preferred optimum number of genes can be affected by the quality and quantity of the original data and thus can be determined for each application by those skilled in the art.

Once the determinative genes are found by the learning machines of the present invention, methods and compositions for treatments of the biological changes in the organisms can be employed. For example, for the treatment of cancer, therapeutic agents can be administered to antagonize or agonize, enhance or inhibit activities, presence, or synthesis of the gene products. Therapeutic agents and methods include, but are not limited to, gene therapies such as sense or antisense polynucleotides, DNA or RNA analogs, pharmaceutical agents, plasmaphoresis, antiangiogenics, and derivatives, analogs and metabolic products of such agents.

Such agents may be administered via parenteral or noninvasive routes. Many active agents are administered through parenteral routes of administration, intravenous, intramuscular, subcutaneous, intraperitoneal, intraspinal, intrathecal, intracerebroventricular, intraarterial and other routes of injection. Noninvasive routes for drug delivery include oral, nasal, pulmonary, rectal, buccal, vaginal, transdermal and occular routes.

The following examples illustrate the results of using SVMs and other learning machines to identify genes associated with disorders of the prostate. Such genes may be used for diagnosis, treatment, in terms of identifying appropriate therapeutic agents, and for monitoring the progress of treatment.

### Example 1

### Isolation of Genes Involved with Prostate Cancer

Using the methods disclosed herein, genes associated with prostate cancer were isolated. Various methods of treating and analyzing the cells, including SVM, were utilized to determine the most reliable method for analysis.

Tissues were obtained from patients that had cancer and had undergone prostatectomy. The tissues were processed according to a standard protocol of Affymetrix and gene expression values from 7129 probes on the Affymetrix U95 GeneChip® were recorded for 67 tissues from 26 patients.

Specialists of prostate histology recognize at least three different zones in the prostate: the peripheral zone (PZ), the central zone (CZ), and the transition zone (TZ). In this study, tissues from all three zones are analyzed because previous findings have demonstrated that the zonal origin of the tissue is an important factor influencing the genetic profiling. Most prostate cancers originate in the PZ. Cancers originating in the PZ have worse prognosis than those originating in the TZ. Contemporary biopsy strategies concentrate on the PZ and largely ignore cancer in the TZ. Benign prostate hyperplasia (BPH) is found only in the TZ. BPH is a suitable control that may be used to compare cancer tissues in genetic profiling experiments. BPH is also convenient to use as control because it is abundant and easily dissected. However, controls coming from normal tissues microdissected with lasers in the CZ and PZ can also provide important complementary controls. The gene expression profile differences have been found to be larger between PZ-G4-G5 cancer and CZ-normal used as control, compared to PZ-normal used as control. A possible explanation comes from the fact that is presence of cancer, even normal adjacent tissues have undergone DNA changes (Malins et al, 2003-2004). Table 1 gives zone properties.

TABLE 1

| Zone | Properties |
|---|---|
| PZ | From apex posterior to base, surrounds transition and central zones. |
| | Largest zone (70% in young men). |
| | Largest number cancers (60-80%). |
| | Dysplasia and atrophy common in older men. |
| CZ | Surrounds transition zone to angle of urethra to bladder base. |
| | Second largest zone (25% in young men to 30% at 40 year old). |
| | 50% of PSA secreting epithelium. |
| | 5-20% of cancers. |
| TZ | Two pear shaped lobes surrounding the proximal urethra. |
| | Smallest zone in young men (less than 5%). |
| | Gives rise to BPH in older men. May expand to the bulk of the |
| | gland. |
| | 10-18% of cancers. |
| | Better cancer prognosis than PZ cancer. |

Classification of cancer determines appropriate treatment and helps determine a prognosis. Cancer develops progressively from an alteration in a cell's genetic structure due to mutations, to cells with uncontrolled growth patterns. Classification is made according to the site of origin, histology (or cell analysis; called grading), and the extent of the disease (called staging).

Prostate cancer specialists classify cancer tissues according to grades, called Gleason grades, which are correlated with the malignancy of the diseases. The larger the grade, the worse the prognosis (chance of survival). In this study, tissues of grade 3 and above are used. Grades 1 and 2 are more difficult to characterize with biopsies and not very malignant. Grades 4 and 5 are not very differentiated and correspond to the most malignant cancers: for every 10% increase in the percent of grade 4/5 tissue found, there is a concomitant increase in post radical prostatectomy failure rate. Each grade is defined in Table 2.

TABLE 2

| Grade | Description |
|---|---|
| 1 | Single, separate, uniform, round glands closely packed with a definite rounded |
| | edge limiting the area of the tumor. Separation of glands at the periphery from the |
| | main collection by more than one gland diameter indicates a component of at least |
| | grade 2. Uncommon pattern except in the TZ. Almost never seen in needle |
| | biopsies. |
| 2 | Like grade 1 but more variability in gland shape and more stroma separating |
| | glands. Occasional glands show angulated or distorted contours. More common |
| | in TZ than PZ. Pathologists don't diagnose Gleason grades 1 or 2 on prostate |
| | needle biopsies since they are uncommon in the PZ, there is inter-pathologist |
| | variability and poor correlation with radical prostatectomy. |
| 3 | G3 is the most commonly seen pattern. Variation in size, shape (may be |
| | angulated or compressed), and spacing of glands (may be separated by >1 gland |
| | diameter). Many small glands have occluded or abortive lumens (hollow areas). |
| | There is no evidence of glandular fusion. The malignant glands infiltrate between |
| | benign glands. |
| 4 | The glands are fused and there is no intervening stroma. |
| 5 | Tumor cells are arranged in solid sheets with no attempts at gland formation. The |
| | presence of Gleason grade 5 and high percent carcinoma at prostatectomy predicts |
| | early death. |

Staging is the classification of the extent of the disease. There are several types of staging methods. The tumor, node, metastases (TNM) system classifies cancer by tumor size (T), the degree of regional spread or lymph node involvement (N), and distant metastasis (M). The stage is determined by the size and location of the cancer, whether it has invaded the prostatic capsule or seminal vesicle, and whether it has metastasized. For staging, MRI is preferred to CT because it permits more accurate T staging. Both techniques can be used in N staging, and they have equivalent accuracy. Bone scintigraphy is used in M staging.

The grade and the stage correlate well with each other and with the prognosis. Adenocarcinomas of the prostate are given two grade based on the most common and second most common architectural patterns. These two grades are added to get a final score of 2 to 10. Cancers with a Gleason score of <6 are generally low grade and not aggressive.

The samples collected included tissues from the Peripheral Zone (PZ); Central Zone (CZ) and Transition Zone (TZ). Each sample potentially consisted of four different cell types: Stomal cells (from the supporting tissue of the prostate, not participating in its function); Normal organ cells; Benign prostatic hyperplasia cells (BPH); Dysplasia cells (cancer precursor stage) and Cancer cells (of various grades indicating the stage of the cancer). The distribution of the samples in Table 3 reflects the difficulty of obtaining certain types of tissues:

TABLE 3

| | | | | | Cancer | Cancer | |
|---|---|---|---|---|---|---|---|
| | Stroma | Normal | BPH | Dysplasia | G3 | G4 | G3 + G4 |
| PZ | 1 | 5 | | 3 | 10 | 24 | 3 |
| CZ | | 3 | | | | | |
| TZ | | | 18 | | | | |

Benign Prostate Hyperplasia (BPH), also called nodular prostatic hyperplasia, occurs frequently in aging men. By the eighth decade, over 90% of males will have prostatic hyperplasia. However, in only a minority of cases (about 10%) will this hyperplasia be symptomatic and severe enough to require surgical or medical therapy. BPH is not a precursor to carcinoma.

It has been argued in the medical literature that TZ BPH could serve as a good reference for PZ cancer. The highest grade cancer (G4) is the most malignant. Part of these experiments are therefore directed towards the separation of BPH vs. G4.

Some of the cells were prepared using laser confocal microscopy (LCM which was used to eliminate as much of the supporting stromal cells as possible and provides purer samples.

Gene expression was assessed from the presence of mRNA in the cells. The mRNA is converted into cDNA and amplified, to obtain a sufficient quantity. Depending on the amount of mRNA that can be extracted from the sample, one or two amplifications may be necessary. The amplification process may distort the gene expression pattern. In the data set under study, either 1 or 2 amplifications were used. LCM data always required 2 amplifications. The treatment of the samples is detailed in Table 4.

TABLE 4

| | 1 amplification | 2 amplifications |
|---|---|---|
| No LCM | 33 | 14 |
| LCM | | 20 |

The end result of data extraction is a vector of 7129 gene expression coefficients.

Gene expression measurements require calibration. A probe cell (a square on the array) contains many replicates of the same oligonucleotide (probe) that is a 25 bases long sequence of DNA. Each “perfect match” (PM) probe is designed to complement a reference sequence (piece of gene). It is associated with a “mismatch” (MM) probe that is identical except for a single base difference in the central position. The chip may contain replicates of the same PM probe at different positions and several MM probes for the same PM probe corresponding to the substitution of one of the four bases. This ensemble of probes is referred to as a probe set. The gene expression is calculated as:

Average Difference=1/pair num Σ_{probe set}(PM−MM)

If the magnitude of the probe pair values is not sufficiently contrasted, the probe pair is considered dubious. Thresholds are set to accept or reject probe pairs. Affymetrix considers samples with 40% or over acceptable probe pairs of good quality. Lower quality samples can also be effectively used with the SVM techniques.

A simple “whitening” was performed as pre-processing, so that after pre-processing, the data matrix resembles “white noise”. In the original data matrix, a line of the matrix represented the expression values of 7129 genes for a given sample (corresponding to a particular combination of patient/tissue/preparation method). A column of the matrix represented the expression values of a given gene across the 67 samples. Without normalization, neither the lines nor the columns can be compared. There are obvious offset and scaling problems. The samples were pre-processed to: normalize matrix columns; normalize matrix lines; and normalize columns again. Normalization consists of subtracting the mean and dividing by the standard deviation. A further normalization step was taken when the samples are split into a training set and a test set.

The mean and variance column-wise was computed for the training samples only. All samples (training and test samples) were then normalized by subtracting that mean and dividing by the standard deviation.

Samples were evaluated to determine whether LCM data preparation yields more informative data than unfiltered tissue samples and whether arrays of lower quality contain useful information when processed using the SVM technique.

Two data sets were prepared, one for a given data preparation method (subset 1) and one for a reference method (subset 2). For example, method 1=LCM and method 2=unfiltered samples. Golub's linear classifiers were then trained to distinguish between cancer and normal cases using subset 1 and another classifier using subset 2. The classifiers were then tested on the subset on which they had not been trained (classifier 1 with subset 2 and classifier 2 with subset 1).

If classifier 1 performs better on subset 2 than classifier 2 on subset 1, it means that subset 1 contains more information to do the separation cancer vs. normal than subset 2.

The input to the classifier is a vector of n “features” that are gene expression coefficients coming from one microarray experiment. The two classes are identified with the symbols (+) and (−) with “normal” or reference samples belong to class (+) and cancer tissues to class (−). A training set of a number of patterns {x₁, x₂, . . . xₖ, . . . , xₗ} with known class labels {y₁, y₂, . . . yₖ, . . . yₗ}, yₖ∈{−1,+1}, is given. The training samples are used to build a decision function (or discriminant function) D(x), that is a scalar function of an input pattern x. New samples are classified according to the sign of the decision function:

D(x)>0 [symbol] ×∈class (+)

D(x)<0 [symbol] ×∈class (−)

D(x)=0, decision boundary.

Decision functions that are simple weighted sums of the training patterns plus a bias are called linear discriminant functions.

D(x)=w·x+b,

where w is the weight vector and b is a bias value.

In the case of Golub's classifier, each weight is computed as:

Wᵢ=(μᵢ(+)−μᵢ(−))/(σᵢ(+)+σᵢ(−)),

where (μᵢ and σᵢ are the mean and standard deviation of the gene expression values of gene i for all the patients of class (+) or class (−), i=1, . . . n. Large positive wᵢ values indicate strong correlation with class (+) whereas large negative wᵢ values indicate strong correlation with class (−). Thus the weights can also be used to rank the features (genes) according to relevance. The bias is computed as b=−w·μ, where μ=(μ(+)+(−))/2.

Golub's classifier is a standard reference that is robust against outliers. Once a first classifier is trained, the magnitude of wᵢ is used to rank the genes. The classifiers are then retrained with subsets of genes of different sizes, including the best ranking genes.

To assess the statistical significance of the results, ten random splits of the data including samples were prepared from either preparation method and submitted to the same method. This allowed the computation of an average and standard deviation for comparison purposes.

Tissue from the same patient was processed either directly (unfiltered) or after the LCM procedure, yielding a pair of microarray experiments. This yielded 13 pairs, including: four G4; one G3+4; two G3; four BPH; one CZ (normal) and one PZ (normal).

For each data preparation method (LCM or unfiltered tissues), the tissues were grouped into two subsets:

Cancer=G4+G3 (7 cases)

Normal=BPH+CZ+PZ (6 cases).

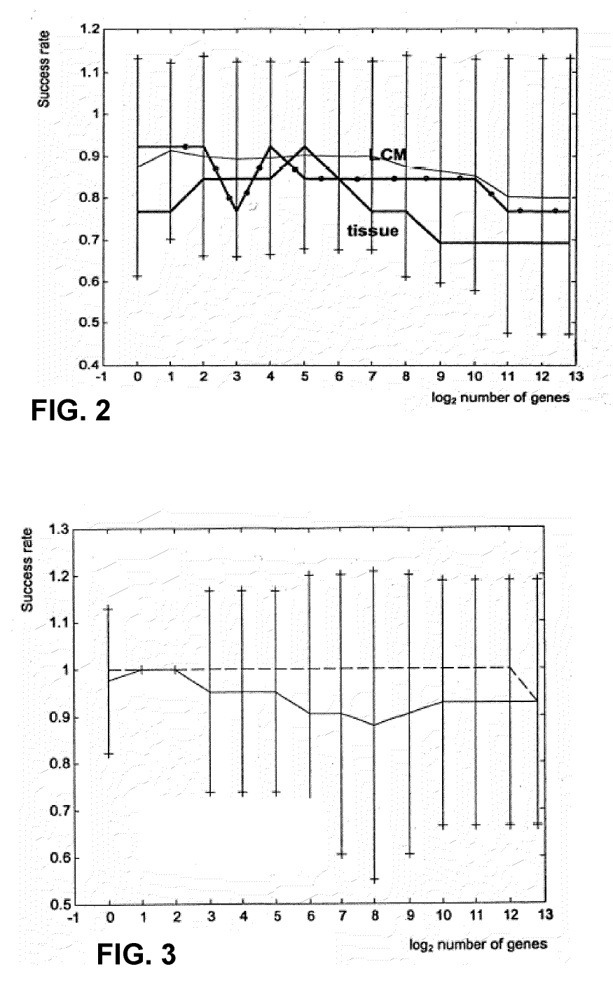

The results are shown in FIG. 2. The large error bars are due to the small size. However, there is an indication that LCM samples are better than unfiltered tissue samples. It is also interesting to note that the average curve corresponding to random splits of the data is above both curves. This is not surprising since the data in subset 1 and subset 2 are differently distributed. When making a random split rather than segregating samples, both LCM and unfiltered tissues are represented in the training and the test set and performance on the test set are better on average.

The same methods were applied to determine whether microarrays with gene expression data rejected by the Affymetrix quality criterion contained useful information by focusing on the problem of separating BPH tissue vs. G4 tissue with a total of 42 arrays (18 BPH and 24 G4).

The Affymetrix criterion identified 17 good quality arrays, 8 BPH and 9 G4.

Two subsets were formed:
Subset 1=“good” samples, 8 BPH+9 G4
Subset 2=“mediocre” samples, 10 BPH+15 G4

For comparison, all of the samples were lumped together and 10 random subset 1 containing 8 BPH+9 G4 of any quality were selected. The remaining samples were used as subset 2 allowing an average curve to be obtained. Additionally the subsets were inverted with training on the “mediocre” examples and testing on the “good” examples.

When the mediocre samples are trained, perfect accuracy on the good samples is obtained, whereas training on the good examples and testing on the mediocre yield substantially worse results.

All the BPH and G4 samples were divided into LCM and unfiltered tissue subsets to repeat similar experiments as in the previous Section:
Subset1=LCM samples (5 BPH+6 LCM)
Subset2=unfiltered tissue samples (13 BPH+18 LCM)

There, in spite of the difference in sample size, training on LCM data yields better results. In spite of the large error bars, this is an indication that the LCM data preparation method might be of help in improving sample quality.

BPH vs. G4

The Affymetrix data quality criterion were irrelevant for the purpose of determining the predictive value of particular genes and while the LCM samples seemed marginally better than the unfiltered samples, it was not possible to determine a statistical significance. Therefore, all samples were grouped together and the separation BPH vs. G4 with all 42 samples (18 BPH and 24 G4) was preformed.

To evaluate performance and compare Golub's method with SVMs, the leave-one-out method was used. The fraction of successfully classified left-out examples gives an estimate of the success rate of the various classifiers.

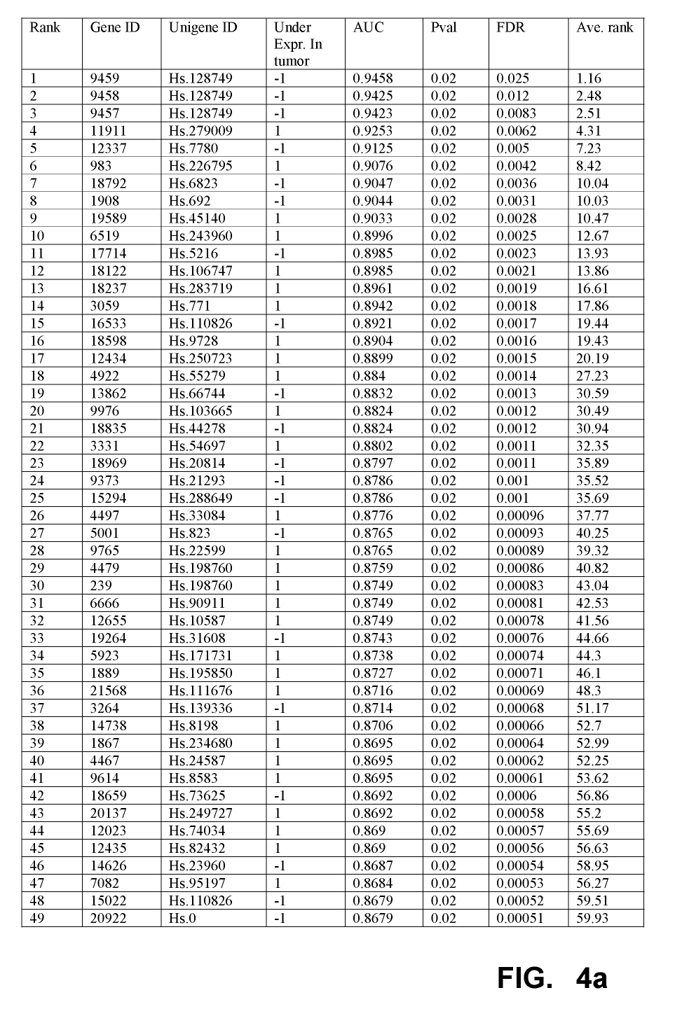

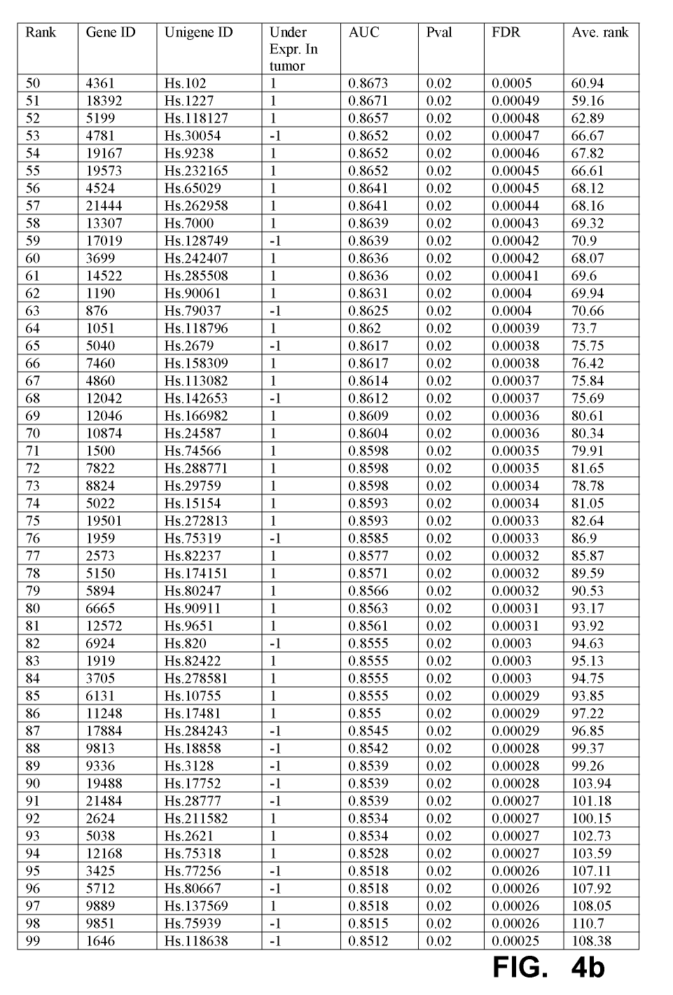

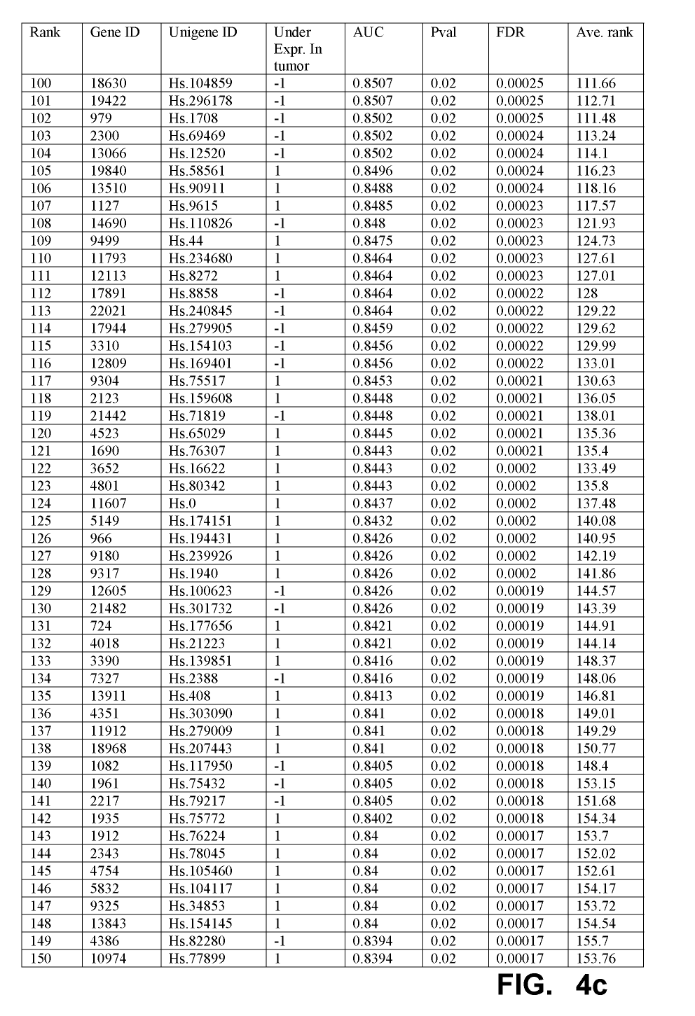

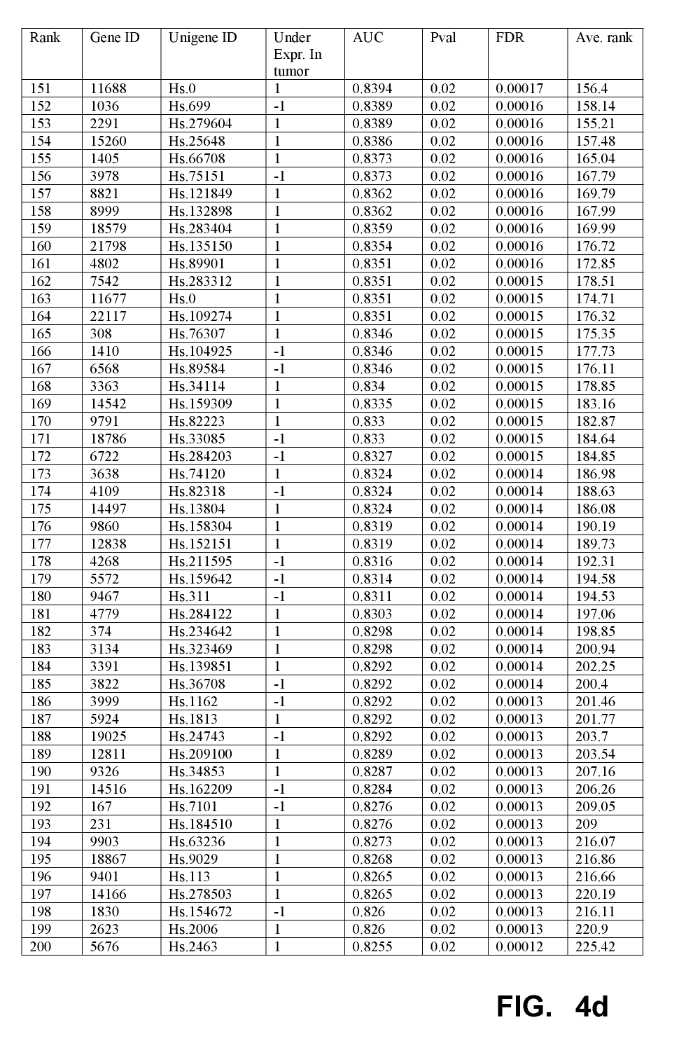

In this procedure, the gene selection process was run 41 times to obtain subsets of genes of various sizes for all 41 gene rankings. One classifier was then trained on the corresponding 40 genes for every subset of genes. This leave-one-out method differs from the “naive” leave-one-out that consists of running the gene selection only once on all 41 examples and then training 41 classifiers on every subset of genes. The naive method gives overly optimistic results because all the examples are used in the gene selection process, which is like “training on the test set”. The increased accuracy of the first method is illustrated in FIG. 3. The method used in the figure is RFE-SVM and the classifier used is an SVM. All SVMs are linear with soft margin parameters C=100 and t=10¹⁴. The dashed line represents the “naive” leave-one-out (LOO), which consists in running the gene selection once and performing loo for classifiers using subsets of genes thus derived, with different sizes. The solid line represents the more computationally expensive “true” LOO, which consists in running the gene selection 41 times, for every left out example. The left out example is classified with a classifier trained on the corresponding 40 examples for every selection of genes. If f is the success rate obtained (a point on the curve), the standard deviation is computed as sqrt(f(1-f)).

### Example 2

### Analyzing Small Data Sets with Multiple Features

Small data sets with large numbers of features present several problems. In order to address ways of avoiding data overfitting and to assess the significance in performance of multivariate and univariate methods, the samples from Example 1 that were classified by Affymetrix as high quality samples were further analyzed. The samples included 8 BPH and 9 G4 tissues. Each microarray recorded 7129 gene expression values. About ⅔ of the samples in the BPH/G4 subset were considered of inadequate quality for use with standard non-SVM methods.

Simulations resulting from multiple splits of the data set of 17 examples (8 BPH and 9 G4) into a training set and a test set were run. The size of the training set is varied. For each training set drawn, the remaining data are used for testing. For number of training examples greater than 4 and less than 16, 20 training sets were selected at random. For 16 training examples, the leave-one-out method was used, in that all the possible training sets obtained by removing 1 example at a time (17 possible choices) were created. The test set is then of size 1. Note that the test set is never used as part of the feature selection process, even in the case of the leave-one-out method.

For 4 examples, all possible training sets containing 2 examples of each class (2 BPH and 2 G4), were created and 20 of them were selected at random. For SVM methods, the initial training set size is 2 examples, one of each class (1 BPH and 1 G4). The examples of each class are drawn at random. The performance of the LDA methods cannot be computed with only 2 examples, because at least 4 examples (2 of each class) are required to compute intraclass standard deviations. The number of training examples is incremented by steps of 2.

The top ranked genes are presented in Tables 5-8. Having determined that the SVM method provided the most compact set of features to achieve 0 leave-one-out error and that the SF-SVM method is the best and most robust method for small numbers of training examples, the top genes found by these methods were researched in the literature. Most of the genes have a connection to cancer or more specifically to prostate cancer.

Table 5 shows the top ranked genes for SF LDA using 17 best BPH/G4.

TABLE 5

| Rank | GAN | EXP | Description |
|---|---|---|---|
| 10 | X83416 | −1 | H. sapiens PrP gene |
| 9 | U50360 | −1 | Human calcium calmodulin-dependent |
| | | | protein kinase II gamma mRNA |
| 8 | U35735 | −1 | Human RACH1 (RACH1) mRNA |
| 7 | M57399 | −1 | Human nerve growth factor |
| | | | (HBNF-1) mRNA |
| 6 | M55531 | −1 | Human glucose transport-like 5 |
| | | | (GLUT5) mRNA |
| 5 | U48959 | −1 | Human myosin light chain kinase |
| | | | (MLCK) mRNA |
| 4 | Y00097 | −1 | Human mRNA for protein p68 |
| 3 | D10667 | −1 | Human mRNA for smooth muscle |
| | | | myosin heavy chain |
| 2 | L09604 | −1 | Homo sapiens differentiation- |
| | | | dependent A4 protein MRNA |
| 1 | HG1612-HT1612 | 1 | McMarcks |

where GAN=Gene Acession Number; EXP=Expression (−1=underexpressed in cancer (G4) tissues; +1=overexpressed in cancer tissues).

Table 6 lists the top ranked genes obtained for LDA using 17 best BPH/G4.

TABLE 6

| Rank | GAN | EXP | Description |
|---|---|---|---|
| 10 | J03592 | 1 | Human ADP/ATP translocase mRNA |
| 9 | U40380 | 1 | Human presenilin I-374 (AD3-212) mRNA |
| 8 | D31716 | −1 | Human mRNA for GC box bindig protein |
| 7 | L24203 | −1 | Homo sapiens ataxia-telangiectasia group D |
| 6 | J00124 | −1 | Homo sapiens 50 kDa type I epidermal keratin |
| | | | gene |
| 5 | D10667 | −1 | Human mRNA for smooth muscle myosin |
| | | | heavy chain |
| 4 | J03241 | −1 | Human transforming growth factor-beta 3 |
| | | | (TGF-beta3) MRNA |
| 3 | 017760 | −1 | Human laminin S B3 chain (LAMB3) gene |
| 2 | X76717 | −1 | H. sapiens MT-11 mRNA |
| 1 | X83416 | −1 1 | H. sapiens PrP gene |

Table 7 lists the top ranked genes obtained for SF SVM using 17 best BPH/G4.

TABLE 7

| Rank | GAN | EXP | Description |
|---|---|---|---|
| 10 | X07732 | 1 | Human hepatoma mRNA for serine protease |
| | | | hepsin |
| 9 | J03241 | −1 | Human transforming growth factor-beta 3 |
| | | | (TGF-beta3) MRNA |
| 8 | X83416 | −1 | H. sapiens PrP gene |
| 7 | X14885 | −1 | H. sapiens gene for transforming growth |
| | | | factor-beta 3 (TGF-beta 3) exon 1 |
| | | | (and joined CDS) |
| 6 | U32114 | −1 | Human caveolin-2 mRNA |
| 5 | M16938 | 1 | Human homeo-box c8 protein |
| 4 | L09604 | −1 | H. sapiens differentiation-dependent A4 |
| | | | protein MRNA |
| 3 | Y00097 | −1 | Human mRNA for protein p68 |
| 2 | D88422 | −1 | Human DNA for cystatin A |
| 1 | U35735 | −1 | Human RACH1 (RACH1) mRNA |

Table 8 provides the top ranked genes for SVM using 17 best BPH/G4.

TABLE 8

| Rank | GAN | EXP | Description |
|---|---|---|---|
| 10 | X76717 | −1 | H. sapiens MT-11 mRNA |
| 9 | U32114 | −1 | Human caveolin-2 mRNA |
| 8 | X85137 | 1 | H. sapiens mRNA for kinesin-related protein |
| 7 | D83018 | −1 | Human mRNA for nel-related protein 2 |
| 6 | D10667 | −1 | Human mRNA for smooth muscle myosin |
| | | | heavy chain |
| 5 | M16938 | 1 | Human homeo box c8 protein |
| 4 | L09604 | −1 | Homo sapiens differentiation-dependent A4 |
| | | | protein mRNA |
| 3 | HG1612 | 1 | McMarcks |
| 2 | M10943 | −1 | Human metaIlothionein-If gene (hMT-If) |
| 1 | X83416 | −1 | H. sapiens PrP gene |

Using the “true” leave-one-out method (including gene selection and classification), the experiments indicate that 2 genes should suffice to achieve 100% prediction accuracy. The two top genes were therefore more particularly researched in the literature. The results are summarized in Table 10. It is interesting to note that the two genes selected appear frequently in the top 10 lists of Tables 5-8 obtained by training only on the 17 best genes.

Table 9 is a listing of the ten top ranked genes for SVM using all 42 BPH/G4.

TABLE 9

| Rank | GAN | EXP | Description |
|---|---|---|---|
| 10 | X87613 | −1 | H. sapiens mRNA for skeletal muscle abundant |
| 9 | X58072 | −1 | Human hGATA3 mRNA for trans-acting |
| | | | T-cell specific |
| 8 | M33653 | −1 | Human alpha-2 type IV collagen (COL4A2) |
| 7 | S76473 | 1 | trkB [human brain mRNA] |
| 6 | X14885 | −1 | H. sapiens gene for transforming growth |
| | | | factor-beta 3 |
| 5 | S83366 | −1 | region centromeric to t(12; 17) brakepoint |
| 4 | X15306 | −1 | H. sapiens NF-H gene |
| 3 | M30894 | 1 | Human T-cell receptor Ti rearranged |
| | | | gamma-chain |
| 2 | M16938 | 1 | Human homeo box c8 protein |
| 1 | U35735 | −1 | Human RACH1 (RACH1) mRNA |

Table 10 provides the findings for the top 2 genes found by SVM using all 42 BPH/G4. Taken together, the expression of these two genes is indicative of the severity of the disease.

TABLE 10

| GAN | Synonyms | Possible function/link to prostate cancer |
|---|---|---|
| M16938 | HOXC8 | Hox genes encode transcriptional regulatory proteins that |
| | | are largely responsible for establishing the body plan of all |
| | | metazoan organisms. There are hundreds of papers in |
| | | PubMed reporting the role of HOX genes in various |
| | | cancers. HOXC5 and HOXC8 expression are selectively |
| | | turned on in human cervical cancer cells compared to |
| | | normal keratinocytes. Another homeobox gene (GBX2) |
| | | may participate in metastatic progression in prostatic |
| | | cancer. Another HOX protein (hoxb-13) was identified as |
| | | an androgen-independent gene expressed in adult mouse |
| | | prostate epithelial cells. The authors indicate that this |
| | | provides a new potential target for developing therapeutics to treat |
| | | advanced prostate cancer |
| U35735 | Jk | Overexpression of RACH2 in human tissue culture cells |
| | Kidd | induces apoptosis. RACH1 is downregulated in breast |
| | RACH1 | cancer cell line MCF-7. RACH2 complements the RAD1 |
| | RACH2 | protein. RAM is implicated in several cancers. |
| | SLC14A1 | Significant positive lod scores of 3.19 for linkage of the Jk |
| | UT1 | (Kidd blood group) with cancer family syndrome (CFS) |
| | UTE | were obtained. CFS gene(s) may possibly be located on |
| | | chromosome 2, where Jk is located. |

Table 11 shows the severity of the disease as indicated by the top 2 ranking genes selected by SVMs using all 42 BPH and G4 tissues.

TABLE 11

| | | HOXC8 |
|---|---|---|
| | HOXC8 Underexpressed | Overexpressed |
| RACH1Overexpressed | Benign | N/A |
| RACH1 Underexpressed | Grade 3 | Grade 4 |

### Example 3

### Prostate Cancer Study on Affymetrix Gene Expression Data (09-2004)

A set of Affymetrix microarray GeneChip® experiments from prostate tissues were obtained from Dr. Thomas A. Stamey at Stanford University. The data from samples obtained for the prostate cancer study are summarized in Table 12 (which represents the same data as in Table 3 but organized differently.) Preliminary investigation of the data included determining the potential need for normalizations. Classification experiments were run with a linear SVM on the separation of Grade 4 tissues vs. BPH tissues. In a 32×3-fold experiment, an 8% error rate could be achieved with a selection of 100 genes using the multiplicative updates technique (similar to RFE-SVM). Performances without feature selection are slightly worse but comparable. The gene most often selected by forward selection was independently chosen in the top list of an independent published study, which provided an encouraging validation of the quality of the data.

TABLE 12

| Prostate zone | Histological classification | No. of samples |
|---|---|---|
| Central (CZ) | Normal (NL) | 9 |
| | Dysplasia (Dys) | 4 |
| | Grade 4 cancer (G4) | 1 |
| Peripheral (PZ) | Normal (NL) | 13 |
| | Dysplasia (Dys) | 13 |
| | Grade 3 cancer (G3) | 11 |
| | Grade 4 cancer (G4) | 18 |
| Transition (TZ) | Benign Prostate Hyperplasia (BPH) | 10 |
| | Grade 4 cancer (G4) | 8 |
| Total | | 87 |

As controls, normal tissues and two types of abnormal tissues are used in the study: BPH and Dysplasia.

To verify the data integrity, the genes were sorted according to intensity. For each gene, the minimum intensity across all experiments was taken. The top 50 most intense values were taken. Heat maps of the data matrix were made by sorting the lines (experiments) according to zone, grade, and time processed. No correlation was found with zone or grade, however, there was a significant correlation with the time the sample was processed. Hence, the arrays are poorly normalized.

In other ranges of intensity, this artifact is not seen. Various normalization techniques were tried, but no significant improvements were obtained. It has been observed by several authors that microarray data are log-normal distributed. A qqplot of all the log of the values in the data matrix confirms that the data are approximately log-normal distributed. Nevertheless, in preliminary classification experiments, there was not a significant advantage of taking the log.

Tests were run to classify BPH vs. G4 samples. There were 10 BPH samples and 27 G4 samples. 32×3 fold experiments were performed in which the data was split into 3 subsets 32 times. Two of the subsets were used for training while the third was used for testing. The results were averaged. A feature selection was performed for each of the 32×3 data splits; the features were not selected on the entire dataset.

A linear SVM was used for classification, with ridge parameter 0.1, adjusted for each class to balance the number of samples per class. Three feature selection methods were used: (1) multiplicative updates down to 100 genes (MU100); (2) forward selection with approximate gene orthogonalisation up to 2 genes (FS2); and (3) no gene selection (NO).

The data was either raw or after taking the log(LOG). The genes were always standardized (STD: the mean over all samples is subtracted and the result is divided by the standard deviation; mean and stdev are computed on training data only, the same coefficients are applied to test data).

The results for the performances for the BPH vs. G4 separation are shown in Table 13 below, with the standard errors are shown in parentheses. “Error rate” is the average number of misclassification errors; “Balanced errate” is the average of the error rate of the positive class and the error rate of the negative class; “AUC” is the area under the ROC (receiver operating characteristic) curves that plots the sensitivity (error rate of the positive class, G4) as a function of the specificity (error rate of the negative class, BPH).

It was noted that the SVM performs quite well without feature selection, and MU 100 performs similarly, but slightly better. The number of features was not adjusted—100 was chosen arbitrarily.

TABLE 13

| | | | Balanced | |
|---|---|---|---|---|
| Preprocessing | Feat. Select. | Error rate | errate | AUC |
| Log + STD | MU 100 | 8.09 (0.66) | 11.68 (1.09) | 98.93 (0.2) |
| Log + STD | FS 2 | 13.1 (1.1) | 15.9 (1.3) | 92.02 (1.15) |
| Log + STD | No selection | 8.49 (0.71) | 12.37 (1.13) | 97.92 (0.33) |
| STD | No selection | 8.57 (0.72) | 12.36 (1.14) | 97.74 (0.35) |

In Table 13, the good AUC and the difference between the error rate and the balanced error rate show that the bias of the classifier must be optimized to obtained a desired tradeoff between sensitivity and specificity.

Two features are not enough to match the best performances, but do quite well already.

It was determined which features were selected most often with the FS 2 method. The first gene (3480) was selected 56 times, while the second best one (5783) was selected only 7 times. The first one is believed to be relevant to cancer, while the second one has probably been selected for normalization purposes. It is interesting that the first gene (Hs.79389) is among the top three genes selected in another independent study (Febbo-Sellers, 2003).

The details of the two genes are as follows:
Gene 3480: gb:NM_{—}006159.1/DEF=Homo sapiens nel (chicken)-like 2 (NELL2), mRNA./FEA=mRNA/GEN=NELL2/PROD=nel (chicken)-like2/DB_XREF=gi:5453765/UG=Hs.79389 nel (chicken)-like 2/FL=gb:D83018.1 gb:NM_{—}006159.1
Gene 5783: gb:NM_{—}018843.1/DEF=Homo sapiens mitochondrial carrier family protein(LOC55972), mRNA./FEA=mRNA/GEN=LOC55972 /PROD=mitochondrial carrier family protein /DB_XREF=gi: 10047121 /UG=Hs.172294 mitochondrial carrier family protein /FL=gb:NM_{—}018843.1 gb:AF125531.1.

### Example 4

### Prostate Cancer Study from Affymetrix Gene Expression Data (10-2004)

This example is a continuation of the analysis of Example 3 above on the Stamey prostate cancer microarray data. PSA has long been used as a biomarker of prostate cancer in serum, but is no longer useful. Other markers have been studied in immunohistochemical staining of tissues, including p27, Bcl-2, E-catherin and P53. However, to date, no marker has gained acceptance for use in routine clinical practice.

The gene rankings obtained correlate with those of the Febbo paper, confirming that the top ranking genes found from the Stamey data have a significant intersection with the genes found in the Febbo study. In the top 1000 genes, about 10% are Febbo genes. In comparison, a random ordering would be expected to have less than 1% are Febbo genes.

BPH is not by itself an adequate control. When selecting genes according to how well they separate grade 4 cancer tissues (G4) from BPH, one can find genes that group all non-BPH tissues with the G4 tissues (including normal, dysplasia and grade 3 tissues). However, when BPH is excluded from the training set, genes can be found that correlate well with disease severity. According to those genes, BPH groups with the low severity diseases, leading to a conclusion that BPH has its own molecular characteristics and that normal adjacent tissues should be used as controls.

TZG4 is less malignant than PZG4. It is known that TZ cancer has a better prognosis than PZ cancer. The present analysis provides molecular confirmation that TZG4 is less malignant than PZG4. Further, TZG4 samples group with the less malignant samples (grade 3, dysplasia, normal, or BPH) than with PZG4. This differentiated grouping is emphasized in genes correlating with disease progression (normal<dysplasia<g3<g4) and selected to provide good separation of TZG4 from PZG4 (without using an ordering for TZG4 and PZG4 in the gene selection criterion).

Ranking criteria implementing prior knowledge about disease malignancy are more reliable. Ranking criteria validity was assessed both with p values and with classification performance. The criterion that works best implements a tissue ordering normal<dysplasia<G3<G4 and seeks a good separation TZG4 from PZG4. The second best criterion implements the ordering normal<dysplasia<G3<TZG4<PZG4.

Comparing with other studies may help reducing the risk of overfitting. A subset of 7 genes was selected that ranked high in the present study and that of Febbo et al. 2004. Such genes yield good separating power for G4 vs. other tissues. The training set excludes BPH samples and is used both to select genes and train a ridge regression classifier. The test set includes 10 BPH and 10 G4 samples (½ from the TZ and ½ from the PZ). Success was evaluated with the area under the ROC curve (“AUC”)(sensitivity vs. specificity) on test examples. AUCs between 0.96 and 1 are obtained, depending on the number of genes. Two genes are of special interest (GSTP1 and PTGDS) because they are found in semen and could be potential biomarkers that do not require the use of biopsied tissue.

The choice of the control may influence the findings (normal tissue or BPH). as may the zones from which the tissues originate. The first test sought to separate Grade 4 from BPH. Two interesting genes were identified by forward selection as gene 3480 (NELL2) and gene 5783(LOC55972). As explained in Example 3, gene 3480 is the informative gene, and it is believed that gene 5783 helps correct local on-chip variations. Gene 3480, which has Unigene cluster id. Hs.79389, is a Nel-related protein, which has been found at high levels in normal tissue by Febbo et al.

All G4 tissues seem intermixed regardless of zone. The other tissues are not used for gene selection and they all fall on the side of G4. Therefore, the genes found characterize BPH, not G4 cancer, such that it is not sufficient to use tissues of G4 and BPH to find useful genes to characterize G4 cancer.

For comparison, two filter methods were used: the Fisher criterion and the shrunken centroid criterion (Tibshirani et al, 2002). Both methods found gene 3480 to be highly informative (first or second ranking). The second best gene is 5309, which has Unigene cluster ID Hs. 100431 and is described as small inducible cytokine B subfamily (Cys-X-Cys motif). This gene is highly correlated to the first one.

The Fisher criterion is implemented by the following routine:
A vector x containing the values of a given feature for all patt_num samples
cl_num classes, k=1, 2, . . . cl_num, grouping the values of x
mu_val(k) is the mean of the x values for class k
var_val(k) is the variance of the x values for class k
patt_{—}4 per class(k) is the number of elements of class k
Unbiased_within_var is the unbiased pooled within class variance, i.e., we make a weighted average of var_val(k) with coefficients patt_per class(k)/(patt_num—cl_num)
Unbiased_between_var=var(mu_val); % Divides by cl_num−1 then
Fisher_crit=Unbiased_between_var /Unbiased_within_var

Although the shrunken centroid criterion is somewhat more complicated that the Fisher criterion, it is quite similar. In both cases, the pooled within class variance is used to normalize the criterion. The main difference is that instead of ranking according to the between class variance (that is, the average deviation of the class centroids to the overall centroid), the shrunken centroid criterion uses the maximum deviation of any class centroid to the global centroid. In doing so, the criterion seeks features that well separate at least one class, instead of features that well separate all classes (on average).

The other small other differences are:
A fudge factor is added to Unbiased_within_std=sqrt(Unbiased_within_var) to prevent divisions by very small values. The fudge factor is computed as:
fudge=mean(Unbiased_within_std); the mean being taken over all the features.
Each class is weighted according to its number of elements cl_elem(k). The deviation for each class is weighted by 1/sqrt(1/cl_elem(k)+1/patt_num). Similar corrections could be applied to the Fisher criterion.

The two criteria are compared using pvalues. The Fisher criterion produces fewer false positive in the top ranked features. It is more robust, however, it also produces more redundant features. It does not find discriminant features for the classes that are least abundant or hardest to separate.

Also for comparison, the criterion of Golub et al., also known as signal to noise ratio, was used. This criterion is used in the Febbo paper to separate tumor vs. normal tissues. On this data that the Golub criterion was verified to yield a similar ranking as the Pearson correlation coefficient. For simplicity, only the Golub criterion results are reported. To mimic the situation, three binary separations were run: (G3+4 vs. all other tissues), (G4 vs. all other tissues), and (G4 vs. BPH). As expected, the first gene selected for the G4 vs. BPH is 3480, but it does not rank high in the G3+4 vs. all other and G4 vs. all other.

Compared to a random ranking, the genes selected using the various criteria applied are enriched in Febbo genes, which cross-validates the two study. For the multiclass criteria, the shrunken centroid method provides genes that are more different from the Febbo genes than the Fisher criterion. For the two-class separations, the tumor vs normal (G3+4 vs others) and the G4 vs. BPH provide similar Febbo enrichment while the G4 vs. all others gives gene sets that depart more from the Febbo genes. Finally, it is worth noting that the initial enrichment up to 1000 genes is of about 10% of Febbo genes in the gene set. After that, the enrichment decreases. This may be due to the fact that the genes are identified by their Unigene Ids and more than one probe is attributed to the same Id. In any case, the enrichment is very significant compared to the random ranking.

A number of probes do not have Unigene numbers. Of 22,283 lines in the Affymetrix data, 615 do not have Unigene numbers and there are only 14,640 unique Unigene numbers. In 10,130 cases, a unique matrix entry corresponds to a particular Unigene ID. However, 2,868 Unigene IDs are represented by 2 lines, 1,080 by 3 lines, and 563 by more than 3 lines. One Unigene ID covers 13 lines of data. For example, Unigene ID Hs.20019, identifies variants of Homo sapiens hemochromatosis (HFE) corresponding to GenBank accession numbers: AF115265.1, NM_{—}000410.1, AF144240.1, AF150664.1, AF149804.1, AF144244.1, AF115264.1, AF144242.1, AF144243.1, AF144241.1, AF079408.1, AF079409.1, and (consensus) BG402-460.

The Unigene IDs of the paper of Febbo et al. (2003) were compared using the U95AV2 Affymetrix array and the IDs found in the U133A array under study. The Febbo paper reported 47 unique Unigene IDs for tumor high genes, 45 of which are IDs also found in the U133A array. Of the 49 unique Unigene IDs for normal high genes, 42 are also found in the U133A array. Overall, it is possible to see cross-correlations between the findings. There is a total of 96 Febbo genes that correspond to 173 lines (some genes being repeated) in the current matrix.

Based on the current results, one can either conclude that the “normal” tissues that are not BPH and drawn near the cancer tissues are on their way to cancer, or that BPH has a unique molecular signature that, although it may be considered “normal”, makes it unfit as a control. A test set was created using 10 BPH samples and 10 grade 4 samples. Naturally, all BPH are in the TZ. The grade 4 are ½ in the TZ and ½ in the PZ.

Gene selection experiments were performed using the following filter methods:

(1)—Pearson's correlation coefficient to correlate with disease severity, where disease severity is coded as normal=1, dysplasia=2, grade3=3, grade4=4.

(2)—Fisher's criterion to separate the 4 classes (normal, dysplasia, grade3, grade4) with no consideration of disease severity.

(3)—Fisher's criterion to separate the 3 classes (PZ, CZ, TZ)

(4)—Relative Fisher criterion by computing the ratio of the between class variances of the disease severity and the zones, in an attempt to de-emphasize the zone factor.

(5)—Fisher's criterion to separate 8 classes corresponding to all the combinations of zones and disease severity found in the training data.

(6)—Using the combination of 2 rankings: the ranking of (1) and a ranking by zone for the grade 4 samples only. The idea is to identify genes that separate TZ from PZ cancers that have a different prognosis.

For each experiment, scatter plots were analyzed for the two best selected genes, the heat map of the 50 top ranked genes was reviewed, and p values were compared. The conclusions are as follows:

The Pearson correlation coefficient tracking disease severity (Experiment (1)) gives a similar ranking to the Fisher criterion, which discriminates between disease classes without ranking according to severity. However, the Pearson criterion has slightly better p values and, therefore, may give fewer false positives. The two best genes found by the Pearson criterion are gene 6519, ranked 6^{th} by the Fisher criterion, and gene 9457, ranked 1^{st} by the Fisher criterion. The test set examples are nicely separated, except for one outlier.

The zonal separation experiments were not conclusive because there are only 3 TZ examples in the training set and no example of CZ in the test set. Experiment (3) revealed a good separation of PZ and CZ on training data. TZ was not very well separated. Experiments (4) and (5) did not show very significant groupings. Experiment (6) found two genes that show both disease progression and that TZ G4 is grouped with “less severe diseases” than PZ G4, although that constraint was not enforced. To confirm the latter finding, the distance for the centroids of PZG4 and TZG4 were compared to control samples. Using the test set only (controls are BPH), 63% of all the genes show that TZG4 is closer to the control than PZG4. That number increases to 70% if the top 100 genes of experiment (6) are considered. To further confirm, experiment (6) was repeated with the entire dataset (without splitting between training and test). TZG4 is closer to normal than PZG4 for most top ranked genes. In the first 15 selected genes, 100% have TZG4 closer to normal than PZG4. This finding is significant because TZG4 has better prognosis than PZG4.

Classification experiments were performed to assess whether the appropriate features had been selected using the following setting:

The data were split into a training set and a test set. The test set consists of 20 samples: 10 BPH, 5 TZG4 and 5 PZG4. The training set contains the rest of the samples from the data set, a total of 67 samples (9 CZNL, 4 CZDYS, 1 CZG4, 13 PZNL, 13 PZDYS, 11PZG3, 13 PZG4, 3 TZG4). The training set does not contain any BPH.

Feature selection was performed on training data only. Classification was performed using linear ridge regression. The ridge value was adjusted with the leave-one-out error estimated using training data only. The performance criterion was the area under the ROC curve (AUC), where the ROC curve is a plot of the sensitivity as a function of the specificity. The AUC measures how well methods monitor the tradeoff sensitivity/specificity without imposing a particular threshold.

P values are obtained using a randomization method proposed by Tibshirani et al. Random “probes” that have a distribution similar to real features (gene) are obtained by randomizing the columns of the data matrix, with samples in lines and genes in columns. The probes are ranked in a similar manner as the real features using the same ranking criterion. For each feature having a given score s, where a larger score is better, a p value is obtained by counting the fraction of probes having a score larger than s. The larger the number of probes, the more accurate the p value.

For most ranking methods, and for forward selection criteria using probes to compute p values does not affect the ranking. For example, one can rank the probes and the features separately for the Fisher and Pearson criteria.

P values measure the probability that a randomly generated probe imitating a real gene, but carrying no information, gets a score larger or equal to s. Considering a single gene, if it has a score of s, the p value test can be used to test whether to reject the hypothesis that it is a random meaningless gene by setting a threshold on the p value, e.g., 0.0. The problem is that there are many genes of interest (in the present study, N=22,283.) Therefore, it becomes probable that at least one of the genes having a score larger than s will be meaningless. Considering many genes simultaneously is like doing multiple testing in statistics. If all tests are independent, a simple correction known as the Bonferroni correction can be performed by multiplying the p values by N. This correction is conservative when the test are not independent.

From p values, one can compute a “false discovery rate” as FDR(s)=pvalue(s)*N/r, where r is the rank of the gene with score s, pvalue(s) is the associated p value, N is the total number of genes, and pvalue(s)*N is the estimated number of meaningless genes having a score larger than s. FDR estimates the ratio of the number of falsely significant genes over the number of genes call significant.

Of the classification experiments described above, the method that performed best was the one that used the combined criteria of the different classification experiments. In general, imposing meaningful constraints derived from prior knowledge seems to improve the criteria. In particular, simply applying the Fisher criterion to the G4 vs. all-the-rest separation (G4vsAll) yields good separation of the training examples, but poorer generalization than the more constrained criteria. Using a number of random probes equal to the number of genes, the G4vsAll identifies 170 genes before the first random probe, multiclass Fisher obtains 105 and the Pearson criterion measuring disease progression gets 377. The combined criteria identifies only 8 genes, which may be attributed to the different way in which values are computed. With respect to the number of Febbo genes found in the top ranking genes, G4 vs All has 20, multiclass Fisher 19, Pearson 19, and the combined criteria 8. The combined criteria provide a characterization of zone differentiation. On the other hand, the top 100 ranking genes found both by Febbo and by criteria G4 vs All, Fisher or Pearson have a high chance of having some relevance to prostate cancer. These genes are listed in Table 14.

TABLE 14

| Order | | | | G4 vs | | |
|---|---|---|---|---|---|---|
| Num | Unigene ID | Fisher | Pearson | ALL | AUC | Description |
| 12337 | Hs.7780 | 11 | 6 | 54 | 0.96 | cDNA DKFZp56A072 |
| 893 | Hs.226795 | 17 | 7 | 74 | 0.99 | Glutathione S-transferase pi (GSTP1) |
| 5001 | Hs.823 | 41 | 52 | 72 | 0.96 | Hepsin (transmembrance protease, |
| | | | | | | serine 1) (HPN) |
| 1908 | Hs.692 | 62 | 34 | 111 | 0.96 | Tumor-associated calcium signal |
| | | | | | | transducer 1 (TACSTD1) |
| 5676 | Hs.2463 | 85 | 317 | 151 | 1 | Angiopoietin 1 (ANGPT1) |
| 12113 | Hs.8272 | 181 | 93 | 391 | 1 | Prostaglandin D2 synthase (21 kD, |
| | | | | | | brain) (PTGDS) |
| 12572 | Hs.9651 | 96 | 131 | 1346 | 0.99 | RAS related viral oncogene homolog |
| | | | | | | (RRAS) |

Table 14 shows genes found in the top 100 as determined by the three criteria, Fisher, Pearson and G4vsALL, that were also reported in the Febbo paper. In the table, Order num is the order in the data matrix. The numbers in the criteria columns indicate the rank. The genes are ranked according to the sum of the ranks of the 3 criteria. Classifiers were trained with increasing subset sizes showing that a test AUC of 1 is reached with 5 genes.

The published literature was checked for the genes listed in Table 14. Third ranked Hepsin has been reported in several papers on prostate cancer: Chen et al. (2003) and Febbo et al. (2003) and is picked up by all criteria. Polymorphisms of second ranked GSTP1 (also picked by all criteria) are connected to prostate cancer risk (Beer et al, 2002). The fact that GSTP1 is found in semen (Lee (1978)) makes it a potentially interesting marker for non-invasive screening and monitoring. The clone DKFZp564A072, ranked first, is cited is several gene expression studies.

Fourth ranked Gene TACSTD1 was also previously described as more-highly expressed in prostate adenocarcinoma (see Lapointe et al, 2004 and references therein). Angiopoietin (ranked fifth) is involved in angiogenesis and known to help the blood irrigation of tumors in cancers and, in particular, prostate cancer (see e.g. Cane, 2003). Prostaglandin D2 synthase (ranked sixth) has been reported to be linked to prostate cancer in some gene expression analysis papers, but more interestingly, prostaglandin D synthase is found in semen (Tokugawa, 1998), making it another biomarker candidate for non-invasive screening and monitoring. Seventh ranked RRAS is an oncogene, so it makes sense to find it in cancer, however, its role in prostate cancer has not been documented.

A combined criterion was constructed for selecting genes according to disease severity NL<DYS<G3<G4 and simultaneously tries to differentiate TZG4 from PZG4 without ordering them. This following procedure was used:
Build an ordering using the Pearson criterion with encoded target vector having values NL=1, DYS=2, G3=3, G4=4 (best genes come last.)
Build an ordering using the Fisher criterion to separate TZG4 from PZG$ (best genes come last.)
Obtain a combined criterion by adding for each gene its ranks obtained with the first and second criterion.

Sort according to the combined criterion (in descending order, best first).

P values can be obtained for the combined criterion as follows:
Unsorted score vectors for real features (genes) and probes are concatenated for both criteria (Pearson and Fisher).
Genes and probes are sorted together for both criteria, in ascending order (best last).

The combined criterion is obtained by summing the ranks, as described above.
For each feature having a given combined criterion value s (larger values being better), a p value is obtained by counting the fraction of probes a having a combined criterion larger than s.

Note that this method for obtaining p values disturbs the ranking, so the ranking that was obtained without the probes listed in Table 15 was used.

A listing of genes obtained with the combined criterion are shown in Table 15. The ranking is performed on training data only. “Order num” designates the gene order number in the data matrix; p values are adjusted by the Bonferroni correction; “FDR” indicates the false discovery rate; “Test AUC” is the area under the ROC curve computed on the test set; and “Cancer cor” indicates over-expression in cancer tissues.

TABLE 15

| | Order | Unigene | P | | Test | Cancer | |
|---|---|---|---|---|---|---|---|
| Rank | num | ID | value | FDR | AUC | cor | Gene description |
| 1 | 3059 | Hs.771 | <0.1 | <0.01 | 0.96 | −1 | gb: NM_002863.1 /DEF = Homo sapiens |
| | | | | | | | phosphorylase, /UG = Hs.771 phosphorylase, |
| | | | | | | | glycogen; liver |
| 2 | 13862 | Hs.66744 | <0.1 | <0.01 | 0.96 | 1 | Consensus includes |
| | | | | | | | gb: X99268.1/DEF = H./FL = gb: NM_000474.1 |
| 3 | 13045 | Hs.173094 | <0.1 | <0.01 | 1 | −1 | Consensus includes gb: AI096375/FEA = EST |
| 4 | 5759 | Hs.66052 | <0.1 | <0.01 | 0.97 | −1 | gb: NM_001775.1/DEF = Homo sapiens CD38 |
| 5 | 18621 | Hs.42824 | <0.1 | <0.01 | 0.95 | −1 | gb: NM_018192.1/DEF = Homo sapiens |
| | | | | | | | hypothetical |
| 6 | 3391 | Hs.139851 | <0.1 | <0.01 | 0.94 | −1 | gb: NM_001233.1/DEF = Homo sapiens caveolin |
| 7 | 18304 | Hs.34045 | <0.1 | <0.01 | 0.95 | 1 | gb: NM_017955.1/DEF = Homo sapiens |
| | | | | | | | hypothetical |
| 8 | 14532 | Hs.37035 | <0.1 | <0.01 | 1 | 1 | Consensus includes gb: AI738662/FEA = EST |
| 9 | 3577 | Hs.285754 | 0.1 | 0.01 | 1 | −1 | Consensus includes gb: BG170541/FEA = EST |
| 10 | 9010 | Hs.180446 | 0.1 | 0.01 | 1 | 1 | gb: L38951.1/DEF = Homo sapiens importin |
| 11 | 13497 | Hs.71465 | 0.1 | 0.01 | 1 | −1 | Consensus includes gb: AA639705/FEA = EST |
| 12 | 19488 | Hs.17752 | 0.1 | 0.01 | 1 | 1 | gb: NM_015900.1/DEF = Homo sapiens phosph |
| | | | | | | | phospholipase A1alpha/FL = gb: AF035268.1 |
| 13 | 8838 | Hs.237825 | 0.1 | 0.01 | 1 | 1 | gb: AF069765.1/DEF = Homo sapiens signal |
| | | | | | | | gb: NM_006947.1 |
| 14 | 14347 | Hs.170250 | 0.1 | 0.01 | 1 | 1 | Consensus includes gb: K02403.1/DEF = Human |
| 15 | 2300 | Hs.69469 | 0.2 | 0.01 | 1 | 1 | gb: NM_006360.1/DEF = Homo sapiens dendritic |
| 16 | 10973 | Hs.77899 | 0.2 | 0.01 | 1 | −1 | gb: Z24727.1/DEF = H. sapiens tropomyosin |
| 17 | 11073 | Hs.0 | 0.2 | 0.01 | 1 | 1 | gb: Z25434.1/DEF = H. sapiens protein- |
| | | | | | | | serinethreonine |
| 18 | 22193 | Hs.165337 | 0.2 | 0.01 | 1 | −1 | Consensus includes gb: AW971415/FE |
| 19 | 12742 | Hs.237506 | 0.2 | 0.01 | 1 | −1 | Consensus includes gb: AK023253.1/DEF = |
| 20 | 21823 | Hs.9614 | 0.3 | 0.01 | 1 | 1 | Consensus includes gb: AA191576/FEA = EST |
| 21 | 13376 | Hs.246885 | 0.3 | 0.01 | 1 | −1 | Consensus includes gb: W87466/FEA = EST |
| 22 | 6182 | Hs.77899 | 0.3 | 0.01 | 1 | −1 | gb: NM_000366.1/DEF = Homo sapiens |
| | | | | | | | tropomyosin |
| 23 | 3999 | Hs.1162 | 0.4 | 0.02 | 1 | 1 | gb: NM_002118.1/DEF = Homo sapiens major II, |
| | | | | | | | DM beta/FL = gb: NM_002118.1 gb: U15085.1 |
| 24 | 1776 | Hs.168670 | 0.7 | 0.03 | 1 | −1 | gb: NM_002857.1/DEF = Homo sapiens |
| | | | | | | | peroxisomal gb: AB018541.1 |
| 25 | 4046 | Hs.82568 | 0.7 | 0.03 | 1 | −1 | gb: NM_000784.1/DEF = Homo sapiens cytochrome |
| | | | | | | | cerebrotendinous xanthomatosis), polypeptide |
| 26 | 6924 | Hs.820 | 0.8 | 0.03 | 1 | 1 | gb: NM_004503.1/DEF = Homo sapiens homeo |
| 27 | 2957 | Hs.1239 | 0.9 | 0.03 | 1 | −1 | gb: NM_001150.1/DEF = Homo sapiens |
| | | | | | | | alanyl/DB_XREF = gi: 4502094/UG = Hs.1239 |
| | | | | | | | alanyl |
| 28 | 5699 | Hs.78406 | 1.3 | 0.05 | 1 | −1 | gb: NM_003558.1/DEF = Homo sapiens |
| | | | | | | | phosphatidylinositol phosphate 5-kinase, type I, |
| | | | | | | | beta/FL = gb: NM |
| 29 | 19167 | Hs.9238 | 1.4 | 0.05 | 1 | −1 | gb: NM_024539.1/DEF = Homo sapiens |
| | | | | | | | hypothetical |
| 30 | 4012 | Hs.172851 | 1.4 | 0.05 | 1 | −1 | gb: NM_001172.2/DEF = Homo sapiens arginase, |
| | | | | | | | gb: D86724.1 gb: U75667.1 gb: U82256.1 |
| 31 | 9032 | Hs.80658 | 1.4 | 0.05 | 1 | −1 | gb: U94592.1/DEF = Human uncoupling protein |
| | | | | | | | gb: U82819.1 gb: U94592.1 |
| 32 | 15425 | Hs.20141 | 1.5 | 0.05 | 1 | 1 | Consensus includes gb: AK000970.1/DEF= |
| 33 | 14359 | Hs.155956 | 1.6 | 0.05 | 1 | −1 | Consensus includes |
| | | | | | | | gb: NM_000662.1/DEF = acetyltransferase)/FL = gb: |
| | | | | | | | NM_000662.1 |
| 34 | 6571 | Hs.89691 | 1.6 | 0.05 | 1 | 1 | gb: NM_021139.1/DEF = Homo sapiens UDP |
| | | | | | | | polypeptide B4/FL = gb: NM_021139.1 |
| | | | | | | | gb: AF064200.1 |
| 35 | 13201 | Hs.301552 | 1.8 | 0.05 | 1 | 1 | Consensus includes gb: AK000478.1/DEF= |
| 36 | 21754 | Hs.292911 | 1.8 | 0.05 | 1 | −1 | Consensus includes gb: AI378979/FEA = EST |
| 37 | 5227 | Hs.31034 | 2 | 0.05 | 1 | −1 | Consensus includes gb: AL360141.1/DEF= |
| 38 | 18969 | Hs.20814 | 2.1 | 0.06 | 1 | 1 | gb: NM_015955.1/DEF = Homo sapiens CGI |
| 39 | 17907 | Hs.24395 | 2.2 | 0.06 | 1 | 1 | gb: NM_004887.1/DEF = Homo sapiens small small |
| | | | | | | | inducible cytokine subfamily B (Cys |
| 40 | 3831 | Hs.77695 | 2.3 | 0.06 | 1 | 1 | gb: NM_014750.1/DEF = Homo sapiens KIAA0008 |
| 41 | 10519 | Hs.4975 | 2.4 | 0.06 | 0.98 | 1 | gb: D82346.1/DEF = Homo sapiens mRNA |
| 42 | 2090 | Hs.150580 | 2.4 | 0.06 | 0.97 | −1 | gb: AF083441.1/DEF = Homo sapiens SUI1 |
| 43 | 9345 | Hs.75244 | 2.6 | 0.06 | 0.97 | −1 | gb: D87461.1/DEF = Human mRNA for KIAA0271 |
| 44 | 3822 | Hs.36708 | 2.7 | 0.06 | 0.97 | 1 | gb: NM_001211.2/DEF = Homo sapiens budding |
| | | | | | | | uninhibited by benzimidazoles 1 (yeast homolog) |
| 45 | 17999 | Hs.179666 | 2.9 | 0.06 | 0.97 | −1 | gb: NM_018478.1/DEF = Homo sapiens |
| | | | | | | | uncharacterized HSMNP1/FL = gb: BC001105.1 |
| | | | | | | | gb: AF220191.1 |
| 46 | 5070 | Hs.118140 | 2.9 | 0.06 | 0.96 | 1 | gb: NM_014705.1/DEF = Homo sapiens KIAA0716 |
| 47 | 20627 | Hs.288462 | 3 | 0.06 | 0.98 | −1 | gb: NM_025087.1/DEF = Homo sapiens |
| | | | | | | | hypothetical |
| 48 | 14690 | Hs.110826 | 3 | 0.06 | 0.99 | 1 | Consensus includes gb: AK027006.1/DEF= |
| 49 | 18137 | Hs.9641 | 3 | 0.06 | 0.98 | 1 | gb: NM_015991.1/DEF = Homo sapiens |
| | | | | | | | complement component 1, q subcomponent, alpha |
| | | | | | | | polypeptide-1 |
| 50 | 9594 | Hs.182278 | 3 | 0.06 | 0.98 | −1 | gb: BC000454.1/DEF = Homo sapiens, |
| | | | | | | | cal/FL = gb: BC000454.1 |

From Table 15, the combined criteria give an AUC of 1 between 8 and 40 genes. This indicates that subsets of up to 40 genes taken in the order of the criteria have a high predictive power. However, genes individually can also be judged for their predictive power by estimating p values. P values provide the probability that a gene is a random meaningless gene. A threshold can be set on that p value, e.g. 0.05.

Using the Bonferroni correction ensures that p values are not underestimated when a large number of genes are tested. This correction penalizes p values in proportion to the number of genes tested. Using 10*N probes (N=number of genes) the number of genes that score higher than all probes are significant at the threshold 0.1. Eight such genes were found with the combined criterion, while 26 genes were found with a p value<1.

It may be useful to filter out as many genes as possible before ranking them in order to avoid an excessive penalty. When the genes were filtered with the criterion that the standard deviation should exceed twice the mean (a criterion not involving any knowledge of how useful this gene is to predict cancer). This reduced the gene set to N′=571, but there were also only 8 genes at the significance level of 0.1 and 22 genes had p value<1.

The 8 first genes found by this method are given in Table 16. Genes over-expressed in cancer are under Rank 2, 7, and 8 (underlined). The remaining genes are under-expressed.

TABLE 16

| Rank | Unigene ID | Description and findings |
|---|---|---|
| 1 | Hs.771 | Phosphorylase, glycogen; liver (Hers disease, |
| | | glycogen storage disease type VI) (PYGL). |
| 2 | Hs.66744 | B-HLH DNA binding protein. H-twist. |
| 3 | Hs.173094 | KIAA1750 |
| 4 | Hs.66052 | CD38 antigen (p45) |
| 5 | Hs.42824 | FLJ10718 hypothetical protein |
| 6 | Hs.139851 | Caveolin 2 (CAV2) |
| 7 | Hs.34045 | FLJ20764 hypothetical protein |
| 8 | Hs.37035 | Homeo box HB9 |

Genes were ranked using the Pearson correlation criterion, see Table 17, with disease progression coded as Normal=1, Dysplasia=2, Grade3=3, Grade4=4. The p values are smaller than in the genes of Table 15, but the AUCs are worse. Three Febbo genes were found, corresponding to genes ranked 6^{th}, 7^{th} and 34^{th}.

TABLE 17

| | Order | | | | Test | Cancer | | |
|---|---|---|---|---|---|---|---|---|
| Rank | num | Unigene ID | Pvalue | FDR | AUC | cor | Febbo | Gene description |
| 1 | 6519 | Hs.243960 | <0.1 | <0.0003 | 0.85 | −1 | 0 | gb: NM_016250.1/DEF = Homo s |
| 2 | 9457 | Hs.128749 | <0.1 | <0.0003 | 0.93 | 1 | 0 | Consensus includes gb: AI796120 |
| 3 | 9976 | Hs.103665 | <0.1 | <0.0003 | 0.89 | −1 | 0 | gb: BC004300.1/DEF = Homo sapiens, |
| 4 | 9459 | Hs.128749 | <0.1 | <0.0003 | 0.87 | 1 | 0 | gb: AF047020.1/DEF = Homo sapiens |
| | | | | | | | | gb: NM_014324.1 |
| 5 | 9458 | Hs.128749 | <0.1 | <0.0003 | 0.89 | 1 | 0 | Consensus includes gb: AA888 |
| 6 | 12337 | Hs.7780 | <0.1 | <0.0003 | 0.96 | 1 | 1 | Consensus includes gb: AV715767 |
| 7 | 893 | Hs.226795 | <0.1 | <0.0003 | 0.97 | −1 | 1 | gb: NM_000852.2/DEF = Homo sapiens |
| 8 | 19589 | Hs.45140 | <0.1 | <0.0003 | 0.98 | −1 | 0 | gb: NM_021637.1/DEF = Homo sapiens |
| 9 | 11911 | Hs.279009 | <0.1 | <0.0003 | 0.98 | −1 | 0 | Consensus includes gb: AI653730 |
| 10 | 17944 | Hs.279905 | <0.1 | <0.0003 | 0.96 | 1 | 0 | gb: NM_016359.1/DEF = Homo sapiens |
| | | | | | | | | gb: AF290612.1 gb: AF090915.1 |
| 11 | 9180 | Hs.239926 | <0.1 | <0.0003 | 0.96 | −1 | 0 | Consensus includes gb: AV704962 |
| 12 | 18122 | Hs.106747 | <0.1 | <0.0003 | 0.96 | −1 | 0 | gb: NM_021626.1/DEF = Homo sapiens |
| | | | | | | | | protein /FL = gb: AF282618.1 gb: NM_{—} |
| 13 | 12023 | Hs.74034 | <0.1 | <0.0003 | 0.96 | −1 | 0 | Consensus includes gb: AU14739 |
| 14 | 374 | Hs.234642 | <0.1 | <0.0003 | 0.96 | −1 | 0 | Cluster Incl. 74607: za55a01.s1 |
| 15 | 12435 | Hs.82432 | <0.1 | <0.0003 | 0.96 | −1 | 0 | Consensus includes b: AA135522 |
| 16 | 18598 | Hs.9728 | <0.1 | <0.0003 | 0.96 | −1 | 0 | gb: NM_016608.1/DEF = Homo sapiens |
| 17 | 3638 | Hs.74120 | <0.1 | <0.0003 | 0.97 | −1 | 0 | gb: NM_006829.1/DEF = Homo sapiens |
| 18 | 5150 | Hs.174151 | <0.1 | <0.0003 | 0.97 | −1 | 0 | gb: NM_001159.2/DEF = Homo sapiens |
| 19 | 1889 | Hs.195850 | <0.1 | <0.0003 | 0.97 | −1 | 0 | gb: NM_000424.1/DEF = Homo |
| | | | | | | | | sapiens/DB_XREF = gi: 4557889/UG = Hs. |
| 20 | 3425 | Hs.77256 | <0.1 | <0.0003 | 0.97 | 1 | 0 | gb: NM_004456.1/DEF = Homo |
| | | | | | | | | sapiens/FL = gb: U61145.1 |
| | | | | | | | | gb: NM_004456.1 |
| 21 | 5149 | Hs.174151 | <0.1 | <0.0003 | 0.96 | −1 | 0 | gb: AB046692.1/DEF = Homo sapiens |
| 22 | 4351 | Hs.303090 | <0.1 | <0.0003 | 0.97 | −1 | 0 | Consensus includes gb: N26005 |
| 23 | 4467 | Hs.24587 | <0.1 | <0.0003 | 0.97 | −1 | 0 | gb: NM_005864.1/DEF = Homo |
| | | | | | | | | sapiens/FL = gb: AB001466.1 |
| | | | | | | | | gb: NM_005864.1 |
| 24 | 12434 | Hs.250723 | <0.1 | <0.0003 | 0.96 | −1 | 0 | Consensus includes gb: BF968134 |
| 25 | 12809 | Hs.169401 | <0.1 | <0.0003 | 0.95 | 1 | 0 | Consensus includes gb: AI358867 |
| 26 | 7082 | Hs.95197 | <0.1 | <0.0003 | 0.95 | −1 | 0 | gb: AB015228.1/DEF = Homo sapiens |
| | | | | | | | | gb: AB015228.1 |
| 27 | 18659 | Hs.73625 | <0.1 | <0.0003 | 0.95 | 1 | 0 | gb: NM_005733.1/DEF = Homo sapiens |
| | | | | | | | | (rabkinesin6)/FL = gb: AF070672.1 |
| 28 | 13862 | Hs.66744 | <0.1 | <0.0003 | 0.98 | 1 | 0 | Consensus includes gb: X99268.1 |
| | | | | | | | | syndrome)/FL = gb: NM_000474 |
| 29 | 3059 | Hs.771 | <0.1 | <0.0003 | 0.98 | −1 | 0 | gb: NM_002863.1/DEF = Homo |
| | | | | | | | | sapiens/DB_XREF = gi: 4506352/UG = Hs. |
| 30 | 15294 | Hs.288649 | <0.1 | <0.0003 | 0.98 | 1 | 0 | Consensus includes gb: AK0 |
| 31 | 9325 | Hs.34853 | <0.1 | <0.0003 | 0.99 | −1 | 0 | Consensus includes gb: AW157094 |
| 32 | 18969 | Hs.20814 | <0.1 | <0.0003 | 0.98 | 1 | 0 | gb: NM_015955.1/DEF = Homo sapiens |
| 33 | 4524 | Hs.65029 | <0.1 | <0.0003 | 0.96 | −1 | 0 | gb: NM_002048.1/DEF = Homo sapiens |
| 34 | 1908 | Hs.692 | <0.1 | <0.0003 | 0.97 | 1 | 1 | gb: NM_002354.1/DEF = Homo sapiens |
| | | | | | | | | signal transducer 1/FL = gb: M32306.1 |
| 35 | 11407 | Hs.326776 | <0.1 | <0.0003 | 0.96 | −1 | 0 | gb: AF180519.1/DEF = Homo sapiens |
| | | | | | | | | cds/FL = gb: AF180519.1 |
| 36 | 19501 | Hs.272813 | <0.1 | <0.0003 | 0.96 | −1 | 0 | gb: NM_017434.1/DEF = Homo sapiens |
| 37 | 11248 | Hs.17481 | <0.1 | <0.0003 | 0.96 | −1 | 0 | gb: AF063606.1/DEF = Homo sapiens |
| 38 | 5894 | Hs.80247 | <0.1 | <0.0003 | 0.95 | −1 | 0 | gb: NM_000729.2/DEF = Homo sapiens |
| 39 | 19455 | Hs.26892 | <0.1 | <0.0003 | 0.96 | −1 | 0 | gb: NM_018456.1/DEF = Homo sapie |
| | | | | | | | | BM040/FL = gb: AF217516.1 gb: |
| 40 | 3448 | Hs.169401 | <0.1 | <0.0003 | 0.96 | 1 | 0 | Consensus includes gb: N33009 |
| 41 | 6666 | Hs.90911 | <0.1 | <0.0003 | 0.96 | −1 | 0 | gb: NM_004695.1/DEF = Homo |
| | | | | | | | | sapiens/UG = Hs.90911 solute carrier |
| | | | | | | | | family |
| 42 | 6924 | Hs.820 | <0.1 | <0.0003 | 0.98 | 1 | 0 | gb: NM_004503.1/DEF = Homo sapiens |
| 43 | 2169 | Hs.250811 | <0.1 | <0.0003 | 0.98 | −1 | 0 | Consensus includes gb: BG169673 |
| 44 | 12168 | Hs.75318 | <0.1 | <0.0003 | 0.98 | −1 | 0 | Consensus includes gb: AL565074 |
| 45 | 18237 | Hs.283719 | <0.1 | <0.0003 | 0.98 | −1 | 0 | gb: NM_018476.1/DEF = Homo sapiens |
| | | | | | | | | HBEX2/FL = gb: AF220189.1 gb: |
| 46 | 5383 | Hs.182575 | <0.1 | <0.0003 | 0.98 | −1 | 0 | Consensus includes gb: BF223679 |
| 47 | 19449 | Hs.17296 | <0.1 | <0.0003 | 0.99 | −1 | 0 | gb: NM_023930.1/DEF = Homo sapiens |
| | | | | | | | | gb: BC001929.1 gb: NM_023930.1 |
| 48 | 4860 | Hs.113082 | <0.1 | <0.0003 | 0.99 | −1 | 0 | gb: NM_014710.1/DEF = Homo sapiens |
| 49 | 17714 | Hs.5216 | <0.1 | <0.0003 | 0.99 | 1 | 0 | gb: NM_014038.1/DEF = Homo sapiens |
| 50 | 12020 | Hs.137476 | <0.1 | <0.0003 | 0.97 | −1 | 0 | Consensus includes gb: AL582836 |

The data is rich in potential biomarkers. To find the most promising markers, criteria were designed to implement prior knowledge of disease severity and zonal information. This allowed better separation of relevant genes from genes that coincidentally well separate the data, thus alleviating the problem of overfitting. To further reduce the risk of overfitting, genes were selected that were also found in an independent study Table 15. Those genes include well-known proteins involved in prostate cancer and some potentially interesting targets.

### Example 5

### Prostate Cancer Gene Expression Microarray Data (11-2004)

Separations of class pairs were performed for “tumor (G3+4) vs. all other tissues”. These separations are relatively easy and can be performed with fewer than 10 genes, however, hundreds of significant genes were identified.

Separations of “G4 vs. all others”, “Dysplasia vs. all others”, and “Normal vs. all others” are less easy (best AUCs between 0.75 and 0.85) and separation of “G3 vs. all others” is almost impossible in this data (AUC around 0.5). With over 100 genes, G4 can be separated from all other tissues with about 10% BER. Hundreds of genes separate G4 from all other tissues significantly, yet one cannot find a good separation with just a few genes.

Separations of “TZG4 vs. PZG4”, “Normal vs. Dysplasia” and “G3 vs. G4” are also hard. 10×10-fold CV yielded very poor results. Using leave-one out CV and under 20 genes, we separated some pairs of classes: ERR_{TZG4/PZG4}≈6%, ERR_{NL/Dys} and ERR_{G3/G4}≈9%. However, due to the small sample sizes, the significance of the genes found for those separations is not good, shedding doubt on the results.

Pre-operative PSA was found to correlate poorly with clinical variables (R²=0.316 with cancer volume, 0.025 with prostate weight, and 0.323 with CAvol/Weight). Genes were found with activity that correlated with pre-operative PSA either in BPH samples or G34 samples or both. Possible connections of those genes were found to cancer and/or prostate in the literature, but their relationship to PSA is not documented. Genes associated to PSA by their description do not have expression values correlated with pre-operative PSA. This illustrates that gene expression coefficients do not necessarily reflect the corresponding protein abundance.

Genes were identified that correlate with cancer volume in G3+4 tissues and with cure/fail prognosis. Neither are statistically significant, however, the gene most correlated with cancer volume has been reported in the literature as connected to prostate cancer. Prognosis information can be used in conjunction with grade levels to determine the significance of genes. Several genes were identified for separating G4 from non-G4 and G3 from G3 in the group the samples of patients with the poor prognosis in regions of lowest expression values.

The following experiments were performed using data consisting of a matrix of 87 lines (samples) and 22283 columns (genes) obtained from an Affymetrix U133A GeneChip®. The distributions of the samples of the microarray prostate cancer study are the same as those listed in Table 12.

Genes were selected on the basis of their individual separating power, as measured by the AUC (area under the ROC curve that plots sensitivity vs. specificity).

Similarly “random genes” that are genes obtained by permuting randomly the values of columns of the matrix are ranked. Where N is the total number of genes (here, N=22283, 40 times more random genes than real genes are used to estimate p values accurately (Nᵣ=40*22283). For a given AUC value A, nᵣ(A) is the number of random genes that have an AUC larger than A. The p value is estimated by the fraction of random genes that have an AUC larger than A, i.e.,:

Pvalue=(1+nᵣ(A))/Nᵣ

Adding 1 to the numerator avoids having zero p values for the best ranking genes and accounts for the limited precision due to the limited number of random genes. Because the pvalues of a large number of genes are measured simultaneously, correction must be applied to account for this multiple testing. As in the previous example, the simple Bonferroni correction is used:

Bonferroni_{—}pvalue=N*(1+nᵣ(A))/Nᵣ

Hence, with a number of probes that is 40 times the number of genes, the p values are estimated with an accuracy of 0.025.

For a given gene of AUC value A, one can also compute the false discovery rate (FDR), which is an estimate of the ratio of the number of falsely significant genes over the number of genes called significant. Where n(A) is the number of genes found above A, the FDR is computed as the ratio of the p value (before Bonferroni correction) and the fraction of real genes found above A:

FDR=pvalue*N/n(A)=((1+nᵣ(A))*N)/(n(A)*Nᵣ).

Linear ridge regression classifiers (similar to SVMs) were trained with 10×10-fold cross validation, i.e., the data were split 100 times into a training set and a test set and the average performance and standard deviation were computed. In these experiments, the feature selection is performed within the cross-validation loop. That is, a separate featuring ranking is performed for each data split. The number of features are varied and a separate training/testing is performed for each number of features. Performances for each number of features are averaged to plot performance vs. number of features. The ridge value is optimized separately for each training subset and number of features, using the leave-one-out error, which can be computed analytically from the training error. In some experiments, the 10×10-fold cross-validation was done by leave-one-out cross-validation. Everything else remains the same.

Using the rankings obtained for the 100 data splits of the machine learning experiments (also called “bootstraps”), average gene ranks are computed. Average gene rank carries more information in proportion to the fraction of time a gene was always found in the top N ranking genes. This last criterion is sometimes used in the literature, but the number of genes always found in the top N ranking genes appears to grows linearly with N.

The following statistics were computed for cross-validation (10 times 10-fold or leave-one-out) of the machine learning experiments:

AUC mean: The average area under the ROC curve over all data splits.

AUC stdev: The corresponding standard deviation. Note that the standard error obtained by dividing stdev by the square root of the number of data splits is inaccurate because sampling is done with replacements and the experiments are not independent of one another.

BER mean: The average BER over all data splits. The BER is the balanced error rate, which is the average of the error rate of examples of the first class and examples of the second class. This provides a measure that is not biased toward the most abundant class.

BER stdev: The corresponding standard deviation.

Pooled AUC: The AUC obtained using the predicted classification values of all the test examples in all data splits altogether.

Pooled BER: The BER obtained using the predicted classification values of all the test examples in all data splits altogether.

Note that for leave-one-out CV, it does not make sense to compute BER-mean because there is only one example in each test set. Instead, the leave-one-out error rate or the pooled BER is computed.

High classification accuracy (as measured by the AUC) can be achieved a small number of genes (3 or more) to provide an AUC above 0.90. If the experimental repeats were independent, the standard error of the mean obtained by dividing the standard deviation by 10 could be used as an error bar. A more reasonable estimate of the error bar may be obtained by dividing it by three to account for the dependencies between repeats.

The genes listed in the following tables are ranked according to their individual AUC computed with all the data. The first column is the rank, followed by the Gene ID (order number in the data matrix), and the Unigene ID. The column “Under Expr” is +1 if the gene is underexpressed and −1 otherwise. AUC is the ranking criterion. Pval is the pvalue computed with random genes as explained above. FDR is the false discovery rate. “Ave. rank” is the average rank of the feature when subsamples of the data are taken in a 10×10-fold cross-validation experiment in Tables 18, 21, 23, 25 & 27 and with leave-one-out in Tables 29, 31 & 33.

In the test to separate tumors (cancer G3 and G4) from other tissues, the results show that it is relatively easy to separate tumor from other tissues. The list of the top 50 tumor genes, both overexpressed and underexpressed in cancer, is shown in Table 18. A complete listing of the top 200 tumor genes is provided in FIG. The three best genes, Gene IDs no. 9457, 9458 and 9459 all have same Unigene ID. Additional description about the top three genes is provided in Table 19 below.

TABLE 18

| | | | Under | | | | |
|---|---|---|---|---|---|---|---|
| | Gene | | Expr. In | | | | Ave. |
| Rank | ID | Unigene ID | tumor | AUC | Pval | FDR | rank |
| 1 | 9459 | Hs.128749 | −1 | 0.9458 | 0.02 | 0.025 | 1.16 |
| 2 | 9458 | Hs.128749 | −1 | 0.9425 | 0.02 | 0.012 | 2.48 |
| 3 | 9457 | Hs.128749 | −1 | 0.9423 | 0.02 | 0.0083 | 2.51 |
| 4 | 11911 | Hs.279009 | 1 | 0.9253 | 0.02 | 0.0062 | 4.31 |
| 5 | 12337 | Hs.7780 | −1 | 0.9125 | 0.02 | 0.005 | 7.23 |
| 6 | 983 | Hs.226795 | 1 | 0.9076 | 0.02 | 0.0042 | 8.42 |
| 7 | 18792 | Hs.6823 | −1 | 0.9047 | 0.02 | 0.0036 | 10.04 |
| 8 | 1908 | Hs.692 | −1 | 0.9044 | 0.02 | 0.0031 | 10.03 |
| 9 | 19589 | Hs.45140 | 1 | 0.9033 | 0.02 | 0.0028 | 10.47 |
| 10 | 6519 | Hs.243960 | 1 | 0.8996 | 0.02 | 0.0025 | 12.67 |
| 11 | 17714 | Hs.5216 | −1 | 0.8985 | 0.02 | 0.0023 | 13.93 |
| 12 | 18122 | Hs.106747 | 1 | 0.8985 | 0.02 | 0.0021 | 13.86 |
| 13 | 18237 | Hs.283719 | 1 | 0.8961 | 0.02 | 0.0019 | 16.61 |
| 14 | 3059 | Hs.771 | 1 | 0.8942 | 0.02 | 0.0018 | 17.86 |
| 15 | 16533 | Hs.110826 | −1 | 0.8921 | 0.02 | 0.0017 | 19.44 |
| 16 | 18598 | Hs.9728 | 1 | 0.8904 | 0.02 | 0.0016 | 19.43 |
| 17 | 12434 | Hs.250723 | 1 | 0.8899 | 0.02 | 0.0015 | 20.19 |
| 18 | 4922 | Hs.55279 | 1 | 0.884 | 0.02 | 0.0014 | 27.23 |
| 19 | 13862 | Hs.66744 | −1 | 0.8832 | 0.02 | 0.0013 | 30.59 |
| 20 | 9976 | Hs.103665 | 1 | 0.8824 | 0.02 | 0.0012 | 30.49 |
| 21 | 18835 | Hs.44278 | −1 | 0.8824 | 0.02 | 0.0012 | 30.94 |
| 22 | 3331 | Hs.54697 | 1 | 0.8802 | 0.02 | 0.0011 | 32.35 |
| 23 | 18969 | Hs.20814 | −1 | 0.8797 | 0.02 | 0.0011 | 35.89 |
| 24 | 9373 | Hs.21293 | −1 | 0.8786 | 0.02 | 0.001 | 35.52 |
| 25 | 15294 | Hs.288649 | −1 | 0.8786 | 0.02 | 0.001 | 35.69 |
| 26 | 4497 | Hs.33084 | 1 | 0.8776 | 0.02 | 0.00096 | 37.77 |
| 27 | 5001 | Hs.823 | −1 | 0.8765 | 0.02 | 0.00093 | 40.25 |
| 28 | 9765 | Hs.22599 | 1 | 0.8765 | 0.02 | 0.00089 | 39.32 |
| 29 | 4479 | Hs.198760 | 1 | 0.8759 | 0.02 | 0.00086 | 40.82 |
| 30 | 239 | Hs.198760 | 1 | 0.8749 | 0.02 | 0.00083 | 43.04 |
| 31 | 6666 | Hs.90911 | 1 | 0.8749 | 0.02 | 0.00081 | 42.53 |
| 32 | 12655 | Hs.10587 | 1 | 0.8749 | 0.02 | 0.00078 | 41.56 |
| 33 | 19264 | Hs.31608 | −1 | 0.8743 | 0.02 | 0.00076 | 44.66 |
| 34 | 5923 | Hs.171731 | 1 | 0.8738 | 0.02 | 0.00074 | 44.3 |
| 35 | 1889 | Hs.195850 | 1 | 0.8727 | 0.02 | 0.00071 | 46.1 |
| 36 | 21568 | Hs.111676 | 1 | 0.8716 | 0.02 | 0.00069 | 48.3 |
| 37 | 3264 | Hs.139336 | −1 | 0.8714 | 0.02 | 0.00068 | 51.17 |
| 38 | 14738 | Hs.8198 | 1 | 0.8706 | 0.02 | 0.00066 | 52.7 |
| 39 | 1867 | Hs.234680 | 1 | 0.8695 | 0.02 | 0.00064 | 52.99 |
| 40 | 4467 | Hs.24587 | 1 | 0.8695 | 0.02 | 0.00062 | 52.25 |
| 41 | 9614 | Hs.8583 | 1 | 0.8695 | 0.02 | 0.00061 | 53.62 |
| 42 | 18659 | Hs.73625 | −1 | 0.8692 | 0.02 | 0.0006 | 56.86 |
| 43 | 20137 | Hs.249727 | 1 | 0.8692 | 0.02 | 0.00058 | 55.2 |
| 44 | 12023 | Hs.74034 | 1 | 0.869 | 0.02 | 0.00057 | 55.69 |
| 45 | 12435 | Hs.82432 | 1 | 0.869 | 0.02 | 0.00056 | 56.63 |
| 46 | 14626 | Hs.23960 | −1 | 0.8687 | 0.02 | 0.00054 | 58.95 |
| 47 | 7082 | Hs.95197 | 1 | 0.8684 | 0.02 | 0.00053 | 56.27 |
| 48 | 15022 | Hs.110826 | −1 | 0.8679 | 0.02 | 0.00052 | 59.51 |
| 49 | 20922 | Hs.0 | −1 | 0.8679 | 0.02 | 0.00051 | 59.93 |
| 50 | 4361 | Hs.102 | 1 | 0.8673 | 0.02 | 0.0005 | 60.94 |

TABLE 19

| Gene ID | Description |
|---|---|
| 9457 | gb: AI796120 /FEA = EST /DB_XREF = |
| | gi: 5361583 /DB_XREF = est: wh42f03.x1 |
| | /CLONE = IMAGE: 2383421 /UG = |
| | Hs.128749 alphamethylacyl-CoA racemase |
| | /FL = gb: AF047020.1 gb: AF158378.1 gb: NM_014324.1 |
| 9458 | gb: AA888589 /FEA = EST /DB_XREF = |
| | gi: 3004264 /DB_XREF = est: oe68e10.s1 |
| | /CLONE = IMAGE: 1416810 /UG = |
| | Hs.128749 alphamethylacyl-CoA racemase |
| | /FL = gb: AF047020.1 gb: AF158378.1 gb: NM_014324.1 |
| 9459 | gb: AF047020.1 /DEF = |
| | Homo sapiens alpha-methylacyl-CoA racemase mRNA, |
| | complete cds. /FEA = mRNA /PROD = |
| | alpha-methylacyl-CoA racemase |
| | /DB_XREF = gi: 4204096 /UG = |
| | Hs.128749 alpha-methylacyl-CoA racemase |
| | /FL = gb: AF047020.1 gb: AF158378.1 gb: NM_014324.1 |

This gene has been reported in numerous papers including Luo, et al., Molecular Carcinogenesis, 33(1): 25-35 (January 2002); Luo J, et al., Abstract Cancer Res., 62(8): 2220-6 (2002 Apr. 15).

Table 20 shows the separation with varying number of features for tumor (G3+4) vs. all other tissues.

TABLE 20

| | feat. num. | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 16 | 32 | 64 | 128 |
| 100 * | 92.28 | 93.33 | 93.83 | 94 | 94.33 | 94.43 | 94.1 | 93.8 | 93.43 | 93.53 | 93.45 | 93.37 | 93.18 | 93.03 |
| AUC | | | | | | | | | | | | | | |
| 100 * | 11.73 | 10.45 | 10 | 9.65 | 9.63 | 9.61 | 10.3 | 10.54 | 10.71 | 10.61 | 10.75 | 10.44 | 11.49 | 11.93 |
| AUCstd | | | | | | | | | | | | | | |
| BER | 14.05 | 13.1 | 12.6 | 10.25 | 9.62 | 9.72 | 9.75 | 9.5 | 9.05 | 9.05 | 9.7 | 9.6 | 10.12 | 9.65 |
| (%) | | | | | | | | | | | | | | |
| BERstd | 13.51 | 12.39 | 12.17 | 11.77 | 9.95 | 10.06 | 10.15 | 10.04 | 9.85 | 10.01 | 10.2 | 10.3 | 10.59 | 10.26 |
| (%) | | | | | | | | | | | | | | |

Using the same experimental setup, separations were attempted for G4 from non G4, G3 from non G3, Dysplasia from non-dys and Normal from non-Normal. These separations were less successful than the above-described tests, indicating that G3, dysplasia and normal do not have molecular characteristics that distinguish them easily from all other samples. Lists of genes are provided in Tables 21-37.

Table 21 lists the top 10 genes separating Grade 4 prostate cancer (G4) from all others.

TABLE 21

| | | | Under | | | | |
|---|---|---|---|---|---|---|---|
| | Gene | | Expr. In | | | | Ave. |
| Rank | ID | Unigene ID | G4 | AUC | Pval | FDR | rank |
| 1 | 5923 | Hs.171731 | 1 | 0.9204 | 0.02 | 0.025 | 3.25 |
| 2 | 18122 | Hs.106747 | 1 | 0.9136 | 0.02 | 0.012 | 6.17 |
| 3 | 19573 | Hs.232165 | 1 | 0.9117 | 0.02 | 0.0083 | 7.92 |
| 4 | 893 | Hs.226795 | 1 | 0.9099 | 0.02 | 0.0062 | 7.22 |
| 5 | 9889 | Hs.137569 | 1 | 0.9093 | 0.02 | 0.005 | 8.8 |
| 6 | 19455 | Hs.26892 | 1 | 0.908 | 0.02 | 0.0042 | 10.54 |
| 7 | 19589 | Hs.45140 | 1 | 0.9074 | 0.02 | 0.0036 | 10.54 |
| 8 | 18598 | Hs.9728 | 1 | 0.9062 | 0.02 | 0.0031 | 10.83 |
| 9 | 6519 | Hs.243960 | 1 | 0.9037 | 0.02 | 0.0028 | 12.79 |
| 10 | 11175 | Hs.137569 | 1 | 0.9031 | 0.02 | 0.0025 | 13.46 |

Table 22 below provides the details for the top two genes of this group.

TABLE 22

| Gene ID | Description |
|---|---|
| 5923 | gb: NM_015865.1 /DEF = |
| | Homo sapiens solute carrier family 14 (urea transporter), |
| | member 1 (Kidd blood group) (SLC14A1), |
| | mRNA. /FEA = mRNA |
| | /GEN = SLC14A1 /PROD = RACH1 /DB_XREF = |
| | gi: 7706676 /UG = Hs.171731 |
| | solute carrier family 14 (urea transporter), member 1 |
| | (Kidd blood group) |
| | /FL = gb: U35735.1 gb: NM_015865.1 |
| 18122 | gb: NM_021626.1 /DEF = Homo sapiens |
| | serine carboxypeptidase 1 precursor |
| | protein (HSCP1), mRNA. /FEA = mRNA /GEN = |
| | HSCP1 /PROD = serine |
| | carboxypeptidase 1 precursor protein /DB_XREF = |
| | gi: 11055991 /UG = Hs.106747 |
| | serine carboxypeptidase 1 precursor protein /FL = gb: |
| | AF282618.1 |
| | gb: NM_021626.1 gb: AF113214.1 gb: AF265441.1 |

The following provide the gene descriptions for the top two genes identified in each separation:

Table 23 lists the top 10 genes separating Normal prostate versus all others.

TABLE 23

| | | | Under | | | | |
|---|---|---|---|---|---|---|---|
| | | | Expr. | | | | |
| | Gene | | in | | | | Ave. |
| Rank | ID | Unigene ID | Normal | AUC | Pval | FDR | Rank |
| 1 | 6519 | Hs.243960 | −1 | 0.886 | 0.02 | 0.025 | 1.3 |
| 2 | 3448 | Hs.169401 | 1 | 0.8629 | 0.02 | 0.012 | 4.93 |
| 3 | 17900 | Hs.8185 | −1 | 0.8601 | 0.02 | 0.0083 | 6.17 |
| 4 | 6666 | Hs.90911 | −1 | 0.8552 | 0.02 | 0.0062 | 6.59 |
| 5 | 893 | Hs.226795 | −1 | 0.8545 | 0.02 | 0.005 | 7.22 |
| 6 | 6837 | Hs.159330 | −1 | 0.8545 | 0.02 | 0.0042 | 8.05 |
| 7 | 374 | Hs.234642 | −1 | 0.8483 | 0.02 | 0.0036 | 9.69 |
| 8 | 9976 | Hs.103665 | −1 | 0.8458 | 0.02 | 0.0031 | 11.62 |
| 9 | 3520 | Hs.2794 | −1 | 0.8399 | 0.02 | 0.0028 | 15.29 |
| 10 | 3638 | Hs.74120 | −1 | 0.8357 | 0.02 | 0.0025 | 18.17 |

The top two genes from Table 23 are described in detail in Table 24.

TABLE 24

| Gene ID | Description |
|---|---|
| 6519 | gb: NM_016250.1 /DEF = Homo sapiens |
| | N-myc downstream-regulated gene 2 |
| | (NDRG2), mRNA. /FEA = mRNA /GEN = |
| | NDRG2 /PROD = KIAA1248 protein |
| | /DB_XREF = gi: 10280619 /UG = Hs.243960 |
| | N-myc downstream-regulated gene 2 |
| | /FL = gb: NM_016250.1 gb: AF159092. |
| 3448 | gb: N33009 /FEA = EST /DB_XREF = |
| | gi: 1153408 /DB_XREF = est: yy31f09.s1 |
| | /CLONE = IMAGE: 272873 /UG = Hs.169401 |
| | apolipoprotein E |
| | /FL = gb: BC003557.1 gb: M12529.1 gb: K00396.1 |
| | gb: NM_000041.1 |

Table 25 lists the top 10 genes separating G3 prostate cancer from all others.

TABLE 25

| | | | Under | | | | |
|---|---|---|---|---|---|---|---|
| | Gene | | Expr. in | | | | Ave. |
| Rank | ID | Unigene ID | G3 | AUC | Pval | FDR | rank |
| 1 | 18446 | Hs.283683 | −1 | 0.8481 | 1 | 1.5 | 2.14 |
| 2 | 2778 | Hs.230 | −1 | 0.8313 | 1 | 1.8 | 8.14 |
| 3 | 16102 | Hs.326526 | 1 | 0.8212 | 1 | 2.2 | 10.71 |
| 4 | 12046 | Hs.166982 | 1 | 0.817 | 1 | 2.1 | 15.14 |
| 5 | 9156 | Hs.3416 | −1 | 0.8158 | 1 | 1.8 | 14.71 |
| 6 | 9459 | Hs.128749 | −1 | 0.8158 | 1 | 1.5 | 20.43 |
| 7 | 21442 | Hs.71819 | −1 | 0.8158 | 1 | 1.3 | 13.86 |
| 8 | 6994 | Hs.180248 | −1 | 0.814 | 1 | 1.3 | 11.71 |
| 9 | 17019 | Hs.128749 | −1 | 0.8116 | 1 | 1.3 | 23.14 |
| 10 | 9457 | Hs.128749 | −1 | 0.8074 | 1 | 1.3 | 34.71 |

The top two genes listed in Table 25 are described in detail in Table 26.

TABLE 26

| Gene ID | Description |
|---|---|
| 18446 | gb: NM_020130.1 /DEF = |
| | Homo sapiens chromosome 8 open reading frame 4 |
| | (C8ORF4), mRNA. /FEA = mRNA /GEN = |
| | C8ORF4 /PROD = chromosome 8 open |
| | reading frame 4 /DB_XREF = |
| | gi: 9910147 /UG = Hs.283683 chromosome 8 open |
| | reading frame 4 /FL = gb: AF268037.1 gb: NM_020130.1 |
| 2778 | gb: NM_002023.2 /DEF = |
| | Homo sapiens fibromodulin (FMOD), mRNA. |
| | /FEA = mRNA /GEN = FMOD /PROD = fibromodulin |
| | precursor |
| | /DB_XREF = gi: 5016093 /UG = |
| | Hs.230 fibromodulin /FL = gb: NM_002023.2 |

Table 27 shows the top 10 genes separating Dysplasia from everything else.

TABLE 27

| | | | Under | | | | |
|---|---|---|---|---|---|---|---|
| | Gene | | Expr. in | | | | Ave. |
| Rank | ID | Unigene ID | dysplasia | AUC | Pval | FDR | rank |
| 1 | 5509 | Hs.178121 | −1 | 0.8336 | 0.15 | 0.15 | 4.53 |
| 2 | 4102 | Hs.75426 | −1 | 0.8328 | 0.15 | 0.075 | 4.31 |
| 3 | 10777 | Hs.101047 | 1 | 0.8319 | 0.17 | 0.058 | 5.6 |
| 4 | 18814 | Hs.319088 | 1 | 0.8189 | 0.45 | 0.11 | 10.95 |
| 5 | 4450 | Hs.154879 | 1 | 0.8168 | 0.5 | 0.1 | 11.57 |
| 6 | 14885 | Hs.2554 | 1 | 0.8164 | 0.53 | 0.088 | 18.04 |
| 7 | 10355 | Hs.169832 | 1 | 0.8126 | 0.63 | 0.089 | 14.3 |
| 8 | 5072 | Hs.122647 | −1 | 0.8063 | 0.72 | 0.091 | 26.77 |
| 9 | 3134 | Hs.323469 | −1 | 0.805 | 0.8 | 0.089 | 22.76 |
| 10 | 15345 | Hs.95011 | 1 | 0.8017 | 1 | 0.11 | 29.3 |

Table 28 provides the details for the top two genes listed in Table 27.

TABLE 28

| Gene ID | Description |
|---|---|
| 5509 | gb: NM_021647.1 /DEF = |
| | Homo sapiens KIAA0626 gene product (KIAA0626), |
| | mRNA. /FEA = mRNA /GEN = |
| | KIAA0626 /PROD = KIAA0626 gene product |
| | /DB_XREF = gi: 11067364 /UG = |
| | Hs.178121 KIAA0626 gene product |
| | /FL = gb: NM_021647.1 gb: AB014526.1 |
| 4102 | gb: NM_003469.2 /DEF = |
| | Homo sapiens secretogranin II (chromogranin C) |
| | (SCG2), mRNA. /FEA = mRNA /GEN = SCG2 /PROD = |
| | secretogranin II precursor |
| | /DB_XREF = gi: 10800415 /UG = Hs.75426 |
| | secretogranin II (chromogranin C) |
| | /FL = gb: NM_003469.2 gb: M25756.1 |

Due to the small sample sizes, poor performance was obtained with 10×10-fold cross-validation. To avoid this problem, leave-one-out cross-validation was used instead. In doing so, the average AUC for all repeats cannot be reported because there is only one test example in each repeat. Instead, the leave-one-out error rate and the pooled AUC are evaluated. However, all such pairwise separations are difficult to achieve with high accuracy and a few features.

Table 29 lists the top 10 genes separating G3 from G4. Table 30 provides the details for the top two genes listed.

TABLE 29

| | | | (+) Expr. | | | | |
|---|---|---|---|---|---|---|---|
| | | | in G4; | | | | |
| | Gene | | (−) Expr. | | | | Ave. |
| Rank | ID | Unigene ID | in G3 | AUC | Pval | FDR | rank |
| 1 | 19455 | Hs.26892 | −1 | 0.9057 | 0.45 | 0.45 | 1.09 |
| 2 | 11175 | Hs.137569 | −1 | 0.8687 | 1 | 1.8 | 2.95 |
| 3 | 9156 | Hs.3416 | −1 | 0.8653 | 1 | 1.4 | 4 |
| 4 | 18904 | Hs.315167 | 1 | 0.8653 | 1 | 1.1 | 4.71 |
| 5 | 9671 | Hs.98658 | 1 | 0.8636 | 1 | 0.99 | 5.45 |
| 6 | 2338 | Hs.62661 | −1 | 0.8586 | 1 | 0.96 | 6.64 |
| 7 | 2939 | Hs.82906 | 1 | 0.8586 | 1 | 0.82 | 7.46 |
| 8 | 450 | Hs.27262 | 1 | 0.8552 | 1 | 0.8 | 8.44 |
| 9 | 18567 | Hs.193602 | 1 | 0.8535 | 1 | 0.85 | 9.49 |
| 10 | 5304 | Hs.252136 | −1 | 0.8519 | 1 | 0.77 | 10.67 |

TABLE 30

| Gene | |
|---|---|
| ID | Description |
| 19455 | gb: NM_018456.1 /DEF = |
| | Homo sapiens uncharacterized bone marrow protein |
| | BM040 (BM040), mRNA. /FEA = mRNA /GEN = BM040 |
| | /PROD = uncharacterized bone marrow protein |
| | BM040 /DB_XREF = gi: 8922098 |
| | /UG = Hs.26892 uncharacterized bone marrow protein BM040 |
| | /FL = gb: AF217516.1 gb: NM_018456.1 |
| 11175 | gb: AB010153.1 /DEF = |
| | Homo sapiens mRNA for p73H, complete cds. |
| | /FEA = mRNA /GEN = p73H /PROD = |
| | p73H /DB_XREF = gi: 3445483 |
| | /UG = Hs.137569 tumor protein 63 kDa with strong |
| | homology to p53 /FL = gb: AB010153.1 |

Table 31 lists the top 10 genes for separating Normal prostate from Dysplasia. Details of the top two genes for performing this separation are provided in Table 32.

TABLE 31

| | | | (−) Expr. | | | | |
|---|---|---|---|---|---|---|---|
| | | | in NL; | | | | |
| | Gene | | (+) Expr. | | | | Ave. |
| Rank | ID | Unigene ID | in Dys | AUC | Pval | FDR | rank |
| 1 | 4450 | Hs.154879 | −1 | 0.9037 | 0.05 | 0.05 | 1.09 |
| 2 | 10611 | Hs.41682 | 1 | 0.8957 | 0.075 | 0.037 | 2.02 |
| 3 | 9048 | Hs.177556 | −1 | 0.8743 | 0.45 | 0.15 | 3.17 |
| 4 | 18069 | Hs.103147 | −1 | 0.8717 | 0.57 | 0.14 | 4.06 |
| 5 | 7978 | Hs.20815 | −1 | 0.8583 | 1 | 0.23 | 5.56 |
| 6 | 6837 | Hs.159330 | −1 | 0.8556 | 1 | 0.21 | 6.37 |
| 7 | 7229 | Hs.71816 | −1 | 0.8463 | 1 | 0.34 | 8.03 |
| 8 | 21059 | Hs.283753 | 1 | 0.8449 | 1 | 0.3 | 9.51 |
| 9 | 15345 | Hs.95011 | −1 | 0.8436 | 1 | 0.29 | 9.94 |
| 10 | 2463 | Hs.91251 | −1 | 0.8369 | 1 | 0.38 | 11.78 |

TABLE 32

| Gene ID | Description |
|---|---|
| 4450 | gb: NM_022719.1 /DEF = |
| | Homo sapiens DiGeorge syndrome critical region gene |
| | DGSI (DGSI), mRNA. /FEA = mRNA /GEN = |
| | DGSI /PROD = DiGeorge syndrome |
| | critical region gene DGSIprotein /DB_XREF = |
| | gi: 13027629 /UG = Hs.154879 |
| | DiGeorge syndrome critical region gene DGSI /FL = |
| | gb: NM_022719.1 |
| 10611 | gb: U30610.1 /DEF = Human CD94 protein mRNA, |
| | complete cds. /FEA = mRNA |
| | /PROD = CD94 protein /DB_XREF = |
| | gi: 1098616 /UG = Hs.41682 killer cell lectin- |
| | like receptor subfamily D, member 1 /FL = |
| | gb: U30610.1 gb: NM_002262.2 |

Table 33 lists the top 10 genes for separating peripheral zone G4 prostate cancer from transition zone G4 cancer. Table 34 provides the details for the top two genes in this separation.

TABLE 33

| | | | (−) Expr. | | | | |
|---|---|---|---|---|---|---|---|
| | | | in TZ; | | | | |
| | Gene | | (+) Expr. | | | | Ave. |
| Rank | ID | Unigene ID | In PZ | AUC | Pval | FDR | rank |
| 1 | 4654 | Hs.194686 | 1 | 0.9444 | 1 | 1.2 | 1.1 |
| 2 | 14953 | Hs.306423 | 1 | 0.9306 | 1 | 1.1 | 2.45 |
| 3 | 929 | Hs.279949 | −1 | 0.9167 | 1 | 1.7 | 4 |
| 4 | 6420 | Hs.274981 | 1 | 0.9167 | 1 | 1.3 | 4.84 |
| 5 | 7226 | Hs.673 | 1 | 0.9167 | 1 | 1 | 5.69 |
| 6 | 18530 | Hs.103291 | 1 | 0.9167 | 1 | 0.86 | 6.68 |
| 7 | 6618 | Hs.2563 | 1 | 0.9097 | 1 | 1.1 | 7.82 |
| 8 | 16852 | Hs.75626 | 1 | 0.9097 | 1 | 0.93 | 8.91 |
| 9 | 19242 | Hs.12692 | 1 | 0.9097 | 1 | 0.82 | 9.78 |
| 10 | 6106 | Hs.56294 | 1 | 0.9063 | 1 | 1 | 10.75 |

TABLE 34

| Gene ID | Description |
|---|---|
| 4654 | gb: NM_003951.2 /DEF = |
| | Homo sapiens solute carrier family 25 (mitochondrial |
| | carrier, brain), member 14 (SLC25A14), transcript variant |
| | long, nuclear gene encoding mitochondrial protein, mRNA. |
| | /FEA = mRNA /GEN = SLC25A14 |
| | /PROD = solute carrier family 25, member 14, isoformUCP5L |
| | /DB_XREF = gi: 6006039 /UG = |
| | Hs.194686 solute carrier family 25 (mitochondrial |
| | carrier, brain), member 14 /FL = gb: AF155809.1 |
| | gb: AF155811.1 gb: NM_022810.1 gb: |
| | AF078544.1 gb: NM_003951.2 |
| 14953 | gb: AK002179.1 /DEF = |
| | Homo sapiens cDNA FLJ11317 fis, clone |
| | PLACE1010261, moderately similar to SEGREGATION |
| | DISTORTER PROTEIN. /FEA = mRNA /DB_XREF = |
| | gi: 7023899 /UG = Hs.306423 Homo |
| | sapiens cDNA FLJ11317 fis, clone PLACE1010261, |
| | moderately similar to SEGREGATION DISTORTER |
| | PROTEIN |

As stated in an earlier discussion, PSA is not predictive of tissue malignancy.

There is very little correlation of PSA and cancer volume (R2=0.316). The R2 was also computed for PSA vs. prostate weight (0.025) and PSA vs. CA/Weight (0.323). PSA does not separate well the samples in malignancy categories. In this data, there did not appear to be any correlation between PSA and prostate weight.

A test was conducted to identify the genes most correlated with PSA, in BPH samples or in G3/4 samples, which were found to be genes 11541 for BPH and 14523 for G3/4. The details for these genes are listed below in Table 35.

TABLE 35

| Gene ID | Description |
|---|---|
| 11541 | gb: AB050468.1 /DEF = |
| | Homo sapiens mRNA for membrane glycoprotein LIG-1, |
| | complete cds. /FEA = mRNA /GEN = |
| | lig-1 /PROD = membrane glycoprotein LIG-1 |
| | /DB_XREF = gi: 13537354 /FL = |
| | gb: AB050468.1 |
| 14523 | gb: AL046992 /FEA = EST /DB_XREF = gi: 5435048 |
| | /DB_XREF = est: DKFZp586L0417_r1 /CLONE = |
| | DKFZp586L0417 /UG = Hs.184907 G protein-coupled |
| | receptor 1 /FL = gb: NM_005279.1 |
| 5626 | gb: NM_006200.1 /DEF = |
| | Homo sapiens proprotein convertase subtilisinkexin |
| | type 5 (PCSK5), mRNA. /FEA = mRNA /GEN = |
| | PCSK5 /PROD = proprotein convertase subtilisinkexin type 5 |
| | /DB_XREF = gi: 11321618 /UG = Hs.94376 |
| | proprotein convertase subtilisinkexin type 5 /FL = gb: |
| | NM_006200.1 gb: U56387.2 |

Gene 11541 shows no correlation with PSA in G3/4 samples, whereas gene 14523 shows correlation in BPH samples. Thus, 11541 is possibly the result of some overfitting due to the fact that pre-operative PSAs are available for only 7 BPH samples. Gene 14523 appears to be the most correlated gene with PSA in all samples. Gene 5626, also listed in Table 35, has good correlation coefficients (R_{BPH}²=0.44, R_{G34}²=0.58).

Reports are found in the published literature indicating that G Protein-coupled receptors such as gene 14523 are important in characterizing prostate cancer. See, e.g. L. L. Xu, et al. Cancer Research 60, 6568-6572, Dec. 1, 2000.

For comparison, genes that have “prostate specific antigen” in their description (none had PSA) were considered:
Gene 4649: gb:NM_{—}001648.1/DEF=Homo sapiens kallikrein 3, (prostate specific antigen) (KLK3), mRNA./FEA=mRNA/GEN=KLK3/PROD=kallikrein 3, (prostate specific antigen)/DB_XREF=gi:4502172/UG=Hs. 171995 kallikrein 3, (prostate specific antigen)/FL=gb:BC005307.1 gb:NM_{—}001648.1 gb:U17040.1 gb:M26663.1; and
Gene 4650: gb:U17040.1/DEF=Human prostate specific antigen precursor mRNA, complete cds./FEA=mRNA/PROD=prostate specific antigen precursor /DB_XREF=gi:595945/UG=Hs.171995 kallikrein 3, (prostate specific antigen) /FL=gb:BC005307.1 gb:NM_{—}001648.1 gb:U17040.1 gb:M26663.1. Neither of these genes had activity that correlates with preoperative PSA.

Another test looked at finding genes whose expression correlate with cancer volume in grade 3 and 4 cancer tissues. However, even the most correlated gene is not found significant with respect to the Bonferroni-corrected pvalue (pval=0.42). Table 36 lists the top nine genes most correlated with cancer volume in G3+4 samples. The details of the top gene are provided in Table 37.

TABLE 36

| Rank | Gene ID | Unigene ID | Sign corr. | Pearson | Pval | FDR |
|---|---|---|---|---|---|---|
| 1 | 8851 | Hs.217493 | −1 | 0.6582 | 0.43 | 0.43 |
| 2 | 6892 | Hs.2868 | −1 | 0.6282 | 1 | 0.51 |
| 3 | 21353 | Hs.283803 | 1 | 0.6266 | 1 | 0.36 |
| 4 | 7731 | Hs.182507 | −1 | 0.6073 | 1 | 0.53 |
| 5 | 4853 | Hs.86958 | −1 | 0.6039 | 1 | 0.46 |
| 6 | 622 | Hs.14449 | −1 | 0.5958 | 1 | 0.48 |
| 7 | 8665 | Hs.74497 | 1 | 0.5955 | 1 | 0.41 |
| 8 | 13750 | Hs.2014 | −1 | 0.579 | 1 | 0.6 |
| 9 | 15413 | Hs.177961 | −1 | 0.5775 | 1 | 0.56 |

TABLE 37

| Gene ID | Description |
|---|---|
| 8851 | gb: M62898.1 /DEF = Human lipocortin (LIP) |
| | 2 pseudogene mRNA, complete |
| | cdslike region. /FEA = mRNA /DB_XREF = |
| | gi: 187147 /UG = Hs.217493 annexin |
| | A2 /FL = gb: M62898.1 |

A lipocortin has been described in U.S. Pat. No. 6,395,715 entitled “Uteroglobin gene therapy for epithelial cell cancer”. Using RT-PCR, under-expression of lipocortin in cancer compared to BPH has been reported by Kang J S et al., Clin Cancer Res. 2002 January; 8(1):117-23.

### Example 6

### Prostate Cancer Comparative Study of Stamey Data (12-2004)

In this example sets of genes obtained with two different data sets are compared. Both data sets were generated by Dr. Thomas A. Stamey of Stanford University, the first in 2001 using Affymetrix HuGeneFL probe arrays (“Stamey 2001”), the second in 2003 using Affymetrix U133A chip (“Stamey 2003”). After matching the genes in both arrays, a set of about 2000 common genes was used in the study. Gene selection was performed on the data of both studies independently, then the resulting gene sets were compared. A remarkable agreement was found. In addition, classifiers were trained on one dataset and tested on the other. In the separation tumor (G3/4) vs. all other tissues, classification accuracies comparable to those obtained in previous reports were obtained by cross-validation on the second study: 10% error can be achieved with 10 genes (on the independent test set of the first study); by cross-validation, there was 8% error. In the separation BPH vs. all other tissues, there was also 10% error with 10 genes. The cross-validation results for BPH were overly optimistic (only one error), however this was not unexpected since there were only 10 BPH samples in the second study. Tables of genes were selected by consensus of both studies.

The Stamey 2001 (first) data set consisted of 67 samples from 26 patients. The Affymetrix HuGeneFL probe arrays used have 7129 probes, representing ˜6500 genes. The composition of the 2001 dataset (number of samples in parenthesis) is summarized in Table 38. Several grades and zones are represented, however, all TZ samples are BPH (no cancer), all CZ samples are normal (no cancer). Only the PZ contains a variety of samples. Also, many samples came from the same tissues.

TABLE 38

| Zone | Histological classification |
|---|---|
| CZ (3) | NL (3) |
| PZ (46) | NL (5) |
| | Stroma (1) |
| | Dysplasia (3) |
| | G3 (10) |
| | G4 (27) |
| TZ (18) | BPH (18) |
| Total 67 | |

The Stamey 2003 (second) dataset consisted of a matrix of 87 lines (samples) and 22283 columns (genes) obtained from an Affymetrix U133A chip. The distribution of the samples of the microarray prostate cancer study is given as been provided previously in Table 12.

Genes that had the same Gene Accession Number (GAN) in the two arrays HuGeneFL and U133A were selected. The selection was further limited to descriptions that matched reasonably well. For that purpose, a list of common words was created. A good match corresponds to a pair of description having at least one common word, excluding these common words, short words (fewer that 3 letters) and numbers. The resulting set included 2346 genes.

Because the data from both studies had previously been normalized using different methods, it was re-normalized using the routine provided below. Essentially, the data is translated and scaled, the log is taken, the lines and columns are normalized; the outlier values are squashed. This preprocessing was selected based on a visual examination of the data.

For the 2001 study, a bias of −0.08 was used. For the 2003 study, the bias was 0. Visual examination revealed that these values stabilize the variance of both classes reasonably well.

The set of 2346 genes was ranked using the data of both studies independently, with the area under the ROC curve (AUC) being used as the ranking criterion. P values were computed with the Bonferroni correction and False discovery rate (FDR) was calculated.

Both rankings were compared by examining the correlation of the AUC scores. Cross-comparisons were done by selecting the top 50 genes in one study and examining how “enriched” in those genes were the lists of top ranking genes from the other study, varying the number of genes. This can be compared to a random ranking. For a consensus ranking, the genes were ranked according to their smallest score in the two studies.

Reciprocal tests were run in which the data from one study was used for training of the classifier which was then tested on the data from the other study. Three different classifiers were used: Linear SVM, linear ridge regression, and Golub's classifier (analogous to Naïve Bayes). For every test, the features selected with the training set were used. For comparison, the consensus features were also used.

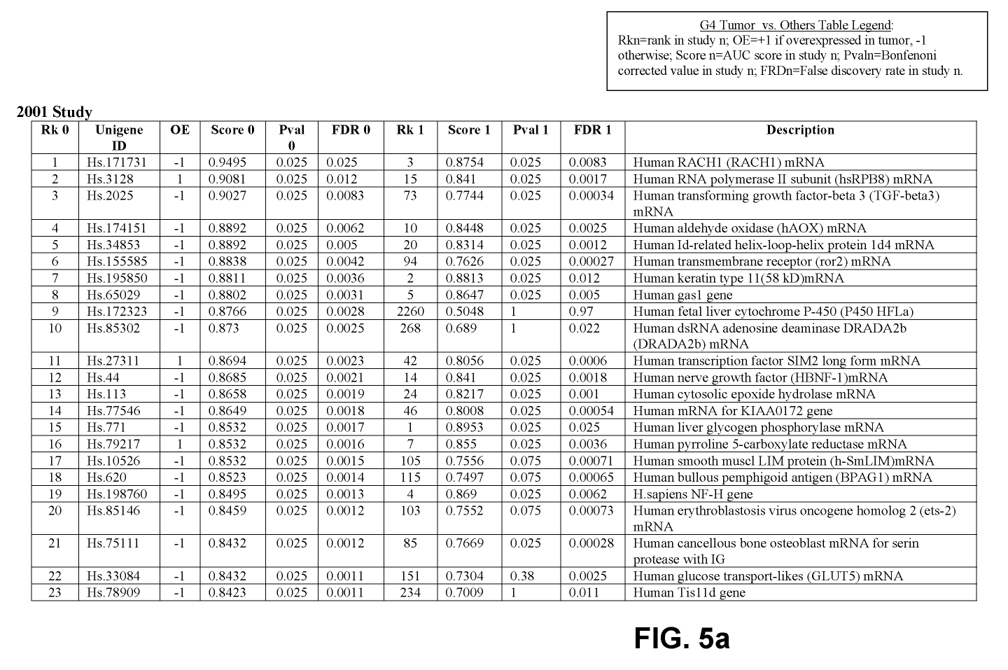

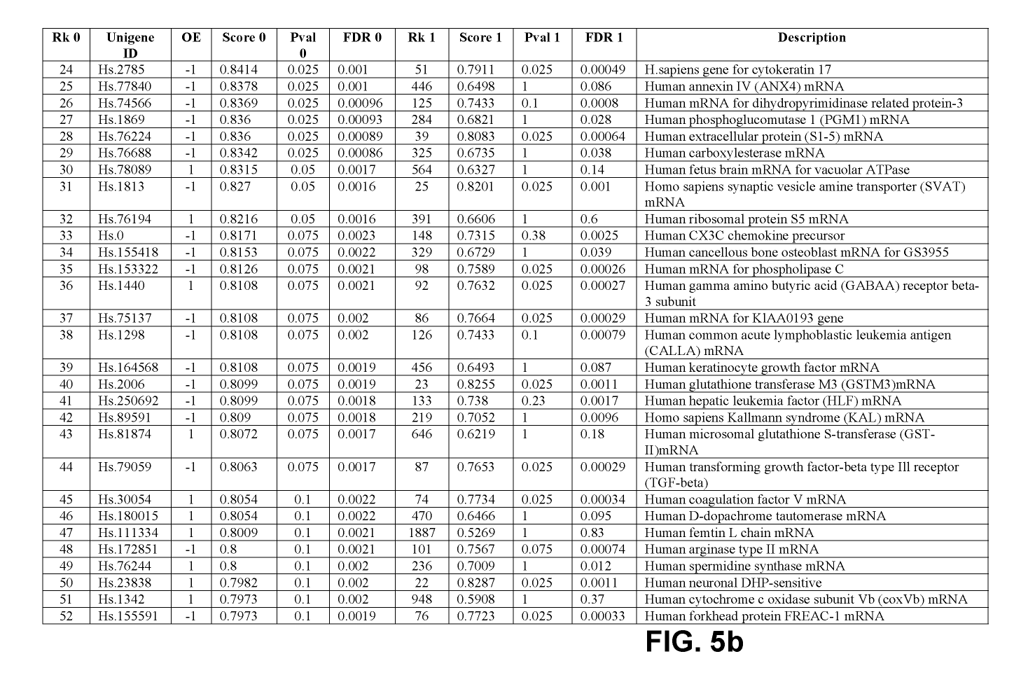

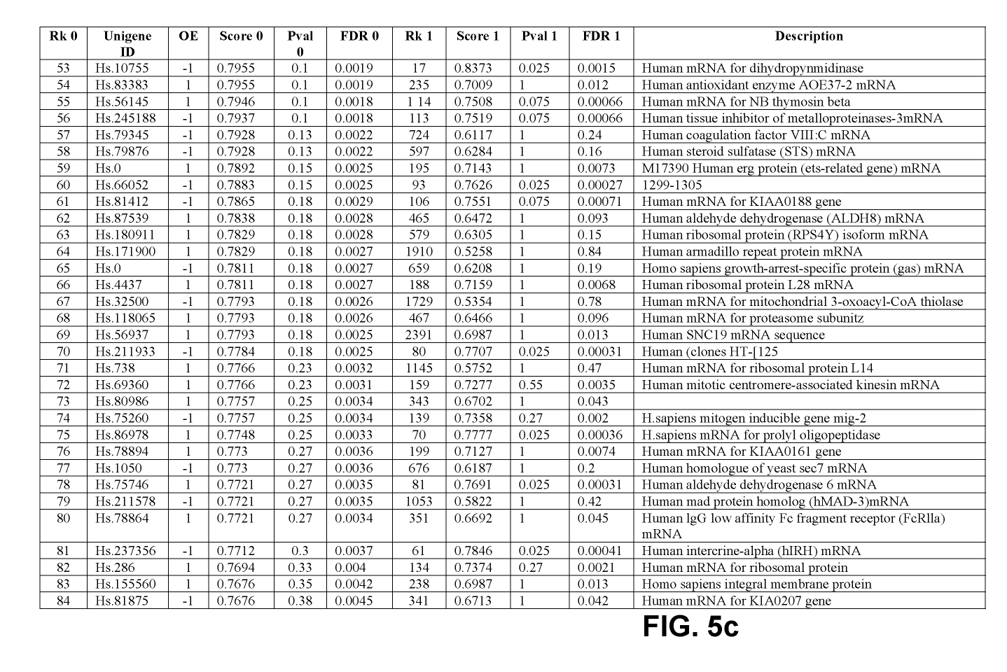

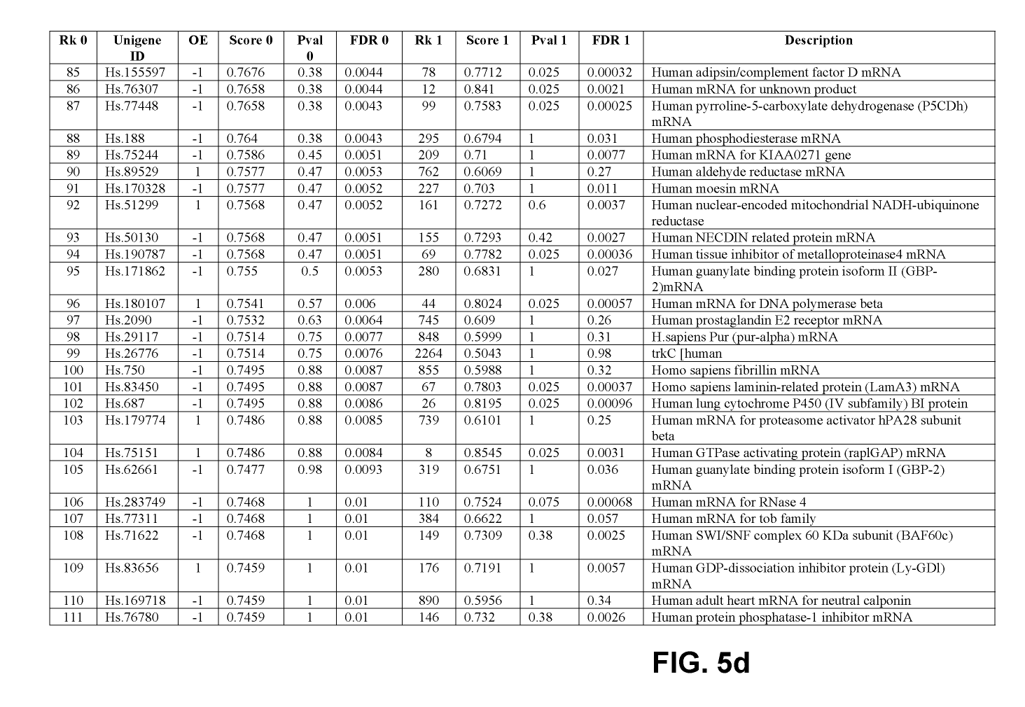

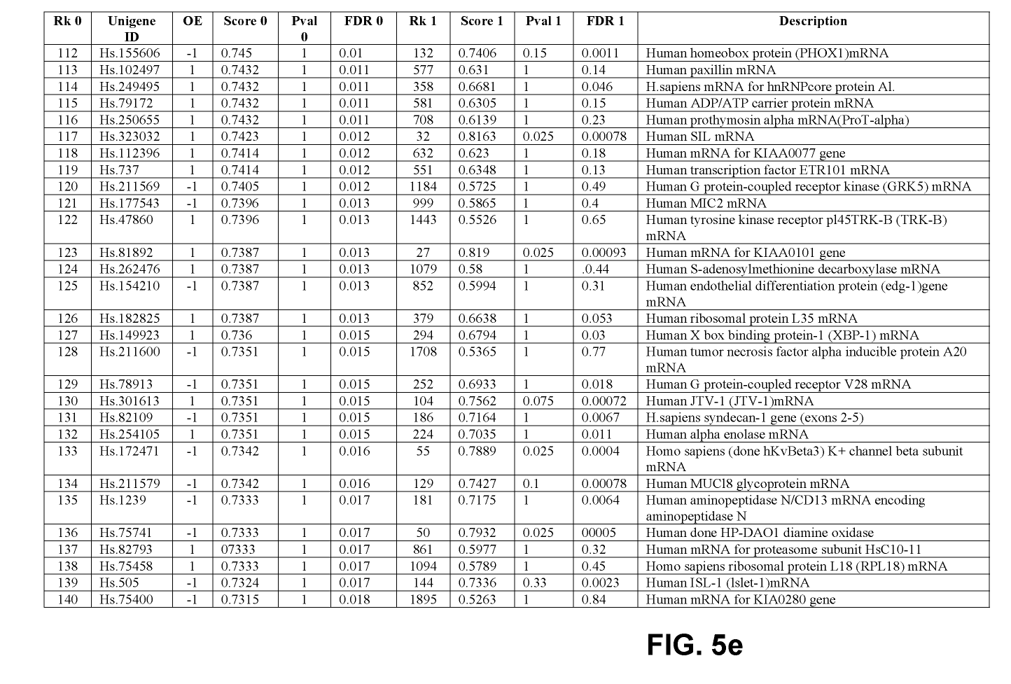

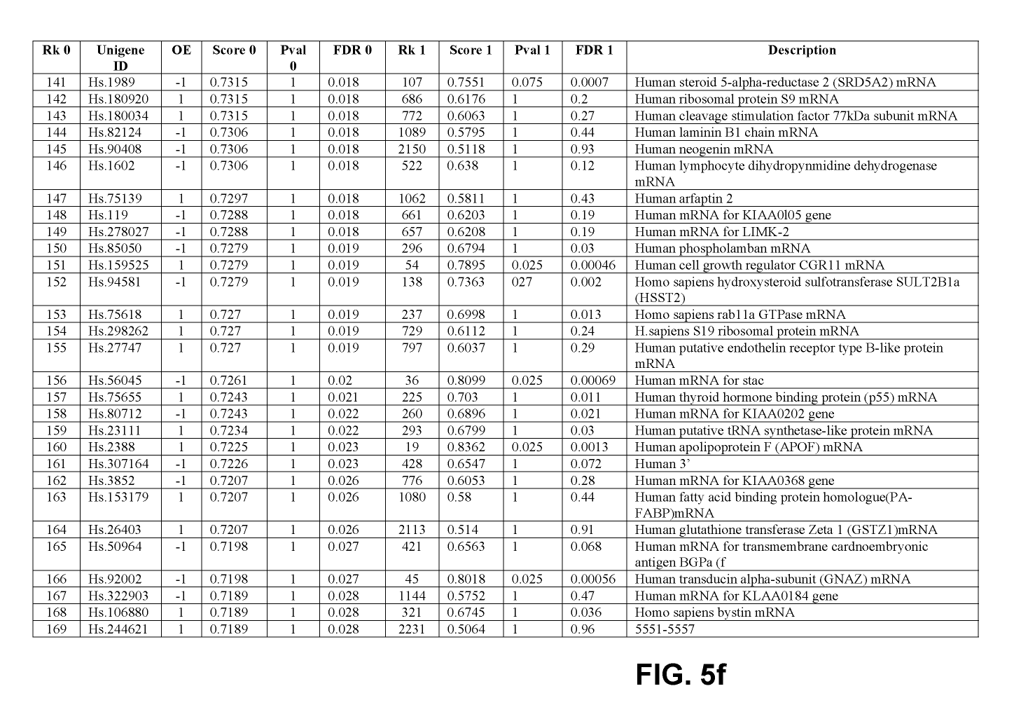

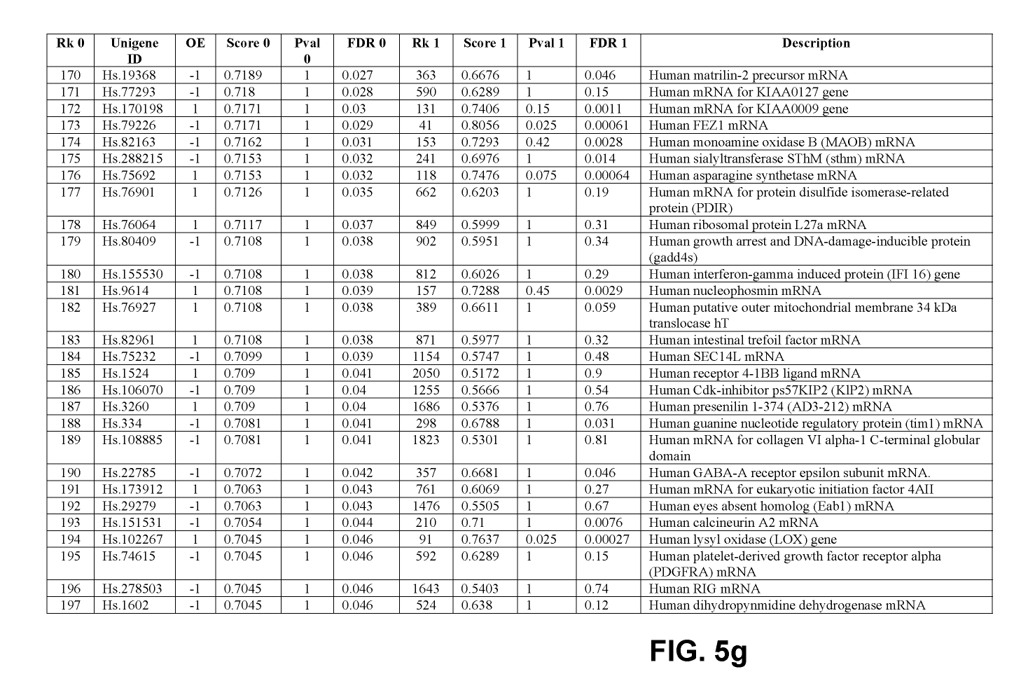

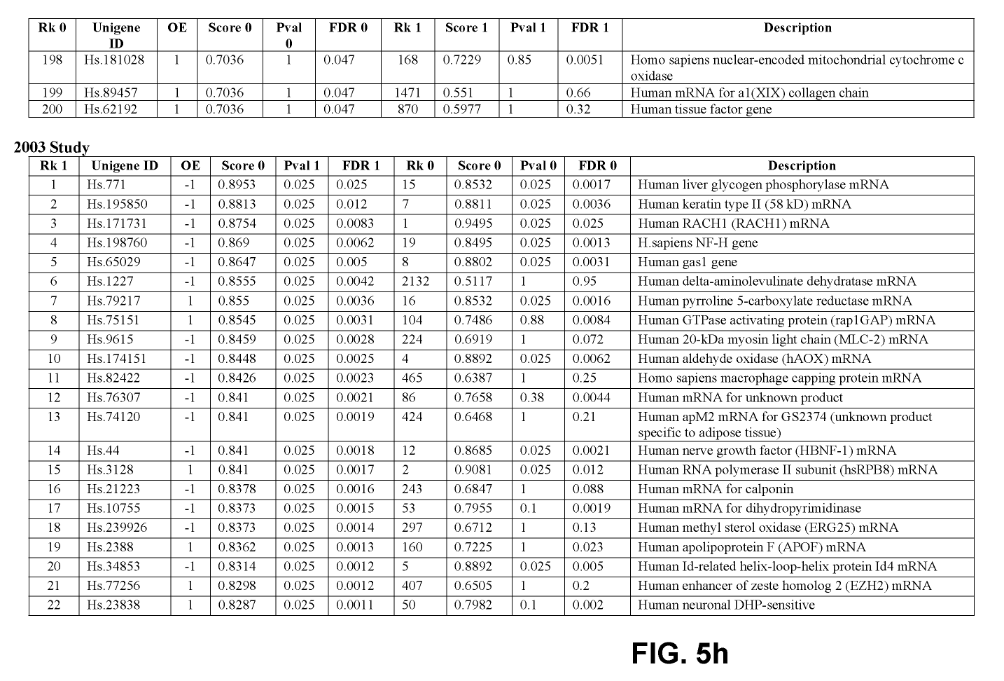

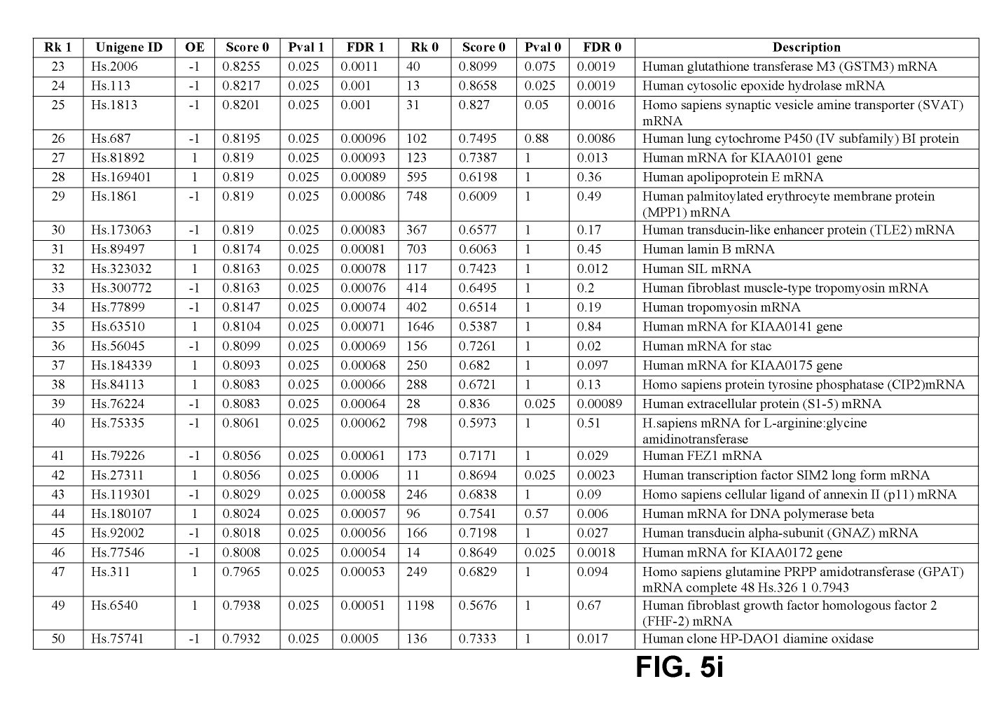

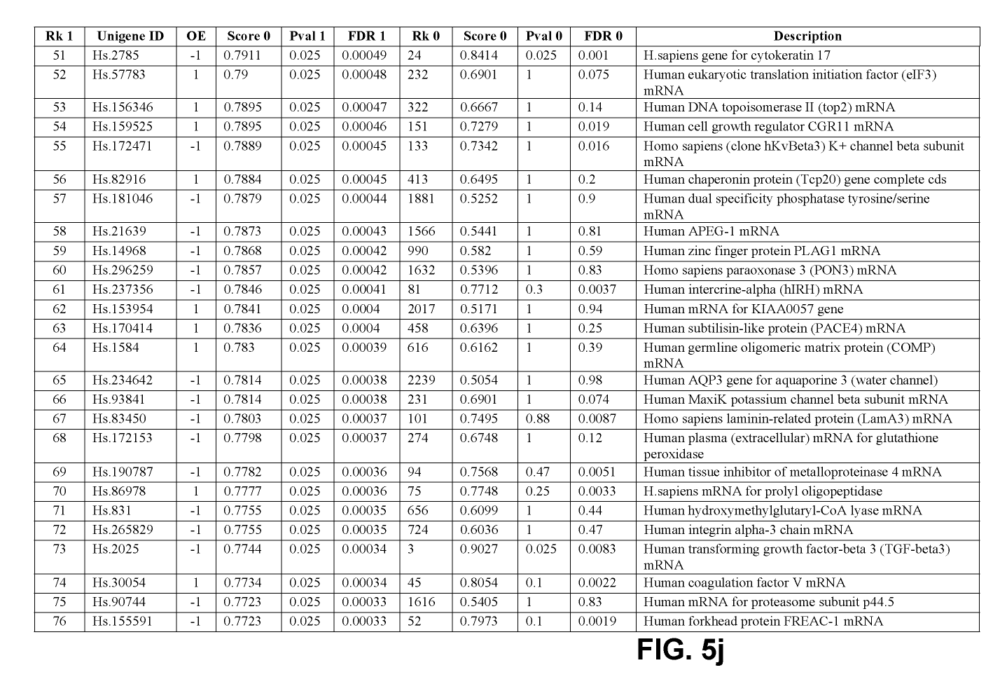

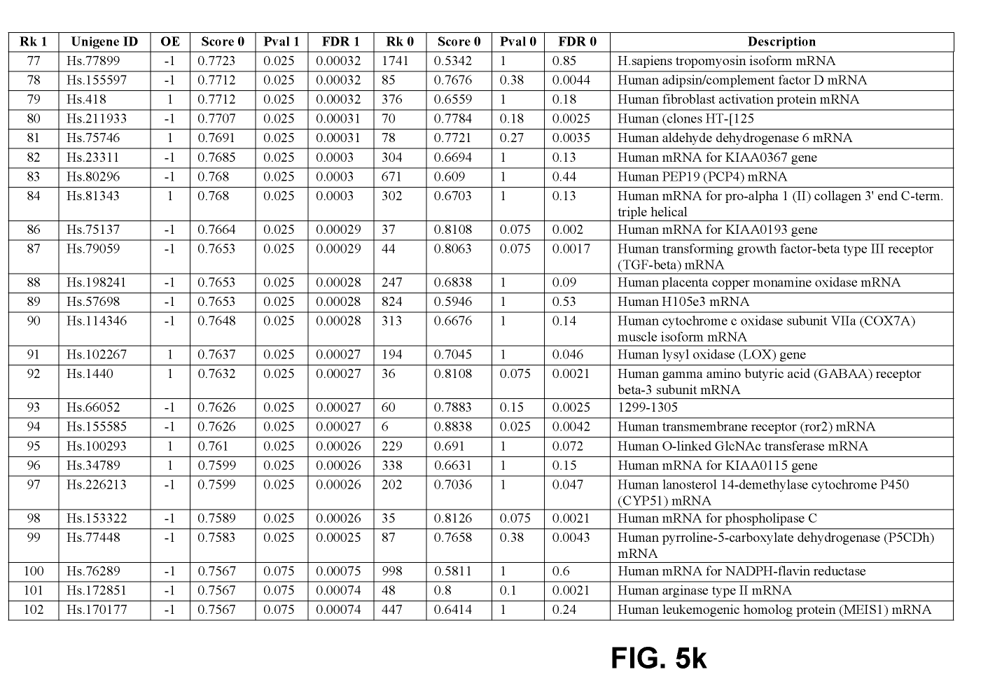

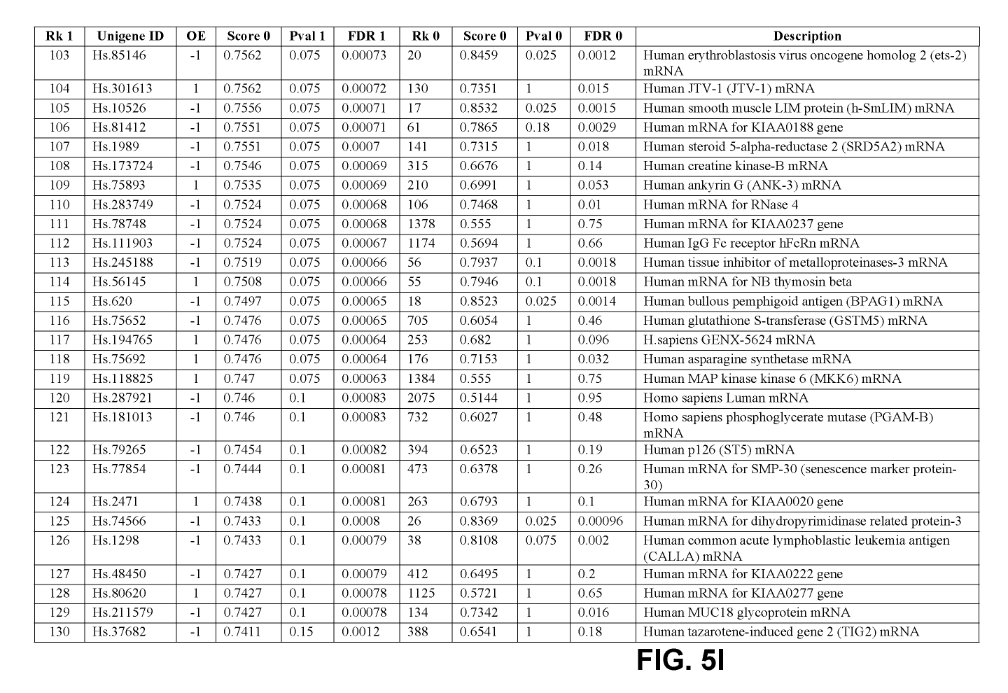

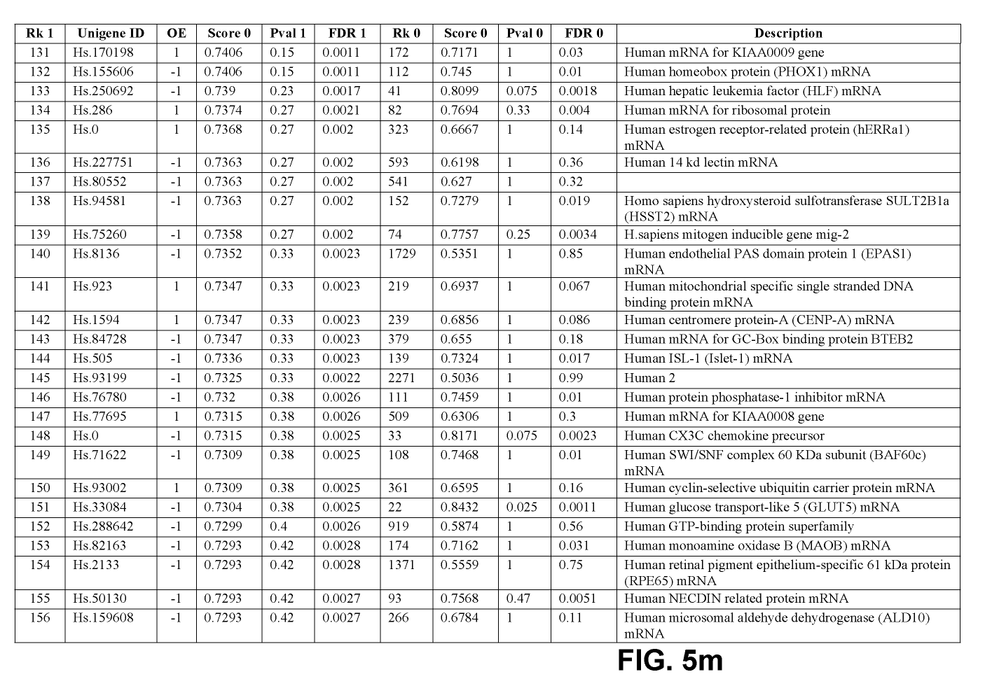

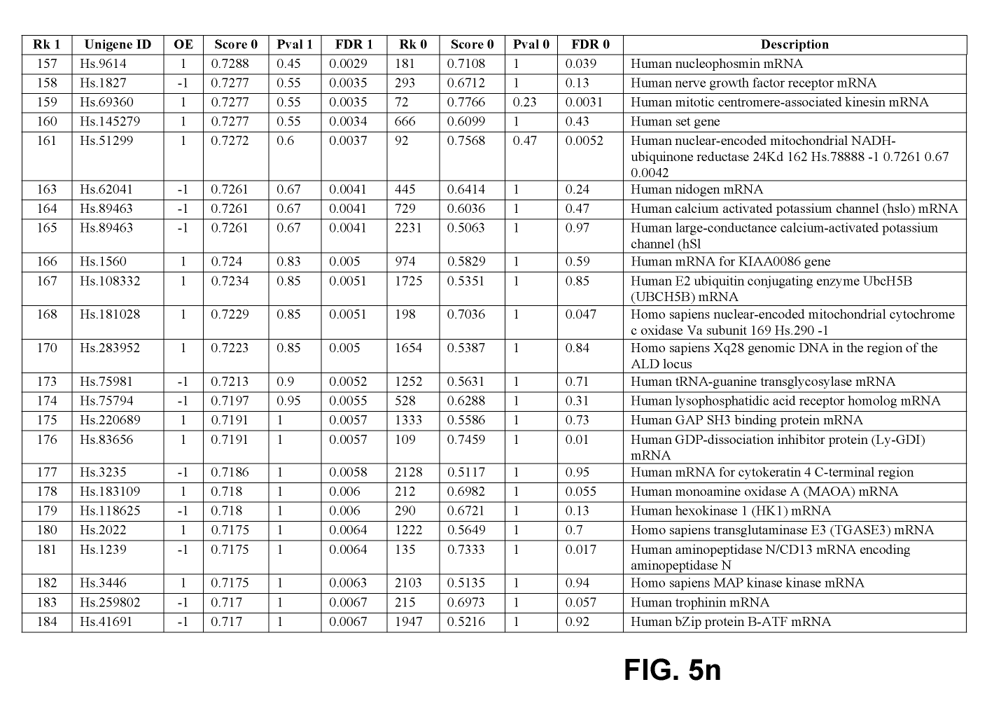

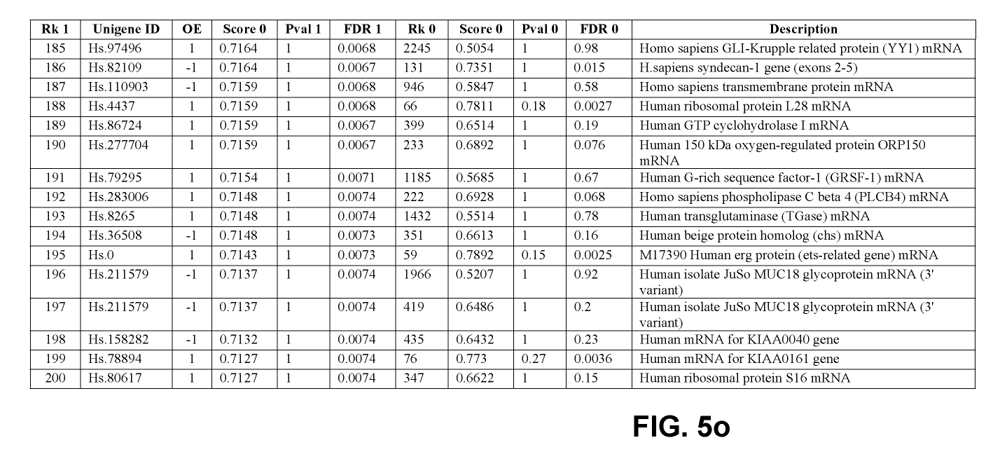

Separation of all tumor samples (G3 and G4) from all others was performed, with the G3 and G4 samples being grouped into the positive class and all samples grouped into the negative class. The top 200 genes in each study of Tumor G3/4 vs. others are listed in the tables in FIGS. 5a-5o for the 2001 study and the 2003 study. The genes were ranked in two ways, using the data of the first study (2001) and using the data of the second study (2003)

Most genes ranking high in one study also rank high in the other, with some notable exceptions. These exceptions may correspond to probes that do not match in both arrays even though their gene identification and descriptions match. They may also correspond to probes that “failed” to work in one array.

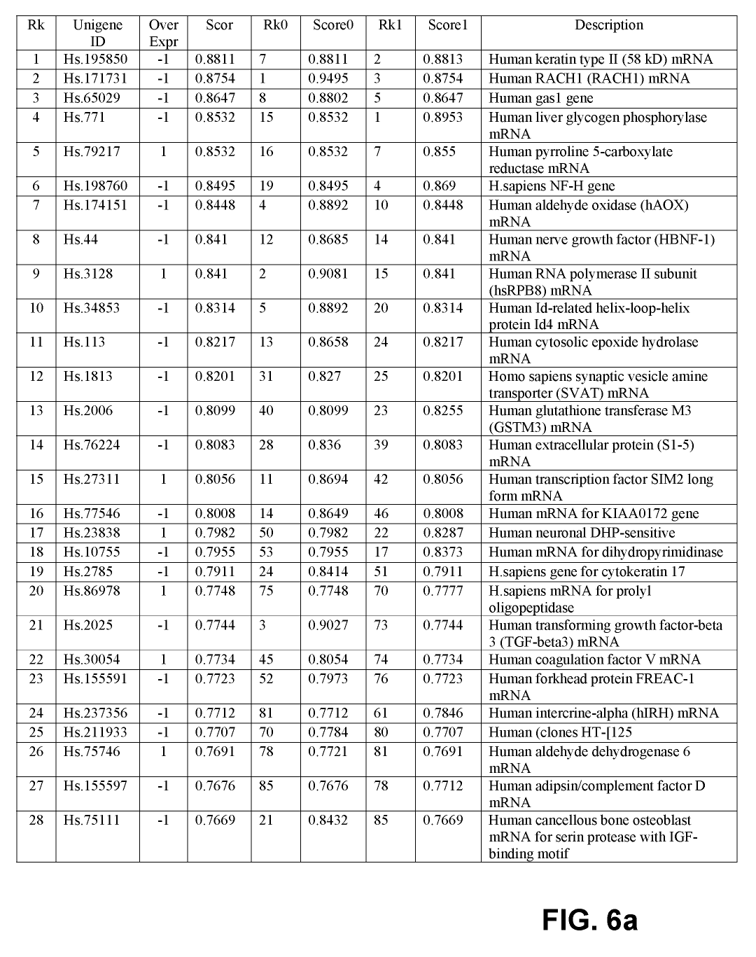

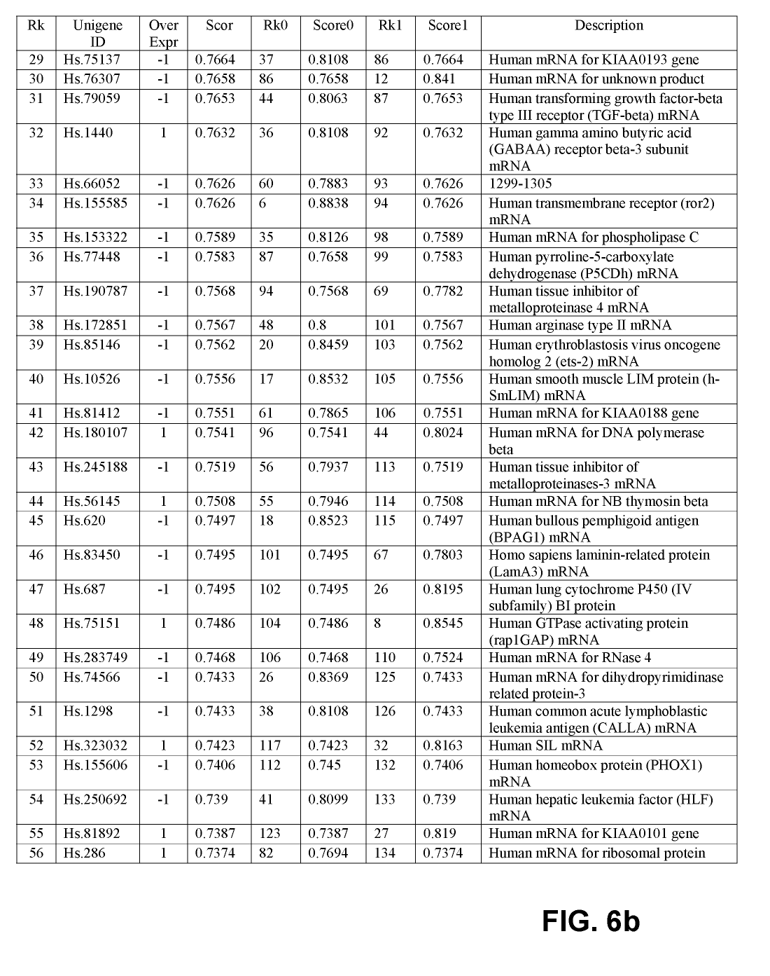

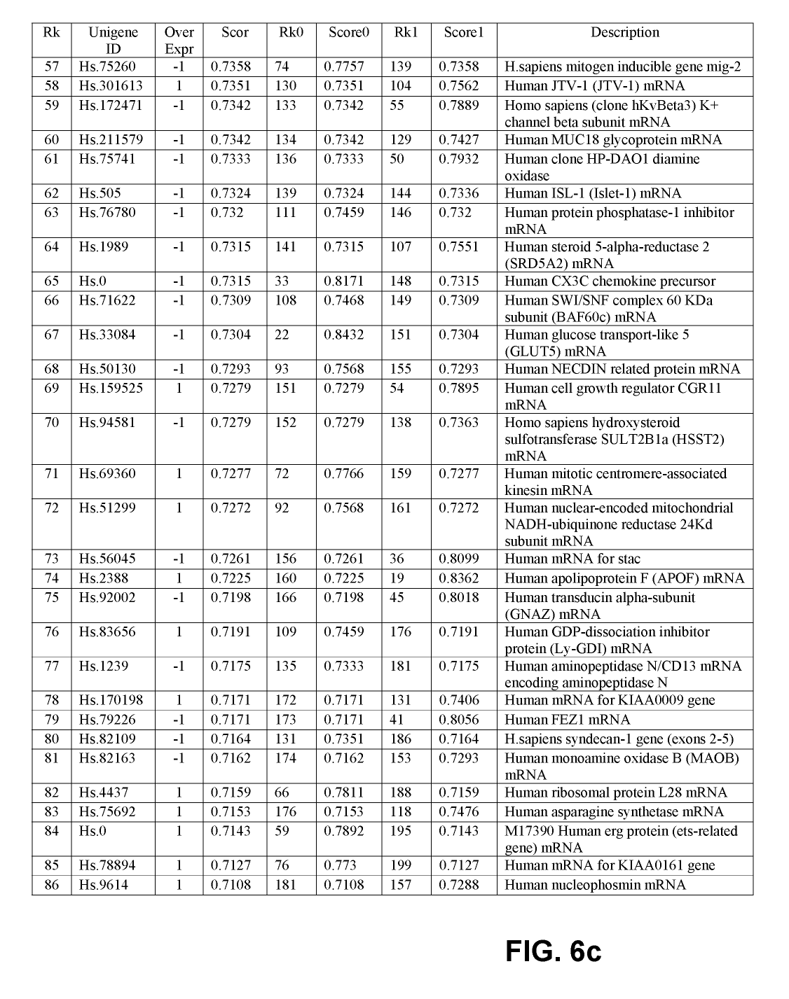

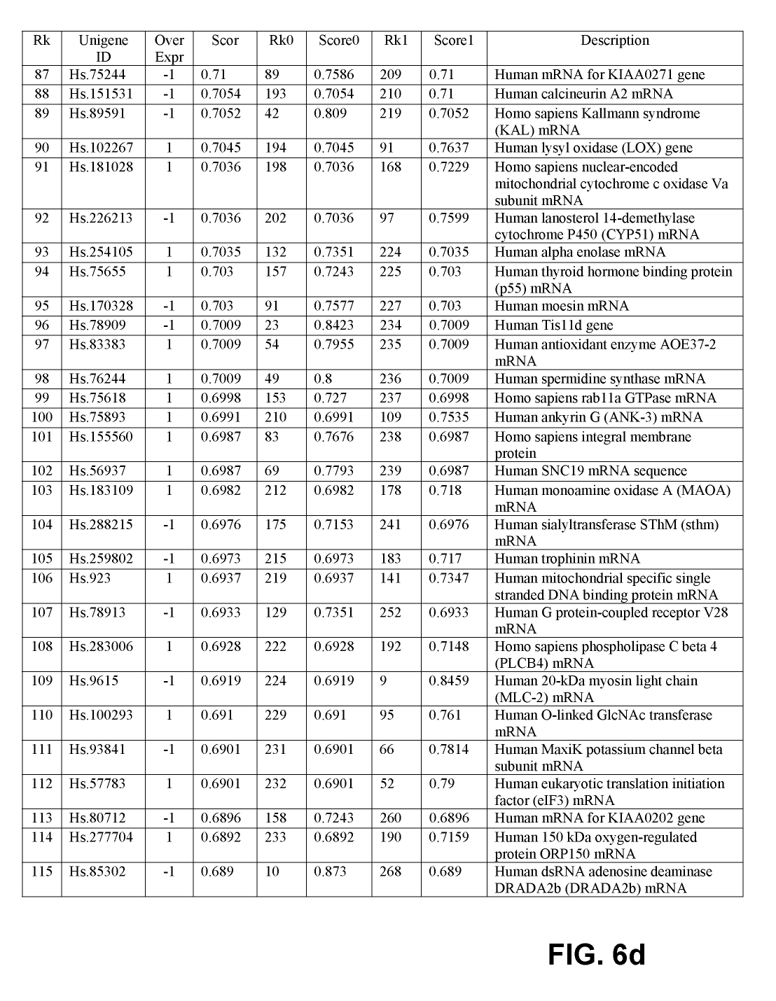

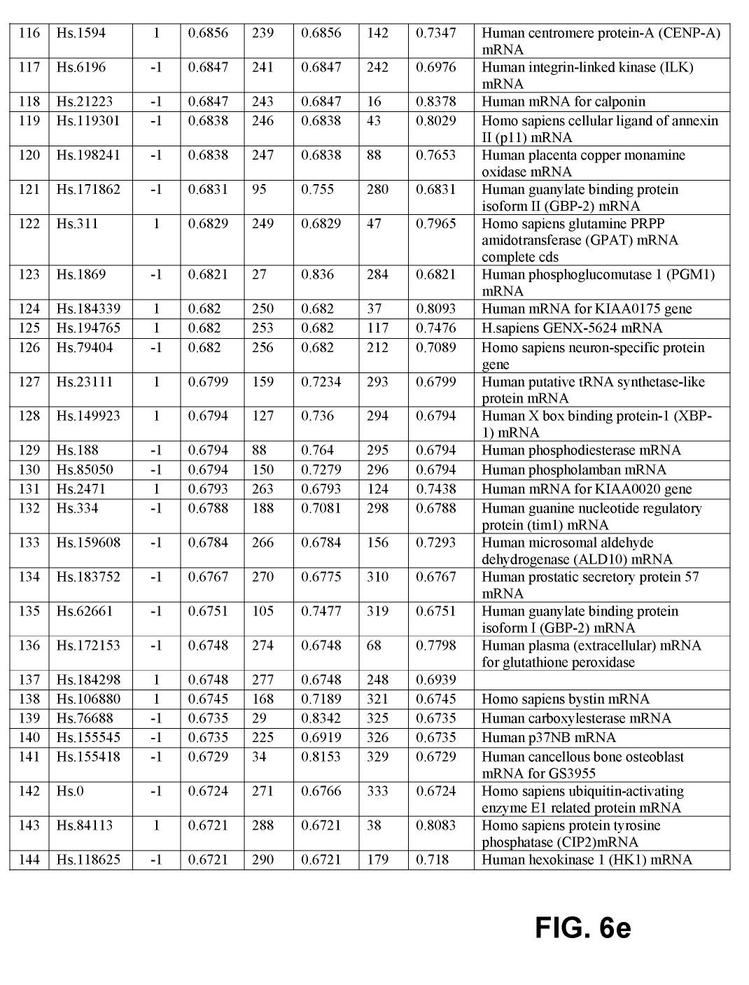

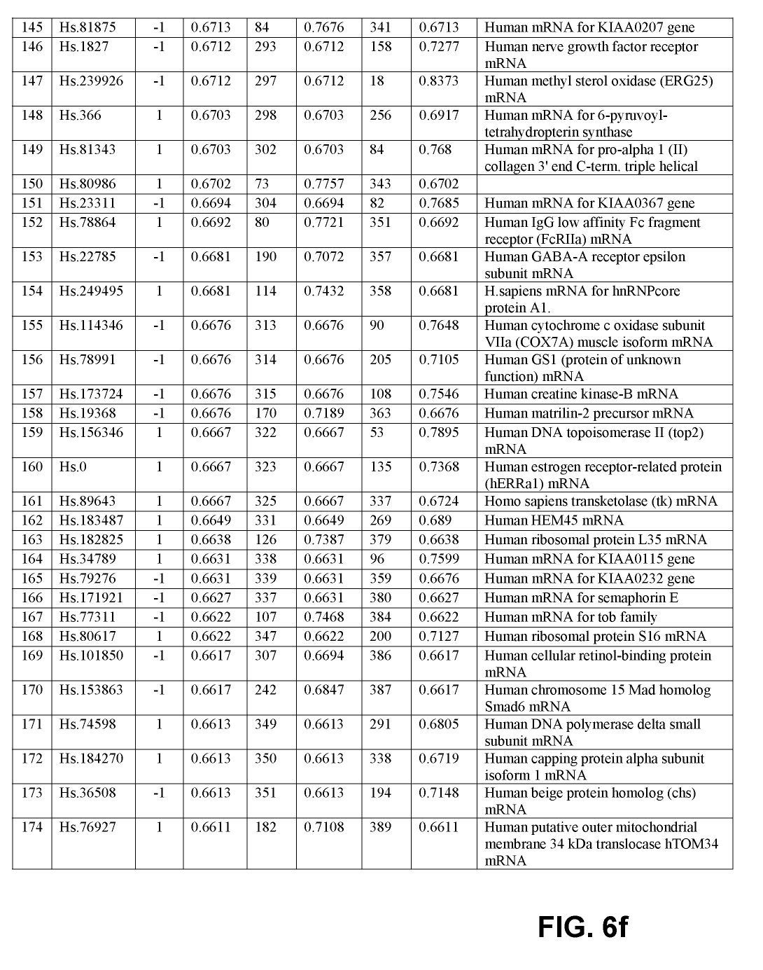

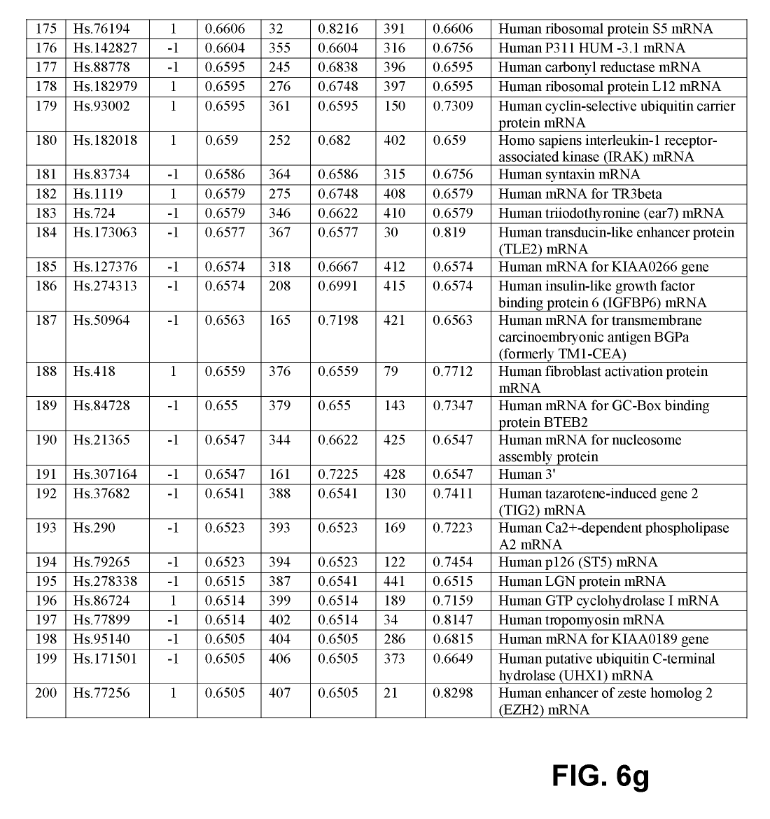

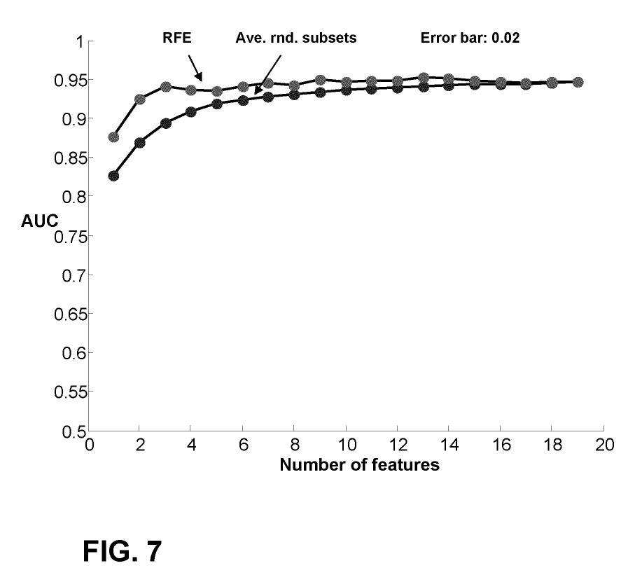

Table 39 lists the top 50 genes resulting from the feature ranking by consensus between the 2001 study and the 2003 study Tumor G3/4 vs. others. A listing of the top 200 genes, including the 50 genes in Table 39, is provided in FIGS. 6a-6g. Ranking was performed according to a score that is the minimum of score0 and score1

TABLE 39

| | Unigene | Over | | | | | | |
|---|---|---|---|---|---|---|---|---|
| Rk | ID | Expr | Scor | Rk0 | Score0 | Rk1 | Score1 | Description |
| 1 | Hs.195850 | −1 | 0.8811 | 7 | 0.8811 | 2 | 0.8813 | Human keratin type II (58 kD) |
| | | | | | | | | mRNA |
| 2 | Hs.171731 | −1 | 0.8754 | 1 | 0.9495 | 3 | 0.8754 | Human RACH1 (RACH1) mRNA |
| 3 | Hs.65029 | −1 | 0.8647 | 8 | 0.8802 | 5 | 0.8647 | Human gas1 gene |
| 4 | Hs.771 | −1 | 0.8532 | 15 | 0.8532 | 1 | 0.8953 | Human liver glycogen |
| | | | | | | | | phosphorylase mRNA |
| 5 | Hs.79217 | 1 | 0.8532 | 16 | 0.8532 | 7 | 0.855 | Human pyrroline 5-carboxylate |
| | | | | | | | | reductase mRNA |
| 6 | Hs.198760 | −1 | 0.8495 | 19 | 0.8495 | 4 | 0.869 | H. sapiens NF-H gene |
| 7 | Hs.174151 | −1 | 0.8448 | 4 | 0.8892 | 10 | 0.8448 | Human aldehyde oxidase (hAOX) |
| | | | | | | | | mRNA |
| 8 | Hs.44 | −1 | 0.841 | 12 | 0.8685 | 14 | 0.841 | Human nerve growth factor (HBNF- |
| | | | | | | | | 1) mRNA |
| 9 | Hs.3128 | 1 | 0.841 | 2 | 0.9081 | 15 | 0.841 | Human RNA polymerase II subunit |
| | | | | | | | | (hsRPB8) mRNA |
| 10 | Hs.34853 | −1 | 0.8314 | 5 | 0.8892 | 20 | 0.8314 | Human Id-related helix-loop-helix |
| | | | | | | | | protein Id4 mRNA |
| 11 | Hs.113 | −1 | 0.8217 | 13 | 0.8658 | 24 | 0.8217 | Human cytosolic epoxide hydrolase |
| | | | | | | | | mRNA |
| 12 | Hs.1813 | −1 | 0.8201 | 31 | 0.827 | 25 | 0.8201 | Homo sapiens synaptic vesicle |
| | | | | | | | | amine transporter (SVAT) mRNA |
| 13 | Hs.2006 | −1 | 0.8099 | 40 | 0.8099 | 23 | 0.8255 | Human glutathione transferase M3 |
| | | | | | | | | (GSTM3) mRNA |
| 14 | Hs.76224 | −1 | 0.8083 | 28 | 0.836 | 39 | 0.8083 | Human extracellular protein (S1-5) |
| | | | | | | | | mRNA |
| 15 | Hs.27311 | 1 | 0.8056 | 11 | 0.8694 | 42 | 0.8056 | Human transcription factor SIM2 |
| | | | | | | | | long form mRNA |
| 16 | Hs.77546 | −1 | 0.8008 | 14 | 0.8649 | 46 | 0.8008 | Human mRNA for KIAA0172 gene |
| 17 | Hs.23838 | 1 | 0.7982 | 50 | 0.7982 | 22 | 0.8287 | Human neuronal DHP-sensitive |
| 18 | Hs.10755 | −1 | 0.7955 | 53 | 0.7955 | 17 | 0.8373 | Human mRNA for |
| | | | | | | | | dihydropyrimidinase |
| 19 | Hs.2785 | −1 | 0.7911 | 24 | 0.8414 | 51 | 0.7911 | H. sapiens gene for cytokeratin 17 |
| 20 | Hs.86978 | 1 | 0.7748 | 75 | 0.7748 | 70 | 0.7777 | H. sapiens mRNA for prolyl |
| | | | | | | | | oligopeptidase |
| 21 | Hs.2025 | −1 | 0.7744 | 3 | 0.9027 | 73 | 0.7744 | Human transforming growth factor- |
| | | | | | | | | beta 3 (TGF-beta3) mRNA |
| 22 | Hs.30054 | 1 | 0.7734 | 45 | 0.8054 | 74 | 0.7734 | Human coagulation factor V mRNA |
| 23 | Hs.155591 | −1 | 0.7723 | 52 | 0.7973 | 76 | 0.7723 | Human forkhead protein FREAC-1 |
| | | | | | | | | mRNA |
| 24 | Hs.237356 | −1 | 0.7712 | 81 | 0.7712 | 61 | 0.7846 | Human intercrine-alpha (hIRH) |
| | | | | | | | | mRNA |
| 25 | Hs.211933 | −1 | 0.7707 | 70 | 0.7784 | 80 | 0.7707 | Human (clones HT-[125 |
| 26 | Hs.75746 | 1 | 0.7691 | 78 | 0.7721 | 81 | 0.7691 | Human aldehyde dehydrogenase 6 |
| | | | | | | | | mRNA |
| 27 | Hs.155597 | −1 | 0.7676 | 85 | 0.7676 | 78 | 0.7712 | Human adipsin/complement factor D |
| | | | | | | | | mRNA |
| 28 | Hs.75111 | −1 | 0.7669 | 21 | 0.8432 | 85 | 0.7669 | Human cancellous bone osteoblast |
| | | | | | | | | mRNA for serin protease with IGF- |
| | | | | | | | | binding motif |
| 29 | Hs.75137 | −1 | 0.7664 | 37 | 0.8108 | 86 | 0.7664 | Human mRNA for KIAA0193 gene |
| 30 | Hs.76307 | −1 | 0.7658 | 86 | 0.7658 | 12 | 0.841 | Human mRNA for unknown product |
| 31 | Hs.79059 | −1 | 0.7653 | 44 | 0.8063 | 87 | 0.7653 | Human transforming growth factor- |
| | | | | | | | | beta type III receptor (TGF-beta) |
| | | | | | | | | mRNA |
| 32 | Hs.1440 | 1 | 0.7632 | 36 | 0.8108 | 92 | 0.7632 | Human gamma amino butyric acid |
| | | | | | | | | (GABAA) receptor beta-3 subunit |
| | | | | | | | | mRNA |
| 33 | Hs.66052 | −1 | 0.7626 | 60 | 0.7883 | 93 | 0.7626 | 1299-1305 |
| 34 | Hs.155585 | −1 | 0.7626 | 6 | 0.8838 | 94 | 0.7626 | Human transmembrane receptor |
| | | | | | | | | (ror2) mRNA |
| 35 | Hs.153322 | −1 | 0.7589 | 35 | 0.8126 | 98 | 0.7589 | Human mRNA for phospholipase C |
| 36 | Hs.77448 | −1 | 0.7583 | 87 | 0.7658 | 99 | 0.7583 | Human pyrroline-5-carboxylate |
| | | | | | | | | dehydrogenase (P5CDh) mRNA |
| 37 | Hs.190787 | −1 | 0.7568 | 94 | 0.7568 | 69 | 0.7782 | Human tissue inhibitor of |
| | | | | | | | | metalloproteinase 4 mRNA |
| 38 | Hs.172851 | −1 | 0.7567 | 48 | 0.8 | 101 | 0.7567 | Human arginase type II mRNA |
| 39 | Hs.85146 | −1 | 0.7562 | 20 | 0.8459 | 103 | 0.7562 | Human erythroblastosis virus |
| | | | | | | | | oncogene homolog 2 (ets-2) mRNA |
| 40 | Hs.10526 | −1 | 0.7556 | 17 | 0.8532 | 105 | 0.7556 | Human smooth muscle LIM protein |
| | | | | | | | | (h-SmLIM) mRNA |
| 41 | Hs.81412 | −1 | 0.7551 | 61 | 0.7865 | 106 | 0.7551 | Human mRNA for KIAA0188 gene |
| 42 | Hs.180107 | 1 | 0.7541 | 96 | 0.7541 | 44 | 0.8024 | Human mRNA for DNA polymerase |
| | | | | | | | | beta |
| 43 | Hs.245188 | −1 | 0.7519 | 56 | 0.7937 | 113 | 0.7519 | Human tissue inhibitor of |
| | | | | | | | | metalloproteinases-3 mRNA |
| 44 | Hs.56145 | 1 | 0.7508 | 55 | 0.7946 | 114 | 0.7508 | Human mRNA for NB thymosin |
| | | | | | | | | beta |
| 45 | Hs.620 | −1 | 0.7497 | 18 | 0.8523 | 115 | 0.7497 | Human bullous pemphigoid antigen |
| | | | | | | | | (BPAG1) mRNA |
| 46 | Hs.83450 | −1 | 0.7495 | 101 | 0.7495 | 67 | 0.7803 | Homo sapiens laminin-related |
| | | | | | | | | protein (LamA3) mRNA |
| 47 | Hs.687 | −1 | 0.7495 | 102 | 0.7495 | 26 | 0.8195 | Human lung cytochrome P450 (IV |
| | | | | | | | | subfamily) BI protein |
| 48 | Hs.75151 | 1 | 0.7486 | 104 | 0.7486 | 8 | 0.8545 | Human GTPase activating protein |
| | | | | | | | | (rap1GAP) mRNA |
| 49 | Hs.283749 | −1 | 0.7468 | 106 | 0.7468 | 110 | 0.7524 | Human mRNA for RNase 4 |
| 50 | Hs.74566 | −1 | 0.7433 | 26 | 0.8369 | 125 | 0.7433 | Human mRNA for dihydro- |
| | | | | | | | | pyrimidinase related protein-3 |

Training of the classifier was done with the data of one study while testing used the data of the other study. The results are similar for the three classifiers that were tried: SVM, linear ridge regression and Golub classifier. Approximately 90% accuracy can be achieved in both cases with about 10 features. Better “cheating” results are obtained with the consensus features. This serves to validate the consensus features, but the performances cannot be used to predict the accuracy of a classifier on new data. An SVM was trained using the two best features of the 2001 study and the sample of the 2001 study as the training data. The samples from the 2003 study were used as test data to achieve an error rate of 16% is achieved. The tumor and non-tumor samples are well separated, but that, in spite of normalization, the distributions of the samples is different between the two studies.

The definitions of the statistics used in the various rankings are provided in Table 40.

TABLE 40

| Statistic | Description |
|---|---|
| AUC | Area under the ROC curve of individual genes, using training tissues. The ROC curve |
| | (receiver operating characteristic) is a plot of the sensitivity (error rate of the “positive” |
| | class) vs. the specificity (error rate of the “negative” class). Insignificant genes have an |
| | AUC close to 0.5. Genes with an AUC closer to one are overexpressed in cancer. Genes |
| | with an AUC closer to zero are underexpressed. |
| pval | Pvalue of the AUC, used as a test statistic to test the equality of the median of the two |
| | population (cancer and non-cancer.) The AUC is the Mann-Withney statistic. The test is |
| | equivalent to the Wilcoxon rank sum test. Small pvalues shed doubt on the null |
| | hypothesis of equality of the medians. Hence smaller values are better. To account to the |
| | multiple testing the pvalue may be Bonferroni corrected by multiplying it by the number |
| | of genes 7129. |
| FDR | False discovery rate of the AUC ranking. An estimate of the fraction of insignificant |
| | genes in the genes ranking higher than a given gene. It is equal the pvalue multiplied by |
| | the number of genes 7129 and divided by the rank. |
| Fisher | Fisher statistic characterizing the multiclass discriminative power for the histological |
| | classes (normal, BPH, dysplasia, grade 3, and grade 4.) The Fisher statistic is the ratio of |
| | the between-class variance to the within-class variance. Higher values indicate better |
| | discriminative power. The Fisher statistic can be interpreted as a signal to noise ratio. It |
| | is computed with training data only. |
| Pearson | Pearson correlation coefficient characterizing “disease progression”, with histological |
| | classes coded as 0 = normal, 1 = BPH, 2 = dysplasia, 3 = grade 3, and 4 = grade 4.) A value |
| | close to 1 indicates a good correlation with disease progression. |
| FC | Fold change computed as the ratio of the average cancer expression values to the |
| | avarage of the other expression values. It is computed with training data only. A value |
| | near one indicates an insignificant gene. A large value indicates a gene overexpressed in |
| | cancer; a small value an underexpressed gene. |
| Mag | Gene magnitude. The average of the largest class expression value (cancer or other) |
| | relative to that of the ACTB housekeeping gene. It is computed with training data only. |
| tAUC | AUC of the genes matched by probe and or description in the test set. It is computed |
| | with test data only, hence not all genes have a tAUC. |

### Example 7

### Genes Overexpressed in Prostate Cancer

Because they may be more readily detected using common analytical techniques, e.g., microarrays, and therefore, make better biomarker candidates for separating tumor from normal, genes that are overexpressed in prostate cancer were the focus of this analysis. RFE-SVM was performed, with training using the Stamey 2003 data (Table 12) and testing using a dataset created by merging five publicly available datasets containing prostate cancer samples processed with an Affymetrix chip (chip U95A). The merged public datasets produced a set of 164 samples (102 tumor and 62 normal), which will be referred to as the “public data” or “public dataset”. The probes in the U95A (˜12,000 probes) chip were matched with those of the U133A chip used in the 87 sample, 2003 Stamey study (28 tumor, 49 normal, 22000 probes) to obtain approximately 7,000 common probes.

To form the public dataset, several datasets were downloaded from the Internet (Table 41 and Table 42). The Oncomine website, on the Worldwide Web at oncomine.org, is a valuable resource to identify datasets, but the original data was downloaded from the author's websites. Table 41 lists prostate cancer datasets and Table 42 is multi-study or normal samples.

TABLE 41

| Name | Chip | Samples | Genes | Ref. | Comment |
|---|---|---|---|---|---|
| Febbo | U95A v2 | 52 tumor 50 normal | ~12600 | [1] | Have data. |
| Dhana | cDNA | Misc ~40 | 10000 | [2] | Difficult to |
| | | | | | understand and read |
| | | | | | data. |
| LaTulippe | U95A | 3 NL, 23 localized and 9 | ~12600 | [3] | Have data. |
| | | metastatic | | | |
| LuoJH | Hu35k | 15 tumor, 15 normal | ~9000 | [4] | Have data. Some |
| | | | | | work to understand it. |
| McGee | Hu6800 | 8 primary, 3 metastasic and 4 | 6800 | [5] | Not worth it. |
| | Ge | nonmalignant | | | |
| Welsh | U95A | 9 normal, 24 localized and 1 | ~12000 | [6] | Looks OK. |
| | | metastatic, and 21 cell lines | | | |
| LuoJ | cDNA | 16 tumor 9 BPH | ~6500 | [7] | Probably not worth it. |

TABLE 42

| Name | Chip | Samples | Genes | Ref. | Comment |
|---|---|---|---|---|---|
| Rama | Hu6800 | 343 primary and 12 | ~16000 | [8] | Looks interesting. |
| | Hu35kSubA | metastatic; include a few | | | Complex data. |
| | | prostate | | | |
| Hsiao | HuGenFL | 59 normal | ~10000 | [9] | Looks good. Same |
| | | | | | chips as Stamey 2001. |
| Su | U95a | 175 tumors, of which 24 | ~12600 | [10] | Looks good. |
| | | prostate | | | |

The datasets of Febbo, LaTulippe, Welsh, and Su are formatted as described below because they correspond to a large gene set from the same Affymetrix chip U95A.

### Febbo Dataset

File used:
Prostate_TN_final0701 allmeanScale.res
A data matrix of 102 lines (52 tumors, 50 normal) and 12600 columns was generated.
All samples are tumor or normal. No clinical data is available.

### LaTulippe Dataset—

The data was merged from individual text files (e.g. MET1_U95Av2.txt), yielding to a data matrix of 35 lines (3 normal, 23 localized, 9 metastatic) and 12626 columns. Good clinical data is available.

### Welsh Dataset

The data was read from file:
GNF prostate_data_CR61_{—}5974.xls
A matrix of 55 lines (9 normal, 27 tumor, 19 cell lines) and 12626 lines was generated. Limited clinical data is available. Some inconsistencies in tissue labeling between files.

### Su Dataset

The data was read from: classification_data.txt
A matrix of 174 lines (174 tumors of which 24 prostate) and 12533 lines was obtained. No clinical data available.
The initial analysis revealed that the Su and Welsh data were identical, so the Su dataset was removed.

TABLE 43

| | | | | | Stamey |
|---|---|---|---|---|---|
| | Febbo | LaTulippe | Welsh | Su | 2003 |
| Febbo | 12600 | 12600 | 12600 | 12533 | 312 |
| LaTulippe | 12600 | 12626 | 12626 | 12533 | 312 |
| Welsh | 12600 | 12626 | 12626 | 12533 | 312 |
| Su | 12533 | 12533 | 12533 | 12533 | 271 |
| Stamey | 312 | 312 | 312 | 271 | 22283 |

From Table 43 it is apparent that the four selected datasets used the same microarray (Affymetrix U95A GeneChip®). The Stamey 2003 data, however, used a different microarray (Affymetrix U133A GeneChip®), so only those genes common to both sets were selected. Affymetrix has published a reference on its web site (Affymetrix.com) that provides the correspondence between probes of different chip sets based upon their sequences.

Unigene IDs were used to identify 7350 corresponding probes on the two different chips. Using the best match from Affymetrix, 9512 probes were found to correspond, however, a number of these probes did not have Unigene IDs, or had mismatched Unigene IDs. Of the matched probes using both comparisons, 6839 have the same Unigene IDs. This latter set of 6839 probes was used.

The final characteristics of publicly available data are summarized in Table 44. Each dataset from the public data was preprocessed individually using the script my_normalize, provided below. A bias of zero was used for all normalizations.

function X=my_normalize(X, bias)

if nargin<2, bias=0; end

mini=min(min(X));

maxi=max(max(X));

X=(X-mini)/(maxi-mini)+bias;

idx=find(X<=0);

X(idx)=Inf;

epsi=min(min(X));

X(idx)=epsi;

X=log(X);

X=med_normalize(X);

X=med_normalize(X′)′;

X=med_normalize(X);

X=med_normalize(X′)′;

X=tan h(0.1*X);

function X=med_normalize(X)

mu=mean(X,2);

One=ones(size(X,2), 1);

XM=X-mu(:,One);

S=median(abs(XM),2);

X=XM./S(:,One);

The public data was then merged and the feature set was reduced to n. The Stamey data was normalized with my_normalize script (above) after this reduction of feature set. The public data was re-normalized with my_normalize script after this reduction of feature set.

TABLE 44

| | Histological | Number |
|---|---|---|
| Data source | classification | of samples |
| Febbo | Normal | 50 |
| | Tumor | 52 |
| LaTulippe | Normal | 3 |
| | Tumor | 23 |
| Welsh | Normal | 9 |
| | Tumor | 27 |
| Total | | 164 |

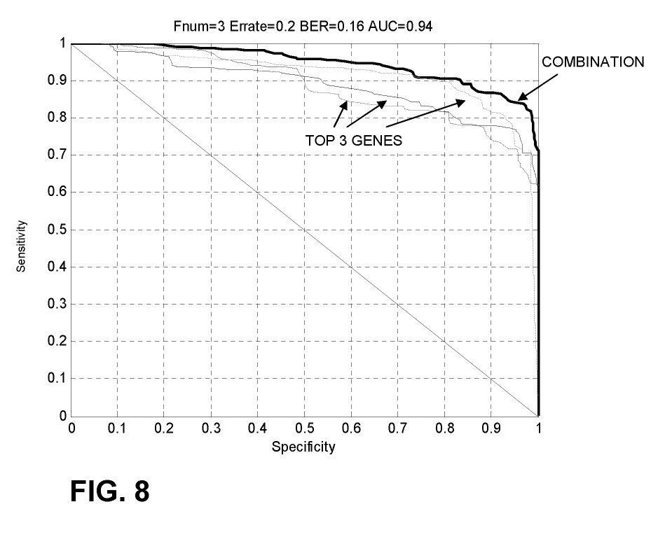

The 19 top ranking genes that were identified by RFE-SVM are listed in Tables 45a and 45b. Table 45a provides the analysis results corresponding to the original UniGene number, Affymetrix probe ID, gene symbol and description. Table 45b associates the SEQ ID NO. with the original (archival) UniGene number, the current UniGene number, and the more detailed “target description” obtained from the Affymetric GeneChip® annotation spreadsheet under the column under the same title. (The Affymetrix annotation spreadsheets for the U95 and U133 are publicly available on the World Wide Web at Affymetrix.com, and are incorporated herein by reference.) The analysis revealed that on average any combination of 4 genes or more from the 19 top ranked genes yielded an area under the curve (AUC) of 0.9 on test data. The top ranking combination of three genes as determined by RFE-SVM yielded AUC=0.94. This combination consisted of genes are AgX-1/UAP1 (Hs.21293), DKFZp564 (Hs.7780), and IMPDH2 (Hs.75432). While each of these genes performs well individually, their combination outperforms any individual gene (FIG. 8).

TABLE 45a

| UniGene | AUC | pval | FDR | Fisher | Pearson | FC | Mag | tAUC | Affy probe | Symbol/Description |
|---|---|---|---|---|---|---|---|---|---|---|
| Hs.7780 | 0.9135 | 4.50E−11 | 2.00E−07 | 29.91 | 0.69 | 2.16 | 0.077 | 0.9037 | 212412_at | DKFZp564 (PDLIM5)/ |
| | | | | | | | | | | Homo sapiens mRNA; |
| | | | | | | | | | | cDNA DKFZp564A072 |
| | | | | | | | | | | (from clone |
| | | | | | | | | | | DKFZp564A072) |
| Hs.21293 | 0.8888 | 6.00E−10 | 7.40E−07 | 19.3 | 0.69 | 2.31 | 0.0012 | 0.877 | 209340_at | UAP1/AGX-1/Homo |
| | | | | | | | | | | sapiens AgX-1 antigen |
| | | | | | | | | | | mRNA |
| Hs.79037 | 0.8829 | 1.10E−09 | 1.00E−06 | 14.9 | 0.64 | 1.65 | 0.00059 | 0.944 | 200807_s_at | HSPD1/Homo sapiens heat |
| | | | | | | | | | | shock 60 kD protein 1 |
| | | | | | | | | | | (chaperonin) |
| Hs.30054 | 0.8657 | 5.80E−09 | 2.20E−06 | 9.82 | 0.59 | 4.11 | 0.0029 | 0.6932 | 204714_s_at | F5/Homo sapiens |
| | | | | | | | | | | coagulation factor V |
| | | | | | | | | | | (proaccelerin) |
| Hs.75432 | 0.8641 | 6.70E−09 | 2.30E−06 | 9.79 | 0.54 | 2.19 | 0.00045 | 0.8803 | 201892_s_at | IMPDH2/Homo sapiens |
| | | | | | | | | | | IMP (inosine |
| | | | | | | | | | | monophosphate) |
| | | | | | | | | | | dehydrogenase 2 |
| Hs.699 | 0.8593 | 1.10E−08 | 3.10E−06 | 8.5 | 0.59 | 1.62 | 0.37 | 0.8131 | 200967_at | PPIB/Homo sapiens |
| | | | | | | | | | | peptidylprolyl isomerase B |
| | | | | | | | | | | (cyclophilin B) |
| Hs.1708 | 0.855 | 1.60E−08 | 3.80E−06 | 11.07 | 0.56 | 1.72 | 0.14 | 0.8053 | 200910_at | CCT3/Homo sapiens |
| | | | | | | | | | | chaperonin containing TCP1 |
| Hs.69469 | 0.8485 | 2.90E−08 | 6.00E−06 | 8.47 | 0.59 | 1.61 | 0.12 | 0.7948 | 202231_at | GA17/Homo sapiens |
| | | | | | | | | | | dendritic cell protein |
| Hs.82280 | 0.848 | 3.00E−08 | 6.00E−06 | 9.05 | 0.58 | 2.1 | 0.089 | 0.8596 | 204319_s_at | RGS10/Homo sapiens |
| | | | | | | | | | | regulator of G-protein |
| | | | | | | | | | | signaling 10 |
| Hs.79217 | 0.8421 | 5.10E−08 | 8.70E−06 | 8.88 | 0.53 | 1.85 | 1.3 | 0.873 | 202148_s_at | PYCR1/Homo sapiens |
| | | | | | | | | | | pyrroline-5-carboxylate |
| | | | | | | | | | | reductase 1 |
| Hs.117950 | 0.8367 | 8.30E−08 | 1.20E−05 | 12.72 | 0.58 | 1.98 | 0.92 | 0.8066 | 201013_s_at | SAICAR/multifunctional |
| | | | | | | | | | | polypeptide similar to |
| | | | | | | | | | | SAICAR synthetase and |
| | | | | | | | | | | AIR carboxylase |
| Hs.8858 | 0.833 | 1.10E−07 | 1.50E−05 | 8.54 | 0.56 | 1.66 | 0.11 | 0.8151 | 217986_s_at | BAZIA/Homo sapiens |
| | | | | | | | | | | bromodomain adjacent to |
| | | | | | | | | | | zinc finger domain. |
| Hs.75939 | 0.8287 | 1.70E−07 | 2.00E−05 | 9.15 | 0.48 | 1.88 | 0.019 | 0.8333 | 209825_s_at | UMPK/Homo sapiens |
| Hs.75061 | 0.8233 | 2.60E−07 | 2.70E−05 | 7.98 | 0.46 | 1.98 | 0.054 | 0.8835 | 200644_at | MACMARCKS/Homo |
| | | | | | | | | | | sapiens macrophage |
| | | | | | | | | | | myristoylated alanine-rich C |
| | | | | | | | | | | kinase substrate |
| Hs.162209 | 0.8217 | 3.00E−07 | 3.10E−05 | 16.32 | 0.56 | 2.89 | 0.014 | 0.7902 | 214598_at | CLDN8/DKFZp564/ |
| | | | | | | | | | | Claudin 8 Homo sapiens |
| | | | | | | | | | | mRNA |
| Hs.154672 | 0.8147 | 5.40E−07 | 4.70E−05 | 8.96 | 0.53 | 1.74 | 0.046 | 0.8314 | 201761_at | MTHFD1/Homo sapiens |
| | | | | | | | | | | methylene tetrahydrofolate |
| | | | | | | | | | | dehydrogenase (NAD+ |
| | | | | | | | | | | dependent) |
| Hs.18910 | 0.8115 | 7.10E−07 | 5.70E−05 | 6.91 | 0.52 | 1.89 | 0.037 | 0.7111 | 204394_at | POV1(SLC43A1)/Homo |
| | | | | | | | | | | sapiens prostate cancer |
| | | | | | | | | | | overexpressed gene 1 |
| Hs.109059 | 0.8104 | 7.70E−07 | 6.10E−05 | 7.4 | 0.52 | 1.84 | 0.027 | 0.8091 | 203931_s_at | MRPL12/Homo sapiens |
| | | | | | | | | | | mitochondrial ribosomal |
| | | | | | | | | | | protein L12 |
| Hs.98732 | 0.8104 | 7.70E−07 | 6.00E−05 | 5.45 | 0.42 | 6.42 | 0.01 | 0.8083 | 215432_at | EIF3S8/Homo sapiens |
| | | | | | | | | | | Chromosome 16 BAC clone |
| | | | | | | | | | | CIT987SK-A-923A4 |

TABLE 45b

| SEQ ID | UniGene | Unigene | |
|---|---|---|---|
| NO | (archival) | (current) | Target Description |
| 1 | Hs.7780 | Hs.480311 | Consensus includes gb: AV715767 /FEA = EST /DB_XREF = gi: 10797284 |
| | | | /DB_XREF = est: AV715767 /CLONE = DCBATH02 /UG = Hs.7780 Homo |
| | | | sapiens mRNA; cDNA DKFZp564A072 (from clone DKFZp564A072) |
| 2 | Hs.21293 | Hs.492859 | gb: S73498.1 /DEF = Homo sapiens AgX-1 antigen mRNA; complete cds. |
| | | | /FEA = mRNA /PROD = AgX-1 antigen /DB_XREF = gi: 688010 |
| | | | /UG = Hs.21293 UDP-N-acteylglucosamine pyrophosphorylase 1 |
| | | | /FL = gb: AB011004.1 gb: NM_003115.1 gb: S73498.1 |
| 3 | Hs.79037 | Hs.632539 | gb: NM_002156.1 /DEF = Homo sapiens heat shock 60 kD protein 1 |
| | | | (chaperonin) (HSPD1); mRNA. /FEA = mRNA /GEN = HSPD1 /PROD = heat |
| | | | shock 60 kD protein 1 (chaperonin) /DB_XREF = gi: 4504520 |
| | | | /UG = Hs.79037 heat shock 60 kD protein 1 (chaperonin) |
| | | | /FL = gb: BC002676.1 gb: BC003030.1 gb: M34664.1 gb: M22382.1 |
| | | | gb: NM_002156.1 |
| 4 | Hs.30054 | Hs.30054 | gb: NM_000130.2 /DEF = Homo sapiens coagulation factor V (proaccelerin; |
| | | | labile factor) (F5); mRNA. /FEA = mRNA /GEN = F5 /PROD = coagulation |
| | | | factor V precursor /DB_XREF = gi: 10518500 /UG = Hs.30054 coagulation |
| | | | factor V (proaccelerin; labile factor) /FL = gb: NM_000130.2 gb: M16967.1 |
| | | | gb: M14335.1 |
| 5 | Hs.75432 | Hs.654400 | gb: NM_000884.1 /DEF = Homo sapiens IMP (inosine monophosphate) |
| | | | dehydrogenase 2 (IMPDH2); mRNA. /FEA = mRNA /GEN = IMPDH2 |
| | | | /PROD = IMP (inosine monophosphate) dehydrogenase 2 |
| | | | /DB_XREF = gi: 4504688 /UG = Hs.75432 IMP (inosine monophosphate) |
| | | | dehydrogenase 2 /FL = gb: J04208.1 gb: NM_000884.1 |
| 6 | Hs.699 | Hs.434937 | gb: NM_000942.1 /DEF = Homo sapiens peptidylprolyl isomerase B |
| | | | (cyclophilin B) (PPIB); mRNA. /FEA = mRNA /GEN = PPIB |
| | | | /PROD = peptidylprolyl isomerase B (cyclophilin B) |
| | | | /DB_XREF = gi: 4758949 /UG = Hs.699 peptidylprolyl isomerase B |
| | | | (cyclophilin B) /FL = gb: BC001125.1 gb: M60857.1 gb: M63573.1 |
| | | | gb: NM_000942.1 |
| 7 | Hs.1708 | Hs.491494 | gb: NM_005998.1 /DEF = Homo sapiens chaperonin containing TCP1; |
| | | | subunit 3 (gamma) (CCT3); mRNA. /FEA = mRNA /GEN = CCT3 |
| | | | /PROD = chaperonin containing TCP1; subunit 3 (gamma) |
| | | | /DB_XREF = gi: 5174726 /UG = Hs.1708 chaperonin containing TCP1; |
| | | | subunit 3 (gamma) /FL = gb: NM_005998.1 |
| 8 | Hs.69469 | Hs.502244 | gb: NM_006360.1 /DEF = Homo sapiens dendritic cell protein (GA17); |
| | | | mRNA. /FEA = mRNA /GEN = GA17 /PROD = dendritic cell protein |
| | | | /DB_XREF = gi: 5453653 /UG = Hs.69469 dendritic cell protein |
| | | | /FL = gb: AF277183.1 gb: AF064603.1 gb: NM_006360.1 |
| 9 | Hs.82280 | Hs.501200 | gb: NM_002925.2 /DEF = Homo sapiens regulator of G-protein signaling 10 |
| | | | (RGS10); mRNA. /FEA = mRNA /GEN = RGS10 /PROD = regulator of G- |
| | | | protein signaling 10 /DB_XREF = gi: 11184225 /UG = Hs.82280 regulator of |
| | | | G-protein signaling 10 /FL = gb: NM_002925.2 gb: AF045229.1 |
| 10 | Hs.79217 | Hs.458332 | gb: NM_006907.1 /DEF = Homo sapiens pyrroline-5-carboxylate reductase 1 |
| | | | (PYCR1); nuclear gene encoding mitochondrial protein; mRNA. |
| | | | /FEA = mRNA /GEN = PYCR1 /PROD = pyrroline-5-carboxylate reductase 1 |
| | | | /DB_XREF = gi: 5902035 /UG = Hs.79217 pyrroline-5-carboxylate reductase |
| | | | 1 /FL = gb: M77836.1 gb: NM_006907.1 |
| 11 | Hs.117950 | Hs.518774 | Consensus includes gb: AA902652 /FEA = EST /DB_XREF = gi: 3037775 |
| | | | /DB_XREF = est: ok71a12.s1 /CLONE = IMAGE: 1519390 /UG = Hs.117950 |
| | | | multifunctional polypeptide similar to SAICAR synthetase and AIR |
| | | | carboxylase /FL = gb: NM_006452.1 |
| 12 | Hs.8858 | Hs.509140 | gb: NM_013448.1 /DEF = Homo sapiens bromodomain adjacent to zinc |
| | | | finger domain; 1A (BAZ1A); mRNA. /FEA = mRNA /GEN = BAZ1A |
| | | | /PROD = bromodomain adjacent to zinc finger domain; 1A |
| | | | /DB_XREF = gi: 7304918 /UG = Hs.8858 bromodomain adjacent to zinc |
| | | | finger domain; 1A /FL = gb: AB032252.1 gb: NM_013448.1 |
| | | | Bromodomain adjacent to zinc finger domain protein 1A (ATP-utilizing |
| | | | chromatin assembly and remodeling factor 1) (hACF1) (ATP-dependent |
| | | | chromatin remodelling protein) (Williams syndrome transcription factor- |
| | | | related chromatin remodeling factor 180) (WCRF180) (hWALp1) |
| | | | (CHRAC subunit ACF1) (HSPC317). From SPD |
| 13 | Hs.75939 | Hs.458360 | gb: BC002906.1 /DEF = Homo sapiens; Similar to uridine monophosphate |
| | | | kinase; clone MGC: 10318; mRNA; complete cds. /FEA = mRNA |
| | | | /PROD = Similar to uridine monophosphate kinase |
| | | | /DB_XREF = gi: 12804106 /UG = Hs.75939 uridine monophosphate kinase |
| | | | /FL = gb: BC002906.1 gb: AF236637.1 |
| 14 | Hs.75061 | Hs.75061 | gb: NM_023009.1 /DEF = Homo sapiens macrophage myristoylated alanine- |
| | | | rich C kinase substrate (MACMARCKS); mRNA. /FEA = mRNA |
| | | | /GEN = MACMARCKS /PROD = macrophage myristoylated alanine-rich C |
| | | | kinasesubstrate /DB_XREF = gi: 13491173 /UG = Hs.75061 macrophage |
| | | | myristoylated alanine-rich C kinase substrate /FL = gb: NM_023009.1 |
| 15 | Hs.162209 | Hs.162209 | Consensus includes gb: AL049977.1 /DEF = Homo sapiens mRNA; cDNA |
| | | | DKFZp564C122 (from clone DKFZp564C122). /FEA = mRNA |
| | | | /DB_XREF = gi: 4884227 /UG = Hs.162209 claudin 8 /FL = gb: NM_012132.1 |
| 16 | Hs.154672 | Hs.469030 | gb: NM_006636.2 /DEF = Homo sapiens methylene tetrahydrofolate |
| | | | dehydrogenase (NAD+ dependent); methenyltetrahydrofolate |
| | | | cyclohydrolase (MTHFD2); nuclear gene encoding mitochondrial protein; |
| | | | mRNA. /FEA = mRNA /GEN = MTHFD2 /PROD = methylene |
| | | | tetrahydrofolate dehydrogenase (NAD+dependent); |
| | | | methenyltetrahydrofolate cyclohydrolase; precursor |
| | | | /DB_XREF = gi: 13699869 /UG = Hs.154672 methylene tetrahydrofolate |
| | | | dehydrogenase (NAD+ dependent); methenyltetrahydrofolate |
| | | | cyclohydrolase /FL = gb: NM_006636.2 |
| 17 | Hs.18910 | Hs.591952 | gb: NM_003627.1 /DEF = Homo sapiens prostate cancer overexpressed gene |
| | | | 1 (POV1); mRNA. /FEA = mRNA /GEN = POV1 /PROD = prostate cancer |
| | | | overexpressed gene 1 /DB_XREF = gi: 4505970 /UG = Hs.18910 prostate |
| | | | cancer overexpressed gene 1 /FL = gb: BC001639.1 gb: AF045584.1 |
| | | | gb: NM_003627.1 |
| 18 | Hs.109059 | Hs.109059 | gb: NM_002949.1 /DEF = Homo sapiens mitochondrial ribosomal protein |
| | | | L12 (MRPL12); mRNA. /FEA = mRNA /GEN = MRPL12 |
| | | | /PROD = mitochondrial ribosomal protein L12 /DB_XREF = gi: 4506672 |
| | | | /UG = Hs.109059 mitochondrial ribosomal protein L12 /FL = gb: BC002344.1 |
| | | | gb: U25041.1 gb: AF105278.1 gb: NM_002949.1 |
| | Hs.98732 | Hs.306812 | Consensus includes gb: AC003034 /DEF = Homo sapiens Chromosome 16 |
| | | | BAC clone CIT987SK-A-923A4 /FEA = mRNA_2 /DB_XREF = gi: 3219338 |
| | | | /UG = Hs.98732 Homo sapiens Chromosome 16 BAC clone CIT987SK-A- |
| | | | 923A4 /FEA = mRNA_2 /DB_XREF = gi: 3219338 /UG = Hs.98732 Homo |
| | | | sapiens Chromosome 16 BAC clone CIT987SK-A-923A4 |

The following information provides further description of the top 3 ranking genes selected by RFE-SVM based upon public databases and references:

### DKFZp564 (Hs.7780 Old Cluster; Hs.480311 New Cluster) (SEQ ID NO. 1)

Homo sapiens mRNA; cDNA DKFZp564A072 (from clone DKFZp564A072)

Chromosome location: Chr.4, 527.0 cR

Summary: LIM domains are cysteine-rich double zinc fingers composed of 50 to 60 amino acids that are involved in protein-protein interactions. LIM domain-containing proteins are scaffolds for the formation of multiprotein complexes. The proteins are involved in cytoskeleton organization, cell lineage specification, organ development, and oncogenesis. Enigma family proteins (see ENIGMA; MIM 605900) possess a 100-amino acid PDZ domain in the N terminus and 1 to 3 LIM domains in the C terminus.[supplied by OMIM].
Genbank entry (with sequence): AL049969.1
Protein Product: PDZ and LIM-domain 5 (PDLIM5) (SEQ ID NO. 19)
cDNA SOURCES: Liver and Spleen, bone, brain, breast-normal, colon, heart, kidney, lung, mixed, ovary, pancreas, placenta, pooled, prostate, skin, stomach, testis, uterus, vascular, whole blood.

### AgX-1/UAP1 (Hs.21293 old Cluster; Hs.492859 New Cluster) (SEQ ID NO. 2)

UDP-N-acteylglucosamine pyrophosphorylase 1
Chromosome location: 1q23.3
Sequence from GenBank for S73498
Enzyme EC number: 2.7.7.23

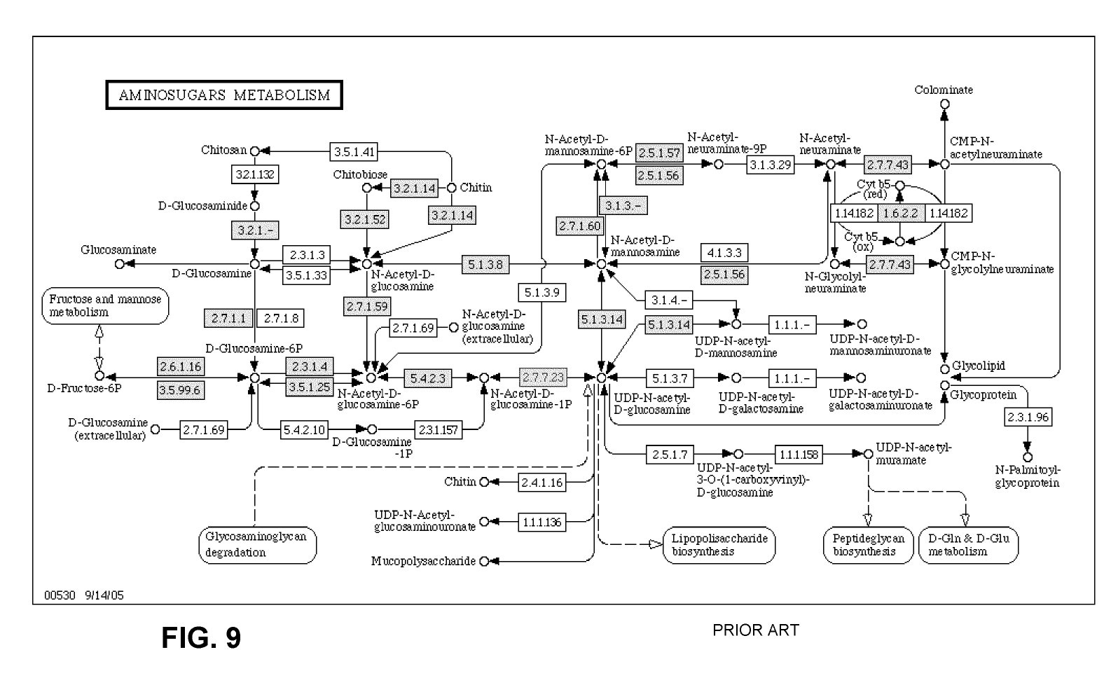

Enzyme involved in aminosugars metabolism (see Kegg pathway around AgX-1/UAP1/SPAG2, FIG. 9).

Reported to be an androgen responsive gene in:

“Transcriptional programs activated by exposure of human prostate cancer cells to androgen”, S. E. DePrimo, et al., Genome Biology 2002, 3:research0032.1-032.12

Reported to be possibly implicated in cancer:

Other interesting alias: SPAG 2: sperm associated antigen 2. Has been connected to male infertility:
“Expression of the human antigen SPAG2 in the testis and localization to the outer dense fibers in spermatozoa”, Diekman, A. B., et al., Mol. Reprod. Dev. 1998 July; 50(3):284-93.
Tissue specificity: Widely expressed. Isoform AGX1 is more abundant in testis than isoform AGX2, while isoform AGX2 is more abundant than isoform AGX1 in somatic tissue. Expressed at low level in placenta, muscle and liver.
Protein product: AgX-1 antigen, accession S73498.1 (SEQ ID NO. 20)
Secreted: May be present in blood and tissue.

UAP 1 is correlated with genes involved in mitochondrial activity, including AMACR, and HSDP1.

### HSPD1 (Hs.79037 old Cluster; Hs.632539 New Cluster) (SEQ ID NO. 3)

### Chaperonin

Chromosome Location: 2q33.1

Function: This gene encodes a member of the chaperonin family. The encoded mitochondrial protein may function as a signaling molecule in the innate immune system. This protein is essential for the folding and assembly of newly imported proteins in the mitochondria. This gene is adjacent to a related family member and the region between the 2 genes functions as a bidirectional promoter.

Protein product: chaperonin heat shock 60 kD protein 1 (chaperonin); heat shock protein 65; mitochondrial matrix protein PI; P60 lymphocyte protein; short heat shock protein 60 Hsp60s 1; Accession NP_{—}002147 (SEQ ID NO. 21)

HSPD1 is correlated with genes involved in mitochondrial activity.

### F5 (Hs.30054) (SEQ ID NO. 4)

F5 coagulation factor V precursor

Chromosome Location: 1 q23

Function: This gene encodes coagulation factor V which is an essential factor of the blood coagulation cascade. This factor circulates in plasma, and is converted to the active form by the release of the activation peptide by thrombin during coagulation. This generates a heavy chain and a light chain which are held together by calcium ions. The active factor V is a cofactor that participates with activated coagulation factor X to activate prothrombin to thrombin.

Protein Product: coagulation factor V precursor [Homo sapiens], Accession NP_{—}000121 (SEQ ID NO. 22)

### IMPDH2 (Hs.75432 Old Cluster; Hs.476231 New Cluster) (SEQ ID NO. 5)

IMP (inosine monophosphate) dehydrogenase 2

Chromosome location: Location: 3p21.2

Unigene cluster: Hs.476231

Enzyme: EC 1.1.1.205

Function: This gene encodes the rate-limiting enzyme in the de novo guanine nucleotide biosynthesis. It is thus involved in maintaining cellular guanine deoxy- and ribonucleotide pools needed for DNA and RNA synthesis. The encoded protein catalyzes the NAD-dependent oxidation of inosine-5′-monophosphate into xanthine-5′-monophosphate, which is then converted into guanosine-5′-monophosphate. This gene is up-regulated in some neoplasms, suggesting it may play a role in malignant transformation.

Protein product: inosine monophosphate dehydrogenase 2 [Homo sapiens];

Accession NP_{—}000875 (SEQ ID NO. 23)

Sequences: Source sequence J04208:

Related to apoptosis.

### PPIB (Hs.699 Old Cluster; Hs.434937 New Cluster) (SEQ ID NO. 6)

Peptidylprolyl isomerase B precursor (cyclophilin B)

Chromosome location: 15q21-q22

Function: The protein encoded by this gene is a cyclosporine-binding protein and is mainly located within the endoplasmic reticulum. It is associated with the secretory pathway and released in biological fluids. This protein can bind to cells derived from T- and B-lymphocytes, and may regulate cyclosporine A-mediated immunosuppression.

Protein Product: peptidylprolyl isomerase B precursor; Accession NP_{—}000933 (SEQ ID NO. 24)

EC_number: 5.2.1.8

Cyclophilin B; peptidyl-prolyl cis-trans isomerase B; PPlase; cyclophilin-like protein; S-cyclophilin; rotamase

Related to apoptosis.

### CTT3 (Hs.1708 old Cluster; Hs:491-494 New Cluster) (SEQ ID NO. 7)

Chaperonin containing TCP 1, subunit 3 (gamma)

Chromosome Location: 1q23

Function: This gene encodes a molecular chaperone that is member of the chaperonin containing TCP1 complex (CCT), also known as the TCP1 ring complex (TRiC). This complex consists of two identical stacked rings, each containing eight different proteins. Unfolded polypeptides enter the central cavity of the complex and are folded in an ATP-dependent manner. The complex folds various proteins, including actin and tubulin.

Protein product: chaperonin containing TCP1, subunit 3 isoform a; Accession NP_{—}005989 (SEQ ID NO. 25)

TCP 1 (t-complex-1) ring complex, polypeptide 5; T-complex protein 1, gamma subunit.

### GA17 (Hs.69469 Old Cluster; Hs.502244 New Cluster) (SEQ ID NO. 8)

Dendritic cell protein (GA17)

Chromosome location: 11p13

Function: HFLB5 encodes a broadly expressed protein containing putative membrane fusion domains that act as a receptor or coreceptor for entry of herpes simplex virus (HSV).

Protein product: Eukaryotic translation initiation factor 3, subunit M ACCESSION NP_{—}006351. (SEQ ID NO. 26)

### RGS10 (Hs.82280 Old Cluster; Hs.501200 New Cluster) (SEQ ID NO. 9)

regulator of G-protein signaling 10 isoform b

Chromosome Location: 10q25

Function: Regulator of G protein signaling (RGS) family members are regulatory molecules that act as GTPase activating proteins (GAPs) for G alpha subunits of heterotrimeric G proteins. RGS proteins are able to deactivate G protein subunits of the Gi alpha, Go alpha and Gq alpha subtypes. They drive G proteins into their inactive GDP-bound forms. Regulator of G protein signaling 10 belongs to this family. All RGS proteins share a conserved 120-amino acid sequence termed the RGS domain. This protein associates specifically with the activated forms of the two related G-protein subunits, G-alphai3 and G-alphaz but fails to interact with the structurally and functionally distinct G-alpha subunits. Regulator of G protein signaling 10 protein is localized in the nucleus.

Protein product: regulator of G-protein signaling 10 isoform b; Accession NP_{—}002916 (SEQ ID NO. 27)

Related to apoptosis.

### PYCR1 (Hs.79217 Old Cluster; Hs.458332 New Cluster) (SEQ ID NO. 10)

Pyrroline-5-carboxylate reductase 1 isoform 1

Chromosome location: 17q25.3

Function: This gene encodes an enzyme that catalyzes the NAD(P)H-dependent conversion of pyrroline-5-carboxylate to proline. This enzyme may also play a physiologic role in the generation of NADP(+) in some cell types. The protein forms a homopolymer and localizes to the mitochondrion.

Protein product: pyrroline-5-carboxylate reductase 1 isoform 1; Accession NP_{—}008838 (SEQ ID NO. 28)

Related to mitochrondrial function.

Additional information about the remaining top 19 genes and their protein products are found in accompanying sequence listings and on the NCBI database.

Using a subset of 100 genes that were significantly overexpressed in cancer in both the Stamey 2003 data and public data, a number of relevant pathways were identified using a pathway database compiled by MIT. (See, e.g., Subramanian, A., et al., “Gene set enrichment analysis: A knowledge-based approach for interpreting genome-wide expression profiles” (2005) Proc. Natl. Acad. Sci. USA 102, 15545-15550.) This pathway database contains lists of genes from various sources and is highly redundant. The pathways were grouped using a clustering method according to their overlap in number of genes found overexpressed in cancer, then manually verified that the groups were meaningful, to produce a simplified and more robust pathway analysis. Additional information was obtained from the Secreted Protein Database (SPD). (Chen, Y. et al., “SPD—a web-based secreted protein database”, (2005) Nucleic Acids Res. 33 Database Issue: D169-173.)

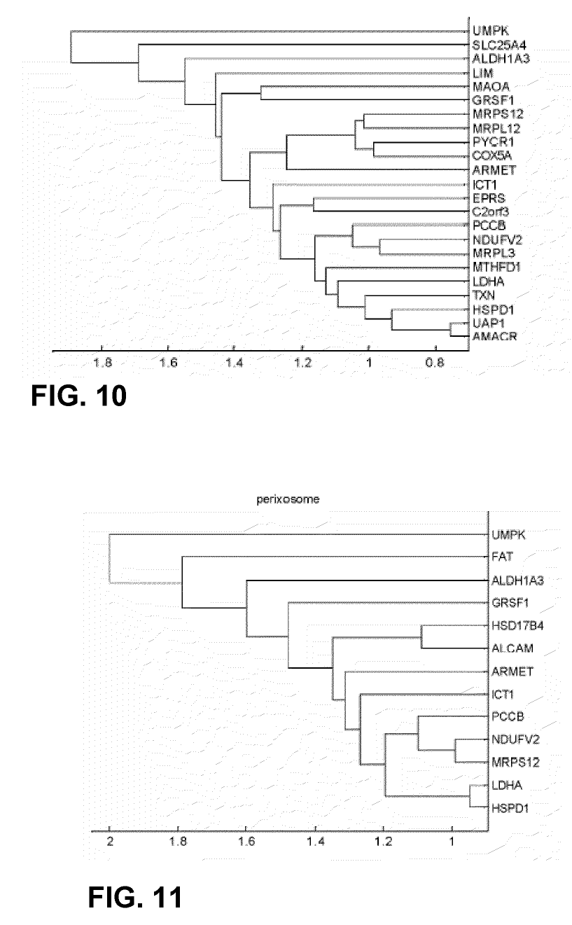

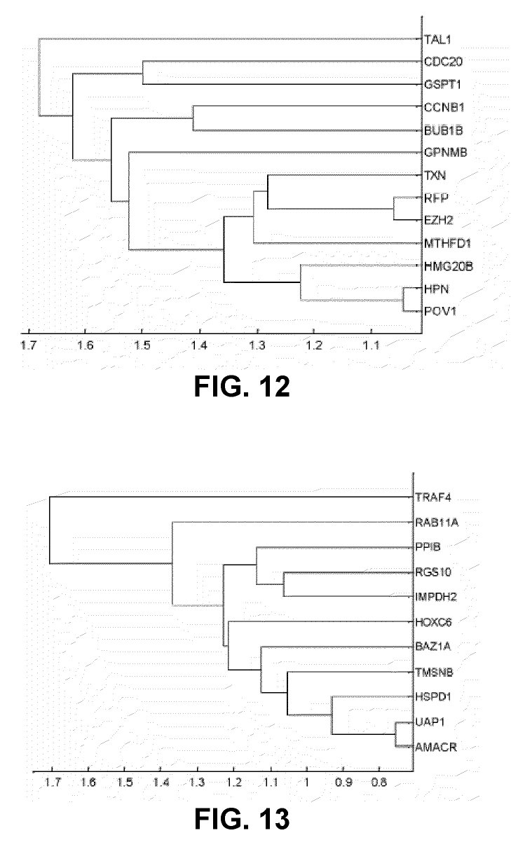

Four main clusters were identified: (1) mitochrondrial genes (UMPK, SLC25A4, ALDH1A3, LIM, MAOA, GRSF1, MRPS12, MRPL12, PYCR1, COX5A, ARMET, ICT1, EPRS, C2orf3, PCCB, NDUFV2, MRPL3, MTHFD1, LDHA, TXN, HSPD1, UAP 1, AMACR) (clustering of these genes is shown in FIG. 10); (2) genes related to perixosome and cell adhesion (UMPK, FAT, ALDH1A3, GRSF1, HSD17B4, ALCAM, ARMET, ICT1, PCCB, NDUFV2, MRPS12, LDHA, HSPD1)(clustering of these genes is shown in FIG. 11); (3) cell proliferation and growth (TAL1, CDC20, GSPT1, CCNB1, BUB1B, GPNMB, TXN, RFP, EXH2, MTHFD1, HMG20B, HPN, POV1)(clustering of these genes is shown in FIG. 12); and (4) genes related to apoptosis, or the p53/p73 signaling pathways (TRAF4, RAB11A, PPIB, RGS10, IMPDH2, HOXC6, BAZ1A, TMSNB, HSPD1, UAP1, AMACR) (clustering of these genes is shown in FIG. 13). Additional pathways that were less represented, but which may be linked to cancer include coagulation and angiogenesis; cell structure/cyotskeleton/actin; DNA damage/repair; HOX-related genes; and kinases.

Because many of the genes identified in the study involved mitochondrial activity and/or apoptosis, it is hypothesized that mitochrondrial apoptosis plays a role in prostate cancer.

P53 has both transcriptional activity that mediates cell cycle arrest and induces mitochondrial apoptosis, possibly via interactions with the Bcl-2 protein family and rendering the membrane of the mitochondrion permeable. Because of the known role of p53 mutations in many cancers, the expression levels of p53 and related genes like p73 were investigated and found to be strongly underexpressed in the cancer tissue in the datasets that were used in the study. Connections are apparent between mitochondrial activity and apoptosis.

The preceding detailed description of the preferred embodiments disclosed methods for identification of biomarkers for prostate cancer using gene expression data from microarrays. RFE-SVM was used to identify a small number of biomarkers that should lead to the creation of inexpensive, accurate tests that may be used in conjunction with or in place of current diagnostic, prognostic and monitoring tests for prostate cancer by using gene expression or protein expression data. Preferred applications of the present invention will target proteins expressed by the identified genes that are detectable in serum or semen, thus providing non-invasive or minimally invasive screening for prostate cancer and monitoring of treatment.

Alternative embodiments of the present invention will become apparent to those having ordinary skill in the art to which the present invention pertains. Such alternate embodiments are considered to be encompassed within the spirit and scope of the present invention. Accordingly, the scope of the present invention is described by the appended claims and is supported by the foregoing description.

### REFERENCES

### Incorporated Herein by Reference

[1] Singh D, et al., Gene expression correlates of clinical prostate cancer behavior Cancer Cell, 2:203-9, Mar. 1, 2002.
[2] Febbo P., et al., Use of expression analysis to predict outcome after radical prostatectomy, The Journal of Urology, Vol. 170, pp. S11-S20, December 2003. Delineation of prognostic biomarkers in prostate cancer. Dhanasekaran S M, Barrette T R, Ghosh D, Shah R, Varambally S, Kurachi K, Pienta K J, Rubin M A, Chinnaiyan A M. Nature. 2001 Aug. 23; 412(6849):822-6.
[3] Comprehensive gene expression analysis of prostate cancer reveals distinct transcriptional programs associated with metastatic disease. LaTulippe E, Satagopan J, Smith A, Scher H, Scardino P, Reuter V, Gerald W L. Cancer Res. 2002 Aug. 1; 62(15):4499-506.
[4] Gene expression analysis of prostate cancers. Luo J H, Yu Y P, Cieply K, Lin F, Deflavia P, Dhir R, Finkelstein S, Michalopoulos G, Becich M. Mol Carcinog. 2002 January; 33(1):25-35
[5] Expression profiling reveals hepsin overexpression in prostate cancer. Magee J A, Araki T, Patil S, Ehrig T, True L, Humphrey P A, Catalona W J, Watson M A, Milbrandt J. Cancer Res. 2001 Aug. 1; 61(15):5692-6.
[6] Analysis of gene expression identifies candidate markers and pharmacological targets in prostate cancer. Welsh J B, Sapinoso L M, Su Al, Kern S G, Wang-Rodriguez J, Moskaluk C A, Frierson H F Jr, Hampton G M. Cancer Res. 2001 Aug. 15; 61(16):5974-8.
[7] Human prostate cancer and benign prostatic hyperplasia: molecular dissection by gene expression profiling. Luo J, Duggan D J, Chen Y, Sauvageot J, Ewing C M, Bittner M L, Trent J M, Isaacs W B. Cancer Res. 2001 Jun. 15; 61(12):4683-8.
[8] A molecular signature of metastasis in primary solid tumors. Ramaswamy S, Ross K N, Lander E S, Golub T R. Nat Genet. 2003 January; v33(1):49-54. Epub 2002 Dec. 9.
[9] A compendium of gene expression in normal human tissues. Hsiao L L, Dangond F, Yoshida T, Hong R, Jensen R V, Misra J, Dillon W, Lee K F, Clark K E, Haverty P, Weng Z, Mutter G L, Frosch M P, Macdonald M E, Milford E L, Crum C P, Bueno R, Pratt R E, Mahadevappa M, Warrington J A, Stephanopoulos G, Stephanopoulos G, Gullans S R. Physiol Genomics. 2001 Dec. 21; 7(2):97-104.
[10] Molecular classification of human carcinomas by use of gene expression signatures. Su A I, Welsh J B, Sapinoso L M, Kern S G, Dimitrov P, Lapp H, Schultz P G, Powell S M, Moskaluk C A, Frierson H F Jr, Hampton G M. Cancer Res. 2001 Oct. 15; 61(20):7388-93.
[11] Gene expression analysis of prostate cancers. Jian-Hua Luo*, Yan Ping Yu, Kathleen Cieply, Fan Lin, Petrina Deflavia, Rajiv Dhir, Sydney Finkelstein, George Michalopoulos, Michael Becich.
[12] Transcriptional Programs Activated by Exposure of Human Prostate Cancer Cells to Androgen”, Samuel E. DePrimo, Maximilian Diehn, Joel B. Nelson, Robert E. Reiter, John Matese, Mike Fero, Robert Tibshirani, Patrick O. Brown, James D. Brooks. Genome Biology, 3(7) 2002
[13] A statistical method for identifying differential gene-gene co-expression patterns, Yinglei Lai, Baolin Wu, Liang Chen and Hongyu Zhao. Bioinformatics vol. 20 issue 17.
[14] Induction of the Cdk inhibitor p21 by LY83583 inhibits tumor cell proliferation in a p53-independent manner
Dimitri Lodygin, Antje Menssen, and Heiko Hermeking, J. Clin. Invest. 110: 1717-1727 (2002).
[15] Classification between normal and tumor tissues based on the pair-wise gene expression ratio. YeeLeng Yap, XueWu Zhang, M T Ling, XiangHong Wang, Y C Wong, and Antoine Danchin B M C Cancer. 2004; 4: 72.
[16] Kishino H, Waddell P J. Correspondence analysis of genes and tissue types and finding genetic links from microarray data. Genome Inform Ser Workshop Genome Inform 2000; 11: 83-95.
[17] Proteomic analysis of cancer-cell mitochondria. Mukesh Verma, Jacob Kagan, David Sidransky & Sudhir Srivastava, Nature Reviews Cancer 3, 789-795 (2003);
[18] Changes in collagen metabolism in prostate cancer: a host response that may alter progression. Burns-Cox N, Avery N C, Gingell J C, Bailey A J. J. Urol. 2001 November; 166(5):1698-701.
[19] Differentiation of Human Prostate Cancer PC-3 Cells Induced by Inhibitors of Inosine 5′-Monophosphate Dehydrogenase. Daniel Florykl, Sandra L. Tollaksen2, Carol S. Giometti2 and Eliezer Hubermanl Cancer Research 64, 9049-9056, Dec. 15, 2004.
[20] Epithelial Na, K-ATPase expression is down-regulated in canine prostate cancer; a possible consequence of metabolic transformation in the process of prostate malignancy Ali Mobasheri, Richard Fox, lain Evans, Fay Cullingham, Pablo Martin-Vasallo and Christopher S Foster Cancer Cell International 2003, 3:8

1. A method for screening, predicting or monitoring prostate cancer comprising:
detecting within a patient sample from one to eight proteins selected from the group consisting of PDZ and LIM5 (SEQ ID NO. 19), UAP1/AgX1 antigen (SEQ ID NO. 20), HSPD1/chaperonin (SEQ ID NO. 21), IMPDH2 (SEQ ID NO. 22), F5 (SEQ ID NO. 23), PPIB (SEQ ID NO. 24), RGS10 (SEQ ID NO. 25) and PYCR1 (SEQ ID NO. 28); and
comparing detected levels of the from one to eight proteins to the same proteins in a normal prostate sample;
wherein increased of the proteins in the patient sample is indicative of prostate cancer.

2. The method of claim 1, wherein the patient sample comprises serum and the protein comprises PDZ and LIM5 (SEQ ID NO. 19).

3. The method of claim 1, wherein the proteins are expressed within an apoptosis pathway and the proteins comprise UAP1/AgX1 antigen (SEQ ID NO. 20), IMPDH2 (SEQ ID NO. 22), PPIB (SEQ ID NO. 24) and RGS10 (SEQ ID NO. 25).

4. The method of claim 1, wherein the proteins are expressed within mitochondrial activity pathway and the proteins comprise UAP1/AgX1 antigen (SEQ ID NO. 20), HSPD1/chaperonin (SEQ ID NO. 21) and PYCR1 (SEQ ID NO. 28).

5. The method of claim 1, wherein the patient sample comprises semen and the protein comprises UAP1/AgX1 antigen (SEQ ID NO. 20).

6. A method for screening, predicting or monitoring prostate cancer comprising:
detecting within a patient sample overexpression relative to normal of any combination of four proteins selected from the group consisting of PDZ and LIM5 (SEQ ID NO. 19), UAP1/AgX1 antigen (SEQ ID NO. 20), HSPD1/chaperonin (SEQ ID NO. 21), IMPDH2 (SEQ ID NO. 22), F5 (SEQ ID NO. 23), PPIB (SEQ ID NO. 24), RGS10 (SEQ ID NO. 25) and PYCR1 (SEQ ID NO. 28).

7. The method of claim 6, wherein the four proteins comprise PDZ and LIM5 (SEQ ID NO. 19), UAP1/AgX1 antigen (SEQ ID NO. 20), HSPD1/chaperonin (SEQ ID NO. 21) and IMPDH2 (SEQ ID NO. 22).

8. The method of claim 6, wherein the four proteins comprise PDZ and LIM5 (SEQ ID NO. 19), UAP1/AgX1 antigen (SEQ ID NO. 20), HSPD1/chaperonin (SEQ ID NO. 21) and F5 (SEQ ID NO. 23).

9. A method for screening, predicting or monitoring prostate cancer comprising:
detecting within a patient sample overexpression relative to normal of the combination of three proteins consisting of PDZ and LIM5 (SEQ ID NO. 19), UAP1/AgX1 antigen (SEQ ID NO. 20), HSPD1/chaperonin (SEQ ID NO. 21).
